(19) United States
(12) Patent Application Publication
(10) Pub. No.: US 2010/0167925 A1
(43) Pub. Date: Jul. 1, 2010

(54) Substituted Azolin-2-yl-Amino Compounds

(75) Inventors: Christopher Koradin, Ludwigshafen (DE); Markus Kordes, Bobenheim-Roxheim (DE); Ronan Le Vezouet, Mannheim (DE); Deborah L. Culbertson, Fuquay Varina, NC (US); Douglas D. Anspaugh, Apex, NC (US)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/376,455

(22) PCT Filed: Aug. 6, 2007
(86) PCT No.: PCT/EP2007/058109

§ 371 Date: Feb. 5, 2009

Related U.S. Application Data
(60) Provisional application No. 60/836,064, filed on Aug. 7, 2006.

(51) Int. Cl. A01C 1/06; A01N 43/78; C07D 417/02

(52) U.S. Cl. 504/100; 514/370; 548/190

(57) ABSTRACT

The present invention relates to azolin-2-yl-amino compounds of formulae (I. a) and (1.b) and their salts which are useful for combating animal pest, in particular arthropod and nematodes. The present invention also relates to a method for combating such pests and for protecting crops against infestation or infection by such pests. Furthermore, the present invention relates to veterinary compositions for combating animal pests. Formula (I.a), formula (I.b) wherein n is 0 to 4; X is S, O or NR⁵; A is —C(R^{6a})(R^{6b})—, O, NR⁷, S, S(O) or S(O)₂; B is a bond Or CH₂; R¹ is H, CN, C₁-C₆-alkyl, C₂-C₆-alkenyl, C₁-C₆-alkylcarbonyl, C₃-C₆-cycloalkyl, phenyl, benzyl, etc.; R^{2a}, R^{2b} are H, CN, C₁-C₆-alkyl, C₂-C₆-alkenyl, C₁-C₆-alkylcarbonyl, (C₁-C₆-alkyl)-thiocarbonyl, C(O)NR^{a}R^{b}, C(S)NR^{a}R^{b}, (SO₂)NR^{a}R^{b}, phenyl, benzyl, 5 or 6 membered heterocyclic ring etc.; or R¹ together with R^{2a} is C₃-C₅-alkandiyl; or R¹ together with R² are C(O); R^{3a}, R^{3b}, R^{3c}, R^{3d} are H, halogen, CN, C₁-C₆-alkyl, C₁-C₆-alkylamino, C₃-C₆-cycloalkyl, phenyl, benzyl, etc.; R^{4a}, R^{4b} are H, halogen, C₁-C₆-alkyl, C₂-C₆-alkenyl, phenyl, benzyl, 5 or 6 membered hetaryl; or R^{4a} together with R^{b} may also be ═O, ═NR^{C} or ═CR^{d}R^{e}; or R^{2a} together with R^{4a} may form a bridging bivalent radical.

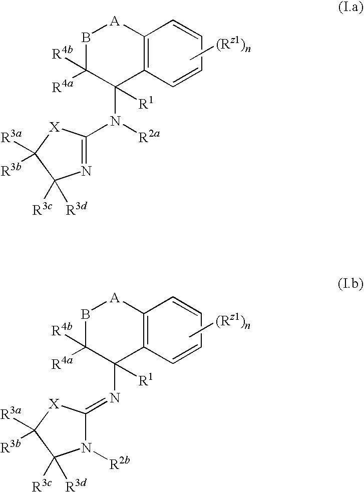

The present invention relates to azolin-2-yl-amino compounds and their salts which are useful for combating animal pest, in particular arthropod and nematodes. The present invention also relates to a method for combating such pests and for protecting crops against infestation or infection by such pests. Furthermore, the present invention relates to veterinary compositions for combating animal pests.

Animal pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating insects, arachnids and nematodes. It is therefore an object of the present invention to provide compounds having a good pesticidal activity and show a broad activity spectrum against a large number of different animal pests, especially against difficult to control insects, arachnids and nematodes.

2-(Indanylamino)-oxazoline compounds and 2-(1,2,3,4-tetrahydronaphtylamino)-oxazoline compounds are described in U.S. Pat. No. 2,870,159, U.S. Pat. No. 2,870,161, U.S. Pat. No. 2,883,410 and U.S. Pat. No. 3,679,798 as regulators of the central nervous system.

2-(Amino)-oxazoline compounds having a fused heterocyclic substituent attached to the amino function are known from U.S. Pat. No. 3,509,170. Those compounds are reported to have a central nervous system depressant effect.

U.S. Pat. No. 3,636,219 discloses 2-(indanylamino)- and 2-(1,2,3,4-tetrahydronaphtylamino)-thiazoline and imidazoline compounds and their use in anticholinergic compositions.

None of the aforementioned documents describes pesticidal activity of 2-amino-azoline compounds.

DE 1963192 discloses inter alia 2-(indan-5-ylamino)oxazoline. The compound is suggested to have an ovicidal activity against certain acarid ectoparasites of animals such as ticks. An insecticidal activity is not mentioned.

The 60/739,730 discloses indanyl- and tetrahydronaphthyl-amino-azoline compounds as well as agricultural compositions comprising them, which are useful for combating animal pests.

It is therefore an object of the present invention to provide compounds having a good pesticidal activity, in particular insecticidal activity, and show a broad activity spectrum against a large number of different animal pests, especially against difficult to control insects.

It has been found that these objectives can be achieved by compounds of formulae (I.a) or (I.b) or a salt thereof,

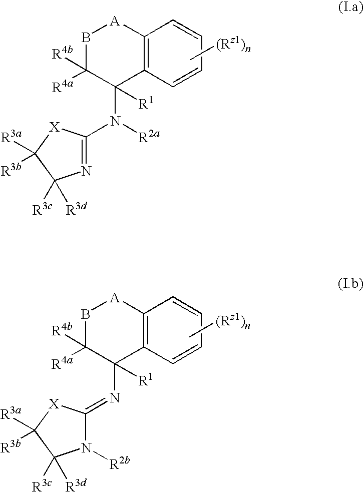

wherein
n is 0, 1, 2, 3 or 4.
X is sulfur, oxygen or a radical NR⁵;
A is —C(R^{6a})(R^{6b})—, oxygen, NR⁷, sulfur, S(O) or S(O)₂;
B is a chemical bond or CH₂;
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
   C₃-C₆-cycloalkyl, phenyl or benzyl, phenoxycarbonyl, 5 or 6 membered hetaryl and 5 or 6 membered hetarylmethyl each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2}, C(O)NR^{a}R^{b}, C(S)NR^{a}R^{b}, (SO₂)NR^{a}R^{b},
   phenyl, benzyl, phenoxycarbonyl, 5 or 6 membered hetarylmethyl, 5 or 6 membered hetarylcarbonyl, a 5 or 6 membered heterocyclic ring and benzoyl each of the seven last mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from oxygen, sulfur and nitrogen as ring members, and wherein the 5 or 6 membered heterocyclic ring contains 1, 2 or 3 heteroatoms selected from oxygen, sulfur and nitrogen as ring members; or
R¹ together with R^{2a} may be C₃-C₅-alkandiyl which may carry 1, 2, 3, 4 or 5 radicals R²¹ and which may be interrupted with 1 or 2 heteroatoms selected from oxygen, sulfur or nitrogen;
R¹ together with R^{2b} may also be a bridging carbonyl group C(O);
R^{3a}, R^{3b}, R^{3c}, R^{3d} are each independently selected from the group consisting of hydrogen, halogen, cyano, nitro, hydroxy, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
   C₃-C₆-cycloalkyl, phenyl or benzyl, each of the last three mentioned radicals may be unsubstituted or may carry any combination of 1, 2, 3, 4 or 5 radicals R^{b3};
R^{4a}, R^{4b} are independently from each other selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
   phenyl, benzyl, 5 or 6 membered hetaryl and 5 or 6 membered hetarylmethyl, each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from oxygen, sulfur and nitrogen as ring members; or
R^{4a} together with R^{4b} may also be ═O, ═NR^{c} or ═CR^{d}R^{e};
R^{2a} together with R^{4a} may form a bridging bivalent radical selected from the group consisting of C(O)—C(R^{24a})(R^{24b}), C(S)—C(R^{24a})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, S(O)—NH.

R⁵ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from oxygen, sulfur and nitrogen as ring members;

R^{6a}, R^{6b} if present are independently of each other selected from the group consisting of hydrogen, halogen, cyano, nitro, hydroxy, mercapto, amino, C₁-C₆-alkyl, C₁-C₆-haloalkyl, C₁-C₆-alkoxy, C₁-C₆-alkylamino, di-(C₁-C₆-alkyl)amino, wherein the carbon atoms in the last 5 mentioned radicals may be unsubstituted or may carry any combination of 1, 2 or 3 radicals R^{a6},
C₃-C₆-cycloalkyl, phenyl or benzyl, each of the last three mentioned radicals may be unsubstituted or may carry any combination of 1, 2, 3, 4 or 5 radicals R^{b6}; or
R^{6a} together with R^{6b} may also be ═O, ═NR^{c} or ═CR^{d}R^{e};
R⁷ if present is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a7},
   C(O)NR^{a}R^{b}, C(S)NR^{a}R^{b}, (SO₂)NR^{a}R^{b},
   phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b7}, and wherein the 5 or 6 membered heteroaromatic ring in hetarylmethyl and hetarylcarbonyl contains 1, 2, 3 or 4 heteroatoms selected from oxygen, sulfur and nitrogen as ring members;
R^{z1} are independently of each other selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-al kenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{az},
   C(O)NR^{a}R^{b}, (SO₂)NR^{a}R^{b}, and radicals of the formula Y-Cy, wherein
   Y is a single bond, oxygen, sulfur or C₁-C₆-alkandiyl, wherein one carbon might be replaced with oxygen,
   Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from oxygen, sulfur and nitrogen as ring members, wherein Cy is unsubstituted or may carry any combination of 1, 2, 3, 4 or 5 radicals R^{bz};
   and wherein two radicals R^{z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, or C₂-C₆-alkynyl, wherein the carbon atoms in these groups may carry any combination of 1, 2 or 3 radicals R^{aw};
R^{c} have one of the meanings given for R^{a} and R^{b} or are independently of each other selected from the group selected from C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from C₁-C₆-alkoxy or di(C₁-C₆-alkyl)amino.
R^{a2}, R^{a3}, R^{a4}, R^{a5}, R^{a6}, R^{a7}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{b6}, R^{b7}, R^{bc} and R^{bz} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a} and R^{24b} have independently one of the meanings given for R^{b1} or two radicals R²¹ bound to the same carbon atom may together with this carbon atom form a carbonyl group;
   provided that if A is —C(R^{6a})(R^{6b}) at least one of the radicals R¹, R^{3a}, R^{3b}, R^{3c}, R^{3d}, R^{4a}, R^{4b}, R^{6a} or R^{6b} is different from hydrogen.

In the compounds of formulae (I.a) and (I.b) the carbon atom which carries R¹ creates a center of chirality. If R^{4a} and R^{4b} and also R^{6a} and R^{6b} are identical, e.g. if R^{4a}, R^{4b}, R^{6a} and R^{6b} are hydrogen, the compounds of the formula (I.a) and (I.b) may exist as enantiomers. If R^{4a} and R^{4b} and/or R^{6a} and R^{6b} are not identical, for example if one, two or three of the last mentioned radicals are different from hydrogen, while the remaining radicals are hydrogen, then the compound may exist as different diastereomers. For example, if R^{4a} is different from hydrogen and R^{4b} is hydrogen, than the aminoazoline moiety may be located cis- or trans with regard to the radical R^{4a}. The present invention relates to every possible stereoisomer of the compounds of general formulae (I.a) and (I.b) as well as to mixtures thereof.

Furthermore, the present invention relates to the use of the compounds of formulae (I.a) and (I.b) for combating arthropod pests or nematodes, in particular for combating insects.

Accordingly, the present invention also provides a method for combating arthropod pests or nematode pests by treating the pests with at least one azoline compound of the general formulae (I.a) or (I.b) and/or a salt thereof.

Furthermore, the present invention provides a method for the control of arthropod pests or nematodes, which comprises contacting the arthropod or nematode pests or their food supply, habitat, breeding ground or their locus with at least one compound of the formulae (I.a) or (I.b) and/or a salt thereof.

Another object of the present invention is a method of protecting growing plants from attack or infestation by arthropod pests or nematodes, which comprises applying to the plants, or to the soil or water in which they are growing, at least one compound of formulae (I.a) or (I.b) and/or an agriculturally acceptable salt thereof.

In a preferred embodiment of the above mentioned methods at least one compound of formulae (I.a) or (I.b) and/or the salt thereof or a composition comprising them is applied in an amount of from 5 g/ha to 2000 g/ha, calculated as the compound of formulae (I.a) or (I.b).

Furthermore, the present invention relates to a method of protection of seed comprising contacting the seeds with at least one compound of formulae (I.a) or (I.b) and/or an agriculturally acceptable salt thereof or a composition containing at least one of these compounds in pesticidally effective amounts. Preferably, at least one compound of formulae (I.a) or (I.b) and/or an agriculturally acceptable salt thereof or a composition comprising at least one of these compounds is applied in an amount of from 0.1 g to 10 kg per 100 kg of seeds.

Accordingly, a further object of the present invention is seed, comprising at least one compound of formulae (I.a) or (I.b) and/or an agriculturally acceptable salt thereof in an amount of from 0.1 g to 10 kg per 100 kg of seeds, calculated as the compound of formulae (I.a) or (I.b).

The present invention also relates to a method for treating, controlling, preventing or protecting animals against infestation or infection by parasites which comprises administering or applying to the animals a parasiticidally effective amount of at least one compound of formulae (I.a) or (I.b) and/or an veterinarily acceptable salt thereof.

Furthermore, the present invention relates to azoline compounds of the general formula (I.a) or (I.b), wherein n, X, A, B, R¹, R^{2a} or R^{2b}, R^{3a}, R^{3b}, R^{3c}, R^{3d}, R^{4a}, R^{4b} and R^{z1} have the meanings given within the description, provided that,
if X is oxygen, then at least one of the radicals R¹ or R^{2a} or R^{2b} is different from hydrogen, and/or A is different from C(R^{6a})(R^{6b})—, oxygen, sulfur, N(CH₃), or if X is oxygen and B is a chemical bond, then A is different from —C(R^{6a})(R^{6b})—,
and provided that, if X is nitrogen or sulfur, then at least one of the radicals R¹ or R^{2a} or R^{2b} is different from hydrogen, and/or A is different from —C(R^{6a})(R^{6b})—,
   and the salts thereof.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae (I.a) or (I.b) with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

Examples of other meanings are:

The term “C₁-C₆-alkyl” as used herein and in the alkyl moieties of C₁-C₆-alkoxy, C₁-C₆-alkylamino, di(C₁-C₆-alkyl)amino, C₁-C₆-alkylthio, C₁-C₆-alkylsulfonyl, C₁-C₆-alkylsulfoxyl, C₁-C₆-alkylcarbonyl, C₁-C₆-alkoxycarbonyl, C₁-C₆-alkylthiocarbonyl, and C₁-C₆-alkylcarbonyloxy refer to a saturated straight-chain or branched hydrocarbon group having 1 to 6 carbon atoms, especially 1 to 4 carbon groups, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. C₁-C₄-alkyl means for example methyl, ethyl, propyl, 1-methylethyl, butyl, 1-methylpropyl, 2-methylpropyl or 1,1-dimethylethyl.

The term “C₁-C₆-haloalkyl” as used herein refers to a straight-chain or branched saturated alkyl group having 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “C₁-C₆-alkoxy” as used herein refers to a straight-chain or branched saturated alkyl group having 1 to 6 carbon atoms (as mentioned above) which is attached via an oxygen atom. Examples include C₁-C₆-alkoxy such as methoxy, ethoxy, OCH₂—C₂H₅, OCH(CH₃)₂, n-butoxy, OCH(CH₃)C₂H₅, OCH₂CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

The term “C₁-C₆-haloalkoxy” as used herein refers to a C₁-C₆-alkoxy group as mentioned above wherein the hydrogen atoms are partially or fully substituted by fluorine, chlorine, bromine and/or iodine, i.e., for example, C₁-C₆-haloalkoxy such as chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-d ichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, nonafluorobutoxy, 5-fluoro-1-pentoxy, 5-chloro-1-pentoxy, 5-bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6-trichloro-1-hexoxy or dodecafluorohexoxy, in particular chloromethoxy, fluoromethoxy, difluoromethoxy, trifluoromethoxy, 2-fluoroethoxy, 2-chloroethoxy or 2,2,2-trifluoroethoxy.

The term “C₁-C₆-alkoxy-C₁-C₆-alkyl” as used herein refers to C₁-C₆-alkyl wherein 1 carbon atom carries a C₁-C₆-alkoxy radical as mentioned above. Examples are CH₂OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy) propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy) butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl, 4-(1,1-dimethylethoxy)butyl and the like.

The term “(C₁-C₆-alkyl)carbonyl” as used herein refers to a straight-chain or branched saturated alkyl group having 1 to 6 carbon atoms (as mentioned above) bonded via the carbon atom of the carbonyl group at any bond in the alkyl group. Examples include C₁-C₆-alkylcarbonyl such C(O)CH₃, C(O)C₂H₅, n-propylcarbonyl, 1-methylethylcarbonyl, n-butylcarbonyl, 1-methylpropylcarbonyl, 2-methylpropylcarbonyl, 1,1-dimethylethylcarbonyl, n-pentylcarbonyl, 1-methylbutylcarbonyl, 2-methylbutylcarbonyl, 3-methylbutylcarbonyl, 1,1-dimethylpropylcarbonyl, 1,2-dimethylpropylcarbonyl, 2,2-dimethylpropylcarbonyl, 1-ethylpropylcarbonyl, n-hexylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl and the like.

The term “(C₁-C₆-alkoxy)carbonyl” as used herein refers to a straight-chain or branched alkoxy group (as mentioned above) having 1 to 6 carbon atoms attached via the carbon atom of the carbonyl group, for example C(O)OCH₃, C(O)OC₂H₅, C(O)O—CH₂—C₂H₅, C(O)OCH(CH₃)₂, n-butoxycarbonyl, C(O)OCH(CH₃)—C₂H₅, C(O)OCH₂CH(CH₃)₂, C(O)OC(CH₃)₃, n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl.

The term “(C₁-C₆-alkyl)carbonyloxy” as used herein refers to a straight-chain or branched saturated alkyl group having 1 to 6 carbon atoms (as mentioned above) bonded via the carbon atom of the carbonyloxy group at any bond in the alkyl group, for example O—CO—CH₃, O—CO—C₂H₅, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy or 1,2-dimethylpropylcarbonyloxy.

The term “C₁-C₆-alkylthio “(C₁-C₆-alkylsulfanyl: C₁-C₆-alkyl-S—)” as used herein refers to a straight-chain or branched saturated alkyl group having 1 to 6 carbon atoms (as mentioned above) which is attached via a sulfur atom, for example C₁-C₄-alkylthio such as methylthio, ethylthio, propylthio, 1-methylethylthio, butylthio, 1-methylpropylthio, 2-methylpropylthio, 1,1-dimethylethylthio, n-pentylthiocarbonyl, 1-methylbutylthio, 2-methylbutylthio, 3-methylbutylthio, 2,2-dimethylpropylthio, 1-ethylpropylthio, n-hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutythio, 2,2-dimethylbutylthio, 2,3-dimethylbutylthio, 3,3-dimethylbutylthio, 1-ethylbutlthio, 2-ethylbutylthio, 1,1,2-trimethylpropylthio, 1,2,2-trimethylpropylthio, 1-ethyl-1-methylpropylthio or 1-ethyl-2-methylpropylthio.

The term “(C₁-C₆-alkylthio)carbonyl” as used herein refers to a straight-chain or branched alkthio group (as mentioned above) having 1 to 6 carbon atoms attached via the carbon atom of the carbonyl group. Examples include C(O)SCH₃, C(OSC₂H₅, C(O)—SCH₂—C₂H₅, C(O)SCH(CH₃)₂, n-butylthiocarbonyl, C(O)SCH(CH₃)—C₂H₅, C(O)SCH₂CH(CH₃)₂, C(O)SC(CH₃)₃, n-pentylthiocarbonyl, 1-methylbutylthiocarbonyl, 2-methylbutylthiocarbonyl, 3-methylbutylthiocarbonyl, 2,2-dimethylpropylthiocarbonyl, 1-ethylpropylthiocarbonyl, n-hexylthiocarbonyl, 1,1-dimethylpropylthiocarbonyl, 1,2-dimethylpropylthiocarbonyl, 1-methylpentylthiocarbonyl, 2-methylpentylthiocarbonyl, 3-methylpentylthiocarbonyl, 4-methylpentylthiocarbonyl, 1,1-dimethylbutylthiocarbonyl, 1,2-dimethylbutylthiocarbonyl, 1,3-dimethylbutythiocarbonyl, 2,2-dimethyl butylthiocarbonyl, 2,3-dimethylbutylthiocarbonyl, 3,3-dimethylbutylthiocarbonyl, 1-ethylbutlthioycarbonyl, 2-ethylbutylthiocarbonyl, 1,1,2-trimethylpropylthiocarbonyl, 1,2,2-trimethylpropylthiocarbonyl, 1-ethyl-1-methylpropylthiocarbonyl or 1-ethyl-2-methylpropylthiocarbonyl.

The term “C₁-C₆-alkylsulfinyl” (C₁-C₆-alkylsulfoxyl: C₁-C₆-alkyl-S(═O)—), as used herein refers to a straight-chain or branched saturated alkyl group (as mentioned above) having 1 to 6 carbon atoms bonded through the sulfur atom of the sulfinyl group at any position in the alkyl group, for example S(O)CH₃, S(O)C₂H₅, n-propylsulfinyl, 1-methylethylsulfinyl, n-butylsulfinyl, 1-methylpropylsulfinyl, 2-methylpropylsulfinyl, 1,1-dimethylethylsulfinyl, n-pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, n-hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The term “C₁-C₆-alkylamino” refers to a secondary amino group carrying one alkyl group as defined above, e.g. methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino.

The term “di(C₁-C₆-alkyl)amino)” refers to a tertiary amino group carrying two alkyl radicals as defined above, e.g. dimethylamino, diethylamino, di-n-propylamino, diisopropylamino, N-ethyl-N-methylamino, N-(n-propyl)-N-methylamino, N-(isopropyl)-N-methylamino, N-(n-butyl)-N-methylamino, N-(n-pentyl)-N-methylamino, N-(2-butyl)-N-methylamino, N-(isobutyl)-N-methylamino, N-(n-pentyl)-N-methylamino, N-(n-propyl)-N-ethylamino, N-(isopropyl)-N-ethylamino, N-(n-butyl)-N-ethylamino, N-(n-pentyl)-N-ethylamino, N-(2-butyl)-N-ethylamino, N-(isobutyl)-N-ethylamino or N-(n-pentyl)-N-ethylamino.

The term “C₁-C₆-alkylsulfonyl” (C₁-C₆-alkyl-S(═O)₂—) as used herein refers to a straight-chain or branched saturated alkyl group having 1 to 6 carbon atoms (as mentioned above) which is bonded via the sulfur atom of the sulfonyl group at any position in the alkyl group, for example SO₂—CH₃, SO₂—C₂H₅, n-propylsulfonyl, SO₂—CH(CH₃)₂, n-butylsulfonyl, 1-methylpropylsulfonyl, 2-methylpropylsulfonyl, SO₂—C(CH₃)₃, n-pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, n-hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The term “C₂-C₆-alkenyl” as used herein and in the alkenyl moieties of C₂-C₆-alkenyloxy, C₂-C₆-alkenylamino, C₂-C₆-alkenylthio, C₂-C₆-alkenylsulfonyl, (C₂-C₆-alkenyl) carbonyl, (C₂-C₆-alkenyloxy)carbonyl and (C₂-C₆-alkenyl)carbonyloxy refers to a straight-chain or branched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term, “C₂-C₆-alkenyloxy” as used herein refers to a straight-chain or branched alkenyl group having 2 to 6 carbon atoms (as mentioned above) which is attached via an oxygen atom, such as vinyloxy, allyloxy(propen-3-yloxy), methallyloxy, buten-4-yloxy, etc.

The term “C₂-C₆-alkenylthio” as used herein refers to a straight-chain or branched alkenyl group having 2 to 6 carbon atoms (as mentioned above) which is attached via a sulfur atom, for example vinylsulfanyl, allylsulfanyl(propen-3-ylthio), methallylsufanyl, buten-4-ylsulfanyl, etc.

The term “C₂-C₆-alkenylamino” as used herein refers to a straight-chain or branched alkenyl group having 2 to 6 carbon atoms (as mentioned above) which is attached via a sulfur atom, for example vinylamino, allylamino(propen-3-ylamino), methallylamino, buten-4-ylamino, etc.

The term “C₂-C₆-alkenylsulfonyl” as used herein refers to a straight-chain or branched alkenyl group having 2 to 6 carbon atoms (as mentioned above) which is attached via a sulfonyl (SO₂) group, for example vinylsulfonyl, allylsulfonyl(propen-3-ylsulfonyl), methallylsufonyl, buten-4-ylsulfonyl, etc.

The term “C₂-C₆-alkynyl” as used herein and in the alkynyl moieties of C₂-C₆-alkynyloxy, C₂-C₆-alkynylamino, C₂-C₆-alkynylthio, C₂-C₆-alkynylsulfonyl, C₂-C₆-alkynylcarbonyl, C₂-C₆-al kynyloxycarbonyl and C₁-C₆-alkynylcarbonyloxy refers to a straight-chain or branched unsaturated hydrocarbon group having 2 to 6 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl and the like.

The term, “C₂-C₆-alkynyloxy” as used herein refers to a straight-chain or branched alkynyl group having 2 to 6 carbon atoms (as mentioned above) which is attached via an oxygen atom, such as propargyloxy(propyn-3-yloxy), butyn-3-yloxy, and butyn-4-yloxy.

The term “C₂-C₆-alkynylthio” as used herein refers to a straight-chain or branched alkynyl group having 2 to 6 carbon atoms (as mentioned above) which is attached via a sulfur atom, such as propargylsulfanyl(propyn-3-ylthio), butyn-3-ylsufanyl and butyn-4-ylsulfanyl.

The term “C₂-C₆-alkynylamino” as used herein refers to a straight-chain or branched alkynyl group having 2 to 6 carbon atoms (as mentioned above) which is attached via a sulfur atom, such as propargylamino(propyn-3-ylamino), butyn-3-amino, and butyn-4-ylamino.

The term “C₂-C₆-alkynylsulfonyl” as used herein refers to a straight-chain or branched alkynyl group having 2 to 6 carbon atoms (as mentioned above) which is attached via a sulfonyl (SO₂) group, such as propargylsulfonyl(propin-3-yltsulfonyl), butin-3-ylsufonyl and butin-4-ylsulfonyl.

The term “C₃-C₁₀-cycloalkyl” as used herein refers to a mono- or bi- or polycyclic hydrocarbon radical having 3 to 8 carbon atoms, in particular 3 to 6 carbon atoms. Examples of monocyclic radicals comprise cyclopropyl, cyclobutyl, cyclopentyl, cyclohexyl, cycloheptyl, cyclooctyl, cyclononyl and cyclodecyl. Examples of bicyclic radicals comprise bicyclo[2.2.1]heptyl, bicyclo[3.1.1]heptyl, bicyclo[2.2.2]octyl and bicyclo[3.2.1]octyl.

The term “hetaryl” as used herein refers to a monocyclic heteroaromatic radical which has 5 or 6 ring members, which may comprise a fused 5, 6 or 7 membered ring thus having a total number of ring members from 8 to 10, wherein in each case 1, 2, 3 or 4 of these ring members are heteroatoms selected, independently from each other, from the group consisting of oxygen, nitrogen and sulfur. The heterocyclic radical may be attached to the remainder of the molecule via a carbon ring member or via a nitrogen ring member. The fused ring comprises C₅-C₇-cycloalkyl, C₅-C₇-cycloalkenyl, or 5 to 7 membered heterocyclyl and phenyl.

Examples for monocyclic 5- to 6-membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

Examples for 5- to 6-membered heteroaromatic rings carrying a fused phenyl ring are quinolinyl, isoquinolinyl, indolyl, indolizinyl, isoindolyl, indazolyl, benzofuryl, benzthienyl, benzo[b]thiazolyl, benzoxazolyl, benzthiazolyl, benzoxazolyl, and benzimidazolyl. Examples for 5- to 6-membered heteroaromatic rings carrying a fused cycloalkenyl ring are dihydroindolyl, dihydroindolizinyl, dihydroisoindolyl, dihydrochinolinyl, dihydroisochinolinyl, chromenyl, chromanyl and the like.

The term “5 or 6-membered heterocyclic ring” comprises heteroaromatic rings as defined above and nonaromatic saturated or partially unsaturated heterocyclic rings having 5 or 6 ring members. Examples for non-aromatic rings include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, dioxanyl, thiopyranyl, dihydrothiopyranyl, tetrahydrothiopyranyl, morpholinyl, thiazinyl and the like.

The term “5-, 6- or 7-membered carbocycle” comprises monocyclic aromatic rings and nonaromatic saturated or partially unsaturated carbocyclic rings having 5, 6 or 7 ring members. Examples for non-aromatic rings include cyclopentyl, cyclopentenyl, cyclopentadienyl, cyclohexyl, cyclohexenyl, cyclohexadienyl, cycloheptyl, cycloheptenyl, cycloheptadienyl and the like.

The term “linear (C₁-C₆)-alkandiyl” as used herein refers to methylendiyl, ethane-1,2-diyl, propane-1,3-diyl, butane-1,4-diyl, pentane-1,5-diyl, hexane-1,6-diyl.

One embodiment of the invention relates to compounds of the general formulae (I.a) or (I.b), wherein the variable A is selected from oxygen, NR⁷, sulfur, S(O) or S(O)₂.

Another embodiment relates to compounds of the general formulae (I.a) or (I.b), wherein the variable A is —C(R^{6a})(R^{6b})—. Amongst these, a particular embodiment relates to compounds (I.a) and (I.b), wherein A is C(O). Yet another particular embodiment relates to compounds, wherein at least one, preferrably both radicals R^{6a} and R^{6b} are hydrogen.

Preference is also given to compounds wherein R^{6a} and R^{6b} if different from hydrogen are selected from the group consisting of hydroxy, amino, C₁-C₆-alkyl, C₁-C₆-haloalkyl, C₁-C₆-alkoxy, C₁-C₆-alkylamino, di-(C₁-C₆-alkyl)amino, wherein the carbon atoms in the last 5 mentioned radicals may be unsubstituted or may carry any combination of 1, 2 or 3 radicals R^{a6}, C₃-C₆-cycloalkyl, phenyl or benzyl, each of the last three mentioned radicals may be unsubstituted or may carry any combination of 1, 2, 3, 4 or 5 radicals R^{b6}, or R^{6a} together with R^{6b} is ═O, ═NR^{c} or ═CR^{d}R^{e}.

One embodiment of the invention relates to compounds of the general formulae (I.a) or (I.b), wherein the variable B is a single bond.

Yet another embodiment relates to compounds of the general formulae (I.a) or (I.b), wherein B is CH₂ and A is different from —C(R^{6a})(R^{6b})—.

Preferred are compounds of the formulae (I.a) and (I.b) wherein the variables R¹, R^{2a} or R^{2b}, R^{3a}, R^{3b}, R^{3c}, R^{3d}, R^{4a}, R^{4b}, R^{z1} and X independently of one another or more preferably in combination have the meanings given below.

R¹ is preferably selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₃-C₆-cycloalkyl, phenyl or benzyl, phenoxycarbonyl, 5 or 6 membered hetaryl and 5 or 6 membered hetarylmethyl each of the six last mentioned radicals may be unsubstituted or may carry any combination of 1, 2, 3, 4 or 5 radicals R^{b1}.

Preference is given to compounds wherein R¹ is hydrogen. However, preference is also given to compounds of general formulae (I.a) or (I.b), wherein R¹ is different from hydrogen.

If R¹ is different from hydrogen, preference is given to those compounds of general formulae (I.a) and (I.b), wherein R¹ is selected from the group consisting of C₁-C₆-alkyl, C₁-C₆-haloalkyl, C₂-C₆-alkenyl, C₂-C₆-alkynyl, phenyl, benzyl, 5 or 6 membered hetaryl and 5 or 6 membered hetarylmethyl each of the last four mentioned radicals may be unsubstituted or may carry any combination of 1, 2 or 3 R^{b1}.

The variables R^{2a} or R^{2b} in formulae (I.a) or (I.b), respectively, are preferably selected from the group consisting of hydrogen, C₁-C₄-alkyl, formyl, CN, C(S)NR^{a}R^{b}, C₁-C₆-alkylcarbonyl, C₁-C₄-haloalkylcarbonyl, C₁-C₆-alkoxycarbonyl, C₁-C₄-alkoxy-C₁-C₄-alkoxycarbonyl, C₁-C₆-alkylthiocarbonyl, benzoyl, 5 or 6 membered hetarylcarbonyl, each of the last two mentioned radicals may be unsubstituted or may carry any combination of 1, 2 or 3 R^{b2}. More preferably R^{2a} or R^{2b} is hydrogen. In another preferred embodiment R^{2a} and R^{2b} are selected from the group consisting of oxazolyl, thiazolyl and imidazolyl.

In another preferred embodiment of the invention the radicals R¹ and R^{2b} together form a bridging bivalent carbonyl group C(O).

Among compounds of general formulae (I.a) or (I.b) preference is given to compounds, wherein each of the radicals R^{3a}, R^{3b}, R^{3c} and R^{3d} is hydrogen.

Preference is given to compounds of general formulae (I.a) and (I.b) wherein the radicals R^{4a} and R^{4b} are selected from the group consisting of hydrogen, halogen, C₁-C₆-alkyl, C₂-C₆-alkenyl, C₂-C₆-alkynyl, phenyl, 5 or 6 membered hetaryl, 5 or 6 membered hetarylmethyl and benzyl wherein the four last mentioned radicals may be unsubstituted or may carry any combination of 1, 2, 3, 4 or 5 radicals R^{b4}.

In a preferred embodiment of the present invention one or both of the radicals R^{4a} and R^{4b} are different from hydrogen.

Preferably R^{4a} is different from hydrogen. Particular preference is given to compounds of general formulae (I.a) or (I.b), wherein R^{4a} is selected from the group consisting of hydrogen, halogen, C₁-C₆-alkyl, C₂-C₆-alkenyl, C₂-C₆-alkynyl and benzyl which may be unsubstituted or may carry any combination of 1, 2, 3, 4 or 5 radicals R^{b4}.

In another preferred embodiment of the invention the radicals R^{2a} and R^{4a} together form a bridging bivalent radical. Preferred examples of such bridging radicals are C(O)—CH₂, C(S)—CH₂, CH₂—CH₂, S(O)₂—CH₂, S(O)—CH₂, C(O)—O, C(S)—O, S(O)₂—O, S(O)—O, C(O)—NH, C(S)—NH, S(O)₂—NH, S(O)—NH, wherein the last eight mentioned radicals are attached to the nitrogen atom via the carbonyl group, the thiocarbonyl group or the sulphur atom respectively. More preferred examples of such radicals are C(O)—CH₂, C(S)—CH₂, CH₂—CH₂, S(O)₂—CH₂, S(O)—CH₂.

Furthermore, preference is given to compounds of general formulae (I.a) or (I.b), wherein R^{4b} is hydrogen or C₁-C₆-alkyl.

Likewise preferred are compounds of general formulae (I.a) or (I.b), wherein R^{4a} together with R^{4b} form a radical ═O, ═NR^{c} or ═CR^{d}R^{e}. Preferably radicals R^{c}, R^{d} and R^{e} are selected from the group consisting of hydrogen, C₁-C₆-alkyl, phenyl, hydroxy, C₁-C₆-alkoxy, C₁-C₆-alkylamino, di-(C₁-C₆-alkyl)amino.

Preferred are compounds of general formulae (I.a) or (I.b) wherein the phenyl moiety carries 1, 2, 3, 4 or 5 radicals R^{z1}, which are independently of each other selected from the group consisting of halogen, C₁-C₆-alkyl, C₁-C₆-haloalkyl, C₁-C₆-alkoxy, C₁-C₆-haloalkoxy, C₁-C₆-alkylthio and C₁-C₆-haloalkylthio.

In one preferred embodiment of the present invention the variable X in compounds of the general formulae (I.a) or (I.b) is sulfur. In another preferred embodiment X is O. In yet another preferred embodiment X is NR⁵.

Examples of preferred embodiements of the present invention are illustrated by formulae (I.a.1), (I.b.1), (I.a.2), (I.b.2), (I.a.3), (I.b.3), (I.a.4), (I.b.4), (I.a.5), (I.b.6), (I.b.6), (I.a.7), (I.b.7), (I.a.8), (I.b.8), (I.a.9), (I.b.9), (I.a.10), (I.b.10), (I.a.11), (I.b.11), (I.a.12), (I.b.12), (I.a.13), (I.b.13), (I.a.14), (I.b.14), (I.a.15) and (I.a.16).

In formulae (I.a.11), (I.b.11), (I.a.12) and (I.b.12) R¹ is different from hydrogen. In formulae (I.a.1), (I.b.1), (I.a.2) and (I.b.2) at least one of the radicals R^{4a} and R^{4b} is different from hydrogen. Furthermore in formulae (I.a.13), (I.b.13), (I.a.14) and (I.b.14) R¹ and at least one of the radicals R^{4a} and R^{4b} are different from hydrogen.

If at least one radical R^{4a}, R^{4b} or R^{6a} different from hydrogen is present, the structures depicted represent any possible combination of cisoide or transoide positions of said radical relativ to the amino group and to one another.

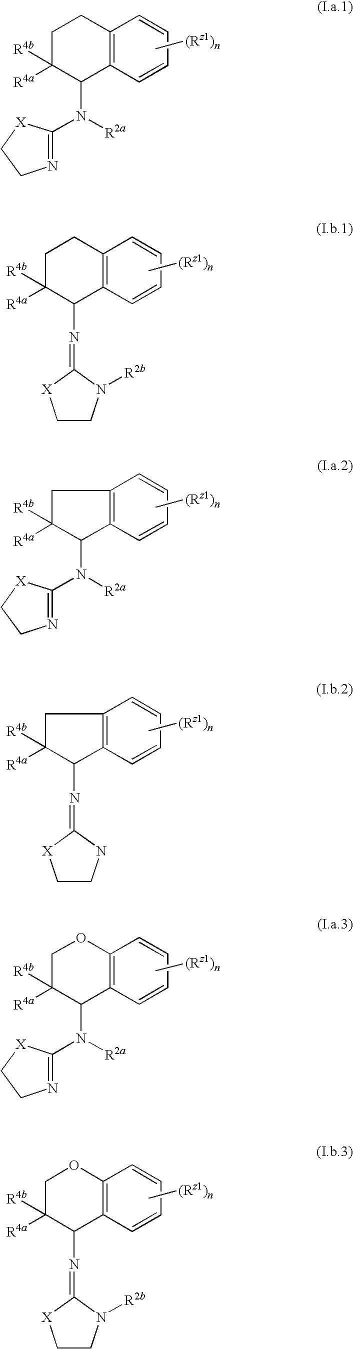

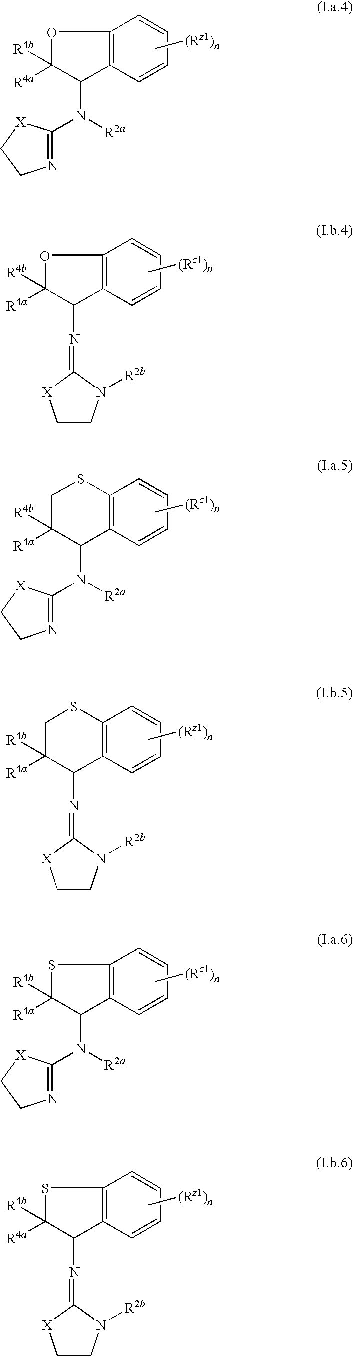

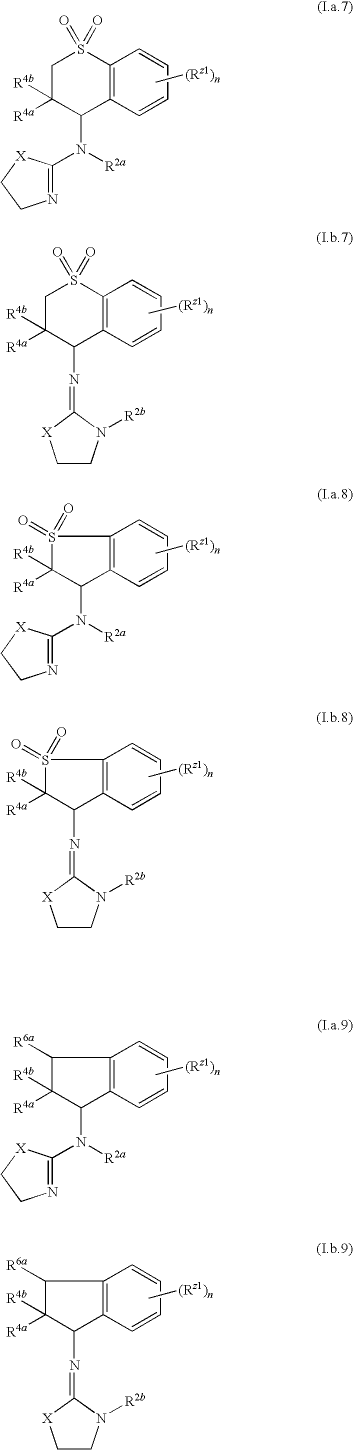

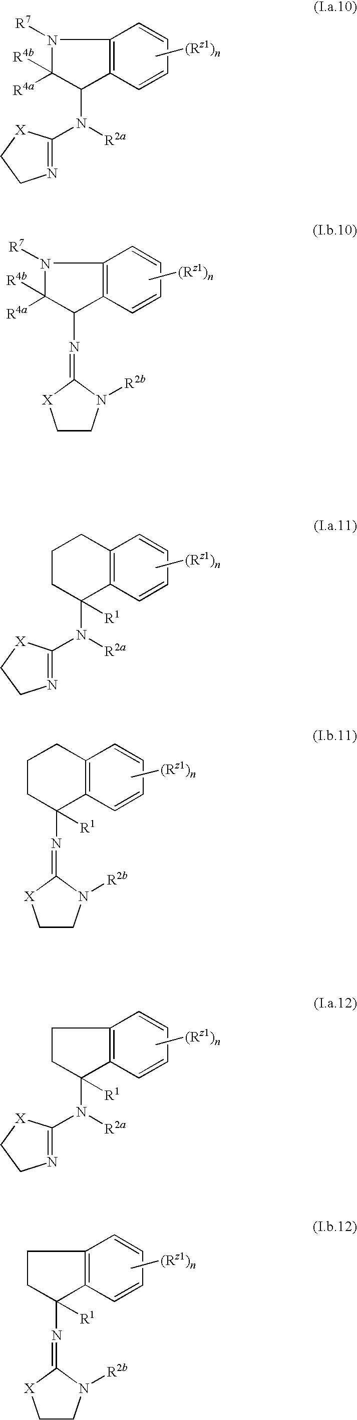

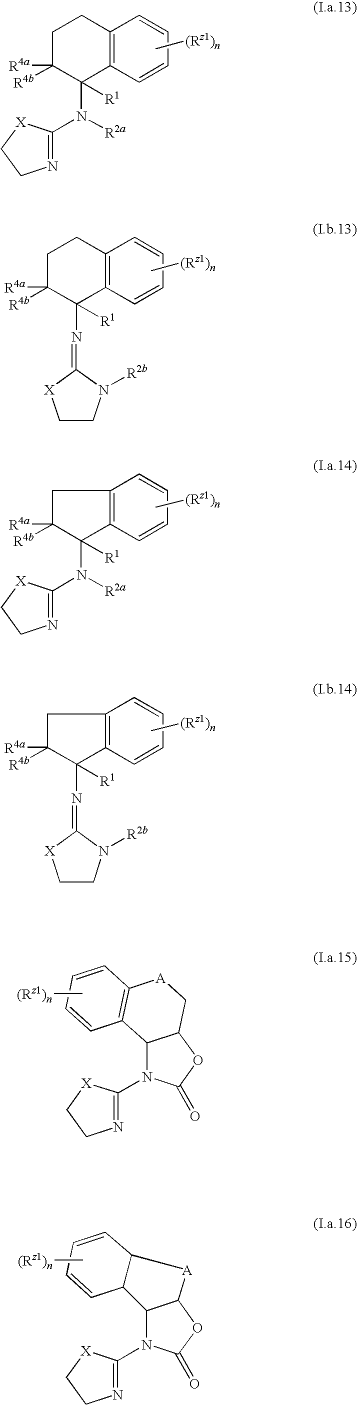

Examples for such preferred compounds are given in tables 1 to 640.

TABLE 1
Compounds of the formulae (I.a) or (I.b) and their mixtures, wherein
X is S, A is CH₂, R¹ is hydrogen, R^{2a} or R^{2b} is hydrogen, R^{3a}, R^{3b},
R^{3c} and R^{3d} are hydrogen and wherein B, (R^{z1})ₙ, R^{4a} and R^{4b} have the
meanings given in any of lines 145 to 1296 of table A.

| | |
|---|---|
| 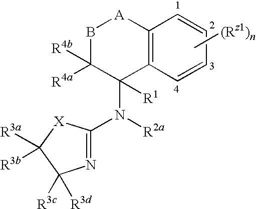 | (I.a) |
| 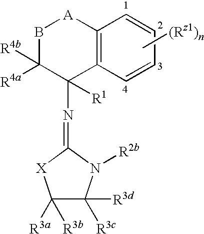 | (I.b) |

TABLE A

| | B | (R^{z1})ₙ | R^{4a} | R^{4b} |
|---|---|---|---|---|
| 1 | — | 1-CH₃ | H | H |
| 2 | — | 2-CH₃ | H | H |
| 3 | — | 3-CH₃ | H | H |
| 4 | — | 4-CH₃ | H | H |
| 5 | — | 1-CH₃, 3-CH₃ | H | H |
| 6 | — | 1-CH₃, 3-CH₃, 4-CH₃ | H | H |
| 7 | — | 3-CH₃, 4-CH₃ | H | H |
| 8 | — | 1-Cl | H | H |
| 9 | — | 2-Cl | H | H |
| 10 | — | 3-Cl | H | H |
| 11 | — | 4-Cl | H | H |
| 12 | — | 1-Cl, 3-Cl | H | H |
| 13 | — | 1-Cl, 3-Cl, 4-Cl | H | H |
| 14 | — | 3-Cl, 4-Cl | H | H |
| 15 | — | 1-F | H | H |
| 16 | — | 2-F | H | H |
| 17 | — | 3-F | H | H |
| 18 | — | 4-F | H | H |
| 19 | — | 1-F, 3-F | H | H |
| 20 | — | 1-F, 3-F, 4-F | H | H |
| 21 | — | 3-F, 4-F | H | H |
| 22 | — | 1-Br | H | H |
| 23 | — | 2-Br | H | H |
| 24 | — | 3-Br | H | H |
| 25 | — | 4-Br | H | H |
| 26 | — | 1-Br, 3-Br | H | H |
| 27 | — | 1-Br, 3-Br, 4-Br | H | H |
| 28 | — | 3-Br, 4-Br | H | H |
| 29 | — | 1-CF₃ | H | H |
| 30 | — | 2-CF₃ | H | H |
| 31 | — | 3-CF₃ | H | H |
| 32 | — | 4-CF₃ | H | H |
| 33 | — | 1CF₃, 3-CF₃ | H | H |
| 34 | — | 1-CF₃, 3-CF₃, 4-CF₃ | H | H |
| 35 | — | 3-CF₃, 4-CF3 | H | H |
| 36 | — | 1-OCH₃ | H | H |
| 37 | — | 2-OCH₃ | H | H |
| 38 | — | 3-OCH₃ | H | H |
| 39 | — | 4-OCH₃ | H | H |
| 40 | — | 1-OCH₃, 3-OCH₃ | H | H |
| 41 | — | 1-OCH₃, 3-OCH₃, 4-OCH₃ | H | H |
| 42 | — | 3,4-OCH₃, OCH₃ | H | H |
| 43 | — | 1-CH₃, 3-Cl | H | H |
| 44 | — | 1-Cl, 3-CH₃ | H | H |
| 45 | — | 1-CH₃, 3-F | H | H |
| 46 | — | 1-F, 3-CH₃ | H | H |
| 47 | — | 1-CH₃, 3-Br | H | H |
| 48 | — | 1-Br, 3-CH₃ | H | H |
| 49 | — | 1-CH₃, 3-CF₃ | H | H |
| 50 | — | 1-CF₃, 3-CH₃ | H | H |
| 51 | — | 1-CH₃, 3OCH₃ | H | H |
| 52 | — | 1-OCH₃, 3-CH₃ | H | H |
| 53 | — | 1-Cl, 3-F | H | H |
| 54 | — | 1-F, 3-Cl | H | H |
| 55 | — | 1-Cl, 3-Br | H | H |
| 56 | — | 1-Br, 3-Cl | H | H |
| 57 | — | 1-Cl, 3-CF3 | H | H |
| 58 | — | 11-CF₃, 3-Cl | H | H |
| 59 | — | 1-Cl, 3-OCH₃ | H | H |
| 60 | — | 1-OCH₃, 3-Cl | H | H |
| 61 | — | 1-F, 3-Br | H | H |
| 62 | — | 1-Br, 3-F | H | H |
| 63 | — | 1-F, 3-CF₃ | H | H |
| 64 | — | 1-CF₃, 3-F | H | H |
| 65 | — | 1-F, 3-OCH₃ | H | H |
| 66 | — | 1-OCH₃, 3-F | H | H |
| 67 | — | 1-Br, 3-CF3 | H | H |
| 68 | — | 1-CF₃, 3-Br | H | H |
| 69 | — | 1-Br, 3-OCH₃ | H | H |
| 70 | — | 1-OCH₃, 3-Br | H | H |
| 71 | — | 1-CF₃, 3-OCH₃ | H | H |
| 72 | — | 1-OCH₃, 3-CF₃ | H | H |
| 73 | CH₂ | 1-CH₃ | H | H |
| 74 | CH₂ | 2-CH₃ | H | H |
| 75 | CH₂ | 3-CH₃ | H | H |
| 76 | CH₂ | 4-CH₃ | H | H |
| 77 | CH₂ | 1-CH₃, 3-CH₃ | H | H |
| 78 | CH₂ | 1-CH₃, 3-CH₃, 4-CH₃ | H | H |
| 79 | CH₂ | 3-CH₃, 4-CH₃ | H | H |
| 80 | CH₂ | 1-Cl | H | H |
| 81 | CH₂ | 2-Cl | H | H |
| 82 | CH₂ | 3-Cl | H | H |
| 83 | CH₂ | 4-Cl | H | H |
| 84 | CH₂ | 1-Cl, 3-Cl | H | H |
| 85 | CH₂ | 1-Cl, 3-Cl, 4-Cl | H | H |
| 86 | CH₂ | 3-Cl, 4-Cl | H | H |
| 87 | CH₂ | 1-F | H | H |
| 88 | CH₂ | 2-F | H | H |
| 89 | CH₂ | 3-F | H | H |
| 90 | CH₂ | 4-F | H | H |
| 91 | CH₂ | 1-F, 3-F | H | H |
| 92 | CH₂ | 1-F, 3-F, 4-F | H | H |
| 93 | CH₂ | 3-F, 4-F | H | H |
| 94 | CH₂ | 1-Br | H | H |
| 95 | CH₂ | 2-Br | H | H |
| 96 | CH₂ | 3-Br | H | H |
| 97 | CH₂ | 4-Br | H | H |
| 98 | CH₂ | 1-Br, 3-Br | H | H |
| 99 | CH₂ | 1-Br, 3-Br, 4-Br | H | H |
| 100 | CH₂ | 3-Br, 4-Br | H | H |
| 101 | CH₂ | 1-CF₃ | H | H |
| 102 | CH₂ | 2-CF₃ | H | H |
| 103 | CH₂ | 3-CF₃ | H | H |
| 104 | CH₂ | 4-CF₃ | H | H |
| 105 | CH₂ | 1CF₃, 3-CF₃ | H | H |
| 106 | CH₂ | 1-CF₃, 3-CF₃, 4-CF₃ | H | H |
| 107 | CH₂ | 3-CF₃, 4-CF3 | H | H |
| 108 | CH₂ | 1-OCH₃ | H | H |
| 109 | CH₂ | 2-OCH₃ | H | H |
| 110 | CH₂ | 3-OCH₃ | H | H |
| 111 | CH₂ | 4-OCH₃ | H | H |
| 112 | CH₂ | 1-OCH₃, 3-OCH₃ | H | H |
| 113 | CH₂ | 1-OCH₃, 3-OCH₃, 4-OCH₃ | H | H |
| 114 | CH₂ | 3,4-OCH₃, OCH₃ | H | H |
| 115 | CH₂ | 1-CH₃, 3-Cl | H | H |
| 116 | CH₂ | 1-Cl, 3-CH₃ | H | H |
| 117 | CH₂ | 1-CH₃, 3-F | H | H |
| 118 | CH₂ | 1-F, 3-CH₃ | H | H |
| 119 | CH₂ | 1-CH₃, 3-Br | H | H |
| 120 | CH₂ | 1-Br, 3-CH₃ | H | H |
| 121 | CH₂ | 1-CH₃, 3-CF₃ | H | H |
| 122 | CH₂ | 1-CF₃, 3-CH₃ | H | H |
| 123 | CH₂ | 1-CH₃, 3OCH₃ | H | H |
| 124 | CH₂ | 1-OCH₃, 3-CH₃ | H | H |
| 125 | CH₂ | 1-Cl, 3-F | H | H |
| 126 | CH₂ | 1-F, 3-Cl | H | H |
| 127 | CH₂ | 1-Cl, 3-Br | H | H |
| 128 | CH₂ | 1-Br, 3-Cl | H | H |
| 129 | CH₂ | 1-Cl, 3-CF3 | H | H |
| 130 | CH₂ | 11-CF₃, 3-Cl | H | H |
| 131 | CH₂ | 1-Cl, 3-OCH₃ | H | H |
| 132 | CH₂ | 1-OCH₃, 3-Cl | H | H |
| 133 | CH₂ | 1-F, 3-Br | H | H |
| 134 | CH₂ | 1-Br, 3-F | H | H |
| 135 | CH₂ | 1-F, 3-CF₃ | H | H |
| 136 | CH₂ | 1-CF₃, 3-F | H | H |
| 137 | CH₂ | 1-F, 3-OCH₃ | H | H |
| 138 | CH₂ | 1-OCH₃, 3-F | H | H |
| 139 | CH₂ | 1-Br, 3-CF3 | H | H |
| 140 | CH₂ | 1-CF₃, 3-Br | H | H |
| 141 | CH₂ | 1-Br, 3-OCH₃ | H | H |
| 142 | CH₂ | 1-OCH₃, 3-Br | H | H |
| 143 | CH₂ | 1-CF₃, 3-OCH₃ | H | H |
| 144 | CH₂ | 1-OCH₃, 3-CF₃ | H | H |
| 145 | — | 1-CH₃ | CH₃ | H |
| 146 | — | 2-CH₃ | CH₃ | H |
| 147 | — | 3-CH₃ | CH₃ | H |
| 148 | — | 4-CH₃ | CH₃ | H |
| 149 | — | 1-CH₃, 3-CH₃ | CH₃ | H |
| 150 | — | 1-CH₃, 3-CH₃, 4-CH₃ | CH₃ | H |
| 151 | — | 3-CH₃, 4-CH₃ | CH₃ | H |
| 152 | — | 1-Cl | CH₃ | H |
| 153 | — | 2-Cl | CH₃ | H |
| 154 | — | 3-Cl | CH₃ | H |
| 155 | — | 4-Cl | CH₃ | H |
| 156 | — | 1-Cl, 3-Cl | CH₃ | H |
| 157 | — | 1-Cl, 3-Cl, 4-Cl | CH₃ | H |
| 158 | — | 3-Cl, 4-Cl | CH₃ | H |
| 159 | — | 1-F | CH₃ | H |
| 160 | — | 2-F | CH₃ | H |
| 161 | — | 3-F | CH₃ | H |
| 162 | — | 4-F | CH₃ | H |
| 163 | — | 1-F, 3-F | CH₃ | H |
| 164 | — | 1-F, 3-F, 4-F | CH₃ | H |
| 165 | — | 3-F, 4-F | CH₃ | H |
| 166 | — | 1-Br | CH₃ | H |
| 167 | — | 2-Br | CH₃ | H |
| 168 | — | 3-Br | CH₃ | H |
| 169 | — | 4-Br | CH₃ | H |
| 170 | — | 1-Br, 3-Br | CH₃ | H |
| 171 | — | 1-Br, 3-Br, 4-Br | CH₃ | H |
| 172 | — | 3-Br, 4-Br | CH₃ | H |
| 173 | — | 1-CF₃ | CH₃ | H |
| 174 | — | 2-CF₃ | CH₃ | H |
| 175 | — | 3-CF₃ | CH₃ | H |
| 176 | — | 4-CF₃ | CH₃ | H |
| 177 | — | 1CF₃, 3-CF₃ | CH₃ | H |
| 178 | — | 1-CF₃, 3-CF₃, 4-CF₃ | CH₃ | H |
| 179 | — | 3-CF₃, 4-CF3 | CH₃ | H |
| 180 | — | 1-OCH₃ | CH₃ | H |
| 181 | — | 2-OCH₃ | CH₃ | H |
| 182 | — | 3-OCH₃ | CH₃ | H |
| 183 | — | 4-OCH₃ | CH₃ | H |
| 184 | — | 1-OCH₃, 3-OCH₃ | CH₃ | H |
| 185 | — | 1-OCH₃, 3-OCH₃, 4-OCH₃ | CH₃ | H |
| 186 | — | 3,4-OCH₃, OCH₃ | CH₃ | H |
| 187 | — | 1-CH₃, 3-Cl | CH₃ | H |
| 188 | — | 1-Cl, 3-CH₃ | CH₃ | H |
| 189 | — | 1-CH₃, 3-F | CH₃ | H |
| 190 | — | 1-F, 3-CH₃ | CH₃ | H |
| 191 | — | 1-CH₃, 3-Br | CH₃ | H |
| 192 | — | 1-Br, 3-CH₃ | CH₃ | H |
| 193 | — | 1-CH₃, 3-CF₃ | CH₃ | H |
| 194 | — | 1-CF₃, 3-CH₃ | CH₃ | H |
| 195 | — | 1-CH₃, 3OCH₃ | CH₃ | H |
| 196 | — | 1-OCH₃, 3-CH₃ | CH₃ | H |
| 197 | — | 1-Cl, 3-F | CH₃ | H |
| 198 | — | 1-F, 3-Cl | CH₃ | H |
| 199 | — | 1-Cl, 3-Br | CH₃ | H |
| 200 | — | 1-Br, 3-Cl | CH₃ | H |
| 201 | — | 1-Cl, 3-CF3 | CH₃ | H |
| 202 | — | 11-CF₃, 3-Cl | CH₃ | H |
| 203 | — | 1-Cl, 3-OCH₃ | CH₃ | H |
| 204 | — | 1-OCH₃, 3-Cl | CH₃ | H |
| 205 | — | 1-F, 3-Br | CH₃ | H |
| 206 | — | 1-Br, 3-F | CH₃ | H |
| 207 | — | 1-F, 3-CF₃ | CH₃ | H |
| 208 | — | 1-CF₃, 3-F | CH₃ | H |
| 209 | — | 1-F, 3-OCH₃ | CH₃ | H |
| 210 | — | 1-OCH₃, 3-F | CH₃ | H |
| 211 | — | 1-Br, 3-CF3 | CH₃ | H |
| 212 | — | 1-CF₃, 3-Br | CH₃ | H |
| 213 | — | 1-Br, 3-OCH₃ | CH₃ | H |
| 214 | — | 1-OCH₃, 3-Br | CH₃ | H |
| 215 | — | 1-CF₃, 3-OCH₃ | CH₃ | H |
| 216 | — | 1-OCH₃, 3-CF₃ | CH₃ | H |
| 217 | CH₂ | 1-CH₃ | CH₃ | H |
| 218 | CH₂ | 2-CH₃ | CH₃ | H |
| 219 | CH₂ | 3-CH₃ | CH₃ | H |
| 220 | CH₂ | 4-CH₃ | CH₃ | H |
| 221 | CH₂ | 1-CH₃, 3-CH₃ | CH₃ | H |
| 222 | CH₂ | 1-CH₃, 3-CH₃, 4-CH₃ | CH₃ | H |
| 223 | CH₂ | 3-CH₃, 4-CH₃ | CH₃ | H |
| 224 | CH₂ | 1-Cl | CH₃ | H |
| 225 | CH₂ | 2-Cl | CH₃ | H |
| 226 | CH₂ | 3-Cl | CH₃ | H |
| 227 | CH₂ | 4-Cl | CH₃ | H |
| 228 | CH₂ | 1-Cl, 3-Cl | CH₃ | H |
| 229 | CH₂ | 1-Cl, 3-Cl, 4-Cl | CH₃ | H |
| 230 | CH₂ | 3-Cl, 4-Cl | CH₃ | H |
| 231 | CH₂ | 1-F | CH₃ | H |
| 232 | CH₂ | 2-F | CH₃ | H |
| 233 | CH₂ | 3-F | CH₃ | H |
| 234 | CH₂ | 4-F | CH₃ | H |
| 235 | CH₂ | 1-F, 3-F | CH₃ | H |
| 236 | CH₂ | 1-F, 3-F, 4-F | CH₃ | H |
| 237 | CH₂ | 3-F, 4-F | CH₃ | H |
| 238 | CH₂ | 1-Br | CH₃ | H |
| 239 | CH₂ | 2-Br | CH₃ | H |
| 240 | CH₂ | 3-Br | CH₃ | H |
| 241 | CH₂ | 4-Br | CH₃ | H |
| 242 | CH₂ | 1-Br, 3-Br | CH₃ | H |
| 243 | CH₂ | 1-Br, 3-Br, 4-Br | CH₃ | H |
| 244 | CH₂ | 3-Br, 4-Br | CH₃ | H |
| 245 | CH₂ | 1-CF₃ | CH₃ | H |
| 246 | CH₂ | 2-CF₃ | CH₃ | H |
| 247 | CH₂ | 3-CF₃ | CH₃ | H |
| 248 | CH₂ | 4-CF₃ | CH₃ | H |
| 249 | CH₂ | 1CF₃, 3-CF₃ | CH₃ | H |
| 250 | CH₂ | 1-CF₃, 3-CF₃, 4-CF₃ | CH₃ | H |
| 251 | CH₂ | 3-CF₃, 4-CF3 | CH₃ | H |
| 252 | CH₂ | 1-OCH₃ | CH₃ | H |
| 253 | CH₂ | 2-OCH₃ | CH₃ | H |
| 254 | CH₂ | 3-OCH₃ | CH₃ | H |
| 255 | CH₂ | 4-OCH₃ | CH₃ | H |
| 256 | CH₂ | 1-OCH₃, 3-OCH₃ | CH₃ | H |
| 257 | CH₂ | 1-OCH₃, 3-OCH₃, 4-OCH₃ | CH₃ | H |
| 258 | CH₂ | 3,4-OCH₃, OCH₃ | CH₃ | H |
| 259 | CH₂ | 1-CH₃, 3-Cl | CH₃ | H |
| 260 | CH₂ | 1-Cl, 3-CH₃ | CH₃ | H |
| 261 | CH₂ | 1-CH₃, 3-F | CH₃ | H |
| 262 | CH₂ | 1-F, 3-CH₃ | CH₃ | H |
| 263 | CH₂ | 1-CH₃, 3-Br | CH₃ | H |
| 264 | CH₂ | 1-Br, 3-CH₃ | CH₃ | H |
| 265 | CH₂ | 1-CH₃, 3-CF₃ | CH₃ | H |
| 266 | CH₂ | 1-CF₃, 3-CH₃ | CH₃ | H |
| 267 | CH₂ | 1-CH₃, 3OCH₃ | CH₃ | H |
| 268 | CH₂ | 1-OCH₃, 3-CH₃ | CH₃ | H |
| 269 | CH₂ | 1-Cl, 3-F | CH₃ | H |
| 270 | CH₂ | 1-F, 3-Cl | CH₃ | H |
| 271 | CH₂ | 1-Cl, 3-Br | CH₃ | H |
| 272 | CH₂ | 1-Br, 3-Cl | CH₃ | H |
| 273 | CH₂ | 1-Cl, 3-CF3 | CH₃ | H |
| 274 | CH₂ | 11-CF₃, 3-Cl | CH₃ | H |
| 275 | CH₂ | 1-Cl, 3-OCH₃ | CH₃ | H |
| 276 | CH₂ | 1-OCH₃, 3-Cl | CH₃ | H |
| 277 | CH₂ | 1-F, 3-Br | CH₃ | H |
| 278 | CH₂ | 1-Br, 3-F | CH₃ | H |
| 279 | CH₂ | 1-F, 3-CF₃ | CH₃ | H |
| 280 | CH₂ | 1-CF₃, 3-F | CH₃ | H |
| 281 | CH₂ | 1-F, 3-OCH₃ | CH₃ | H |
| 282 | CH₂ | 1-OCH₃, 3-F | CH₃ | H |
| 283 | CH₂ | 1-Br, 3-CF3 | CH₃ | H |
| 284 | CH₂ | 1-CF₃, 3-Br | CH₃ | H |
| 285 | CH₂ | 1-Br, 3-OCH₃ | CH₃ | H |
| 286 | CH₂ | 1-OCH₃, 3-Br | CH₃ | H |
| 287 | CH₂ | 1-CF₃, 3-OCH₃ | CH₃ | H |
| 288 | CH₂ | 1-OCH₃, 3-CF₃ | CH₃ | H |
| 289 | — | 1-CH₃ | n-propenyl | H |
| 290 | — | 2-CH₃ | n-propenyl | H |
| 291 | — | 3-CH₃ | n-propenyl | H |
| 292 | — | 4-CH₃ | n-propenyl | H |
| 293 | — | 1-CH₃, 3-CH₃ | n-propenyl | H |
| 294 | — | 1-CH₃, 3-CH₃, 4-CH₃ | n-propenyl | H |
| 295 | — | 3-CH₃, 4-CH₃ | n-propenyl | H |
| 296 | — | 1-Cl | n-propenyl | H |
| 297 | — | 2-Cl | n-propenyl | H |
| 298 | — | 3-Cl | n-propenyl | H |
| 299 | — | 4-Cl | n-propenyl | H |
| 300 | — | 1-Cl, 3-Cl | n-propenyl | H |
| 301 | — | 1-Cl, 3-Cl, 4-Cl | n-propenyl | H |
| 302 | — | 3-Cl, 4-Cl | n-propenyl | H |
| 303 | — | 1-F | n-propenyl | H |
| 304 | — | 2-F | n-propenyl | H |
| 305 | — | 3-F | n-propenyl | H |
| 306 | — | 4-F | n-propenyl | H |
| 307 | — | 1-F, 3-F | n-propenyl | H |
| 308 | — | 1-F, 3-F, 4-F | n-propenyl | H |
| 309 | — | 3-F, 4-F | n-propenyl | H |
| 310 | — | 1-Br | n-propenyl | H |
| 311 | — | 2-Br | n-propenyl | H |
| 312 | — | 3-Br | n-propenyl | H |
| 313 | — | 4-Br | n-propenyl | H |
| 314 | — | 1-Br, 3-Br | n-propenyl | H |
| 315 | — | 1-Br, 3-Br, 4-Br | n-propenyl | H |
| 316 | — | 3-Br, 4-Br | n-propenyl | H |
| 317 | — | 1-CF₃ | n-propenyl | H |
| 318 | — | 2-CF₃ | n-propenyl | H |
| 319 | — | 3-CF₃ | n-propenyl | H |
| 320 | — | 4-CF₃ | n-propenyl | H |
| 321 | — | 1CF₃, 3-CF₃ | n-propenyl | H |
| 322 | — | 1-CF₃, 3-CF₃, 4-CF₃ | n-propenyl | H |
| 323 | — | 3-CF₃, 4-CF3 | n-propenyl | H |
| 324 | — | 1-OCH₃ | n-propenyl | H |
| 325 | — | 2-OCH₃ | n-propenyl | H |
| 326 | — | 3-OCH₃ | n-propenyl | H |
| 327 | — | 4-OCH₃ | n-propenyl | H |
| 328 | — | 1-OCH₃, 3-OCH₃ | n-propenyl | H |
| 329 | — | 1-OCH₃, 3-OCH₃, 4-OCH₃ | n-propenyl | H |
| 330 | — | 3,4-OCH₃, OCH₃ | n-propenyl | H |
| 331 | — | 1-CH₃, 3-Cl | n-propenyl | H |
| 332 | — | 1-Cl, 3-CH₃ | n-propenyl | H |
| 333 | — | 1-CH₃, 3-F | n-propenyl | H |
| 334 | — | 1-F, 3-CH₃ | n-propenyl | H |
| 335 | — | 1-CH₃, 3-Br | n-propenyl | H |
| 336 | — | 1-Br, 3-CH₃ | n-propenyl | H |
| 337 | — | 1-CH₃, 3-CF₃ | n-propenyl | H |
| 338 | — | 1-CF₃, 3-CH₃ | n-propenyl | H |
| 339 | — | 1-CH₃, 3OCH₃ | n-propenyl | H |
| 340 | — | 1-OCH₃, 3-CH₃ | n-propenyl | H |
| 341 | — | 1-Cl, 3-F | n-propenyl | H |
| 342 | — | 1-F, 3-Cl | n-propenyl | H |
| 343 | — | 1-Cl, 3-Br | n-propenyl | H |
| 344 | — | 1-Br, 3-Cl | n-propenyl | H |
| 345 | — | 1-Cl, 3-CF3 | n-propenyl | H |
| 346 | — | 11-CF₃, 3-Cl | n-propenyl | H |
| 347 | — | 1-Cl, 3-OCH₃ | n-propenyl | H |
| 348 | — | 1-OCH₃, 3-Cl | n-propenyl | H |
| 349 | — | 1-F, 3-Br | n-propenyl | H |
| 350 | — | 1-Br, 3-F | n-propenyl | H |
| 351 | — | 1-F, 3-CF₃ | n-propenyl | H |
| 352 | — | 1-CF₃, 3-F | n-propenyl | H |
| 353 | — | 1-F, 3-OCH₃ | n-propenyl | H |
| 354 | — | 1-OCH₃, 3-F | n-propenyl | H |
| 355 | — | 1-Br, 3-CF3 | n-propenyl | H |
| 356 | — | 1-CF₃, 3-Br | n-propenyl | H |
| 357 | — | 1-Br, 3-OCH₃ | n-propenyl | H |
| 358 | — | 1-OCH₃, 3-Br | n-propenyl | H |
| 359 | — | 1-CF₃, 3-OCH₃ | n-propenyl | H |
| 360 | — | 1-OCH₃, 3-CF₃ | n-propenyl | H |
| 361 | CH₂ | 1-CH₃ | n-propenyl | H |
| 362 | CH₂ | 2-CH₃ | n-propenyl | H |
| 363 | CH₂ | 3-CH₃ | n-propenyl | H |
| 364 | CH₂ | 4-CH₃ | n-propenyl | H |
| 365 | CH₂ | 1-CH₃, 3-CH₃ | n-propenyl | H |
| 366 | CH₂ | 1-CH₃, 3-CH₃, 4-CH₃ | n-propenyl | H |
| 367 | CH₂ | 3-CH₃, 4-CH₃ | n-propenyl | H |
| 368 | CH₂ | 1-Cl | n-propenyl | H |
| 369 | CH₂ | 2-Cl | n-propenyl | H |
| 370 | CH₂ | 3-Cl | n-propenyl | H |
| 371 | CH₂ | 4-Cl | n-propenyl | H |
| 372 | CH₂ | 1-Cl, 3-Cl | n-propenyl | H |
| 373 | CH₂ | 1-Cl, 3-Cl, 4-Cl | n-propenyl | H |
| 374 | CH₂ | 3-Cl, 4-Cl | n-propenyl | H |
| 375 | CH₂ | 1-F | n-propenyl | H |
| 376 | CH₂ | 2-F | n-propenyl | H |
| 377 | CH₂ | 3-F | n-propenyl | H |
| 378 | CH₂ | 4-F | n-propenyl | H |
| 379 | CH₂ | 1-F, 3-F | n-propenyl | H |
| 380 | CH₂ | 1-F, 3-F, 4-F | n-propenyl | H |
| 381 | CH₂ | 3-F, 4-F | n-propenyl | H |
| 382 | CH₂ | 1-Br | n-propenyl | H |
| 383 | CH₂ | 2-Br | n-propenyl | H |
| 384 | CH₂ | 3-Br | n-propenyl | H |
| 385 | CH₂ | 4-Br | n-propenyl | H |
| 386 | CH₂ | 1-Br, 3-Br | n-propenyl | H |
| 387 | CH₂ | 1-Br, 3-Br, 4-Br | n-propenyl | H |
| 388 | CH₂ | 3-Br, 4-Br | n-propenyl | H |
| 389 | CH₂ | 1-CF₃ | n-propenyl | H |
| 390 | CH₂ | 2-CF₃ | n-propenyl | H |
| 391 | CH₂ | 3-CF₃ | n-propenyl | H |
| 392 | CH₂ | 4-CF₃ | n-propenyl | H |
| 393 | CH₂ | 1CF₃, 3-CF₃ | n-propenyl | H |
| 394 | CH₂ | 1-CF₃, 3-CF₃, 4-CF₃ | n-propenyl | H |
| 395 | CH₂ | 3-CF₃, 4-CF3 | n-propenyl | H |
| 396 | CH₂ | 1-OCH₃ | n-propenyl | H |
| 397 | CH₂ | 2-OCH₃ | n-propenyl | H |
| 398 | CH₂ | 3-OCH₃ | n-propenyl | H |
| 399 | CH₂ | 4-OCH₃ | n-propenyl | H |
| 400 | CH₂ | 1-OCH₃, 3-OCH₃ | n-propenyl | H |
| 401 | CH₂ | 1-OCH₃, 3-OCH₃, 4-OCH₃ | n-propenyl | H |
| 402 | CH₂ | 3,4-OCH₃, OCH₃ | n-propenyl | H |
| 403 | CH₂ | 1-CH₃, 3-Cl | n-propenyl | H |
| 404 | CH₂ | 1-Cl, 3-CH₃ | n-propenyl | H |
| 405 | CH₂ | 1-CH₃, 3-F | n-propenyl | H |
| 406 | CH₂ | 1-F, 3-CH₃ | n-propenyl | H |
| 407 | CH₂ | 1-CH₃, 3-Br | n-propenyl | H |
| 408 | CH₂ | 1-Br, 3-CH₃ | n-propenyl | H |
| 409 | CH₂ | 1-CH₃, 3-CF₃ | n-propenyl | H |
| 410 | CH₂ | 1-CF₃, 3-CH₃ | n-propenyl | H |
| 411 | CH₂ | 1-CH₃, 3OCH₃ | n-propenyl | H |
| 412 | CH₂ | 1-OCH₃, 3-CH₃ | n-propenyl | H |
| 413 | CH₂ | 1-Cl, 3-F | n-propenyl | H |
| 414 | CH₂ | 1-F, 3-Cl | n-propenyl | H |
| 415 | CH₂ | 1-Cl, 3-Br | n-propenyl | H |
| 416 | CH₂ | 1-Br, 3-Cl | n-propenyl | H |
| 417 | CH₂ | 1-Cl, 3-CF3 | n-propenyl | H |
| 418 | CH₂ | 11-CF₃, 3-Cl | n-propenyl | H |
| 419 | CH₂ | 1-Cl, 3-OCH₃ | n-propenyl | H |
| 420 | CH₂ | 1-OCH₃, 3-Cl | n-propenyl | H |
| 421 | CH₂ | 1-F, 3-Br | n-propenyl | H |
| 422 | CH₂ | 1-Br, 3-F | n-propenyl | H |
| 423 | CH₂ | 1-F, 3-CF₃ | n-propenyl | H |
| 424 | CH₂ | 1-CF₃, 3-F | n-propenyl | H |
| 425 | CH₂ | 1-F, 3-OCH₃ | n-propenyl | H |
| 426 | CH₂ | 1-OCH₃, 3-F | n-propenyl | H |
| 427 | CH₂ | 1-Br, 3-CF3 | n-propenyl | H |
| 428 | CH₂ | 1-CF₃, 3-Br | n-propenyl | H |
| 429 | CH₂ | 1-Br, 3-OCH₃ | n-propenyl | H |
| 430 | CH₂ | 1-OCH₃, 3-Br | n-propenyl | H |
| 431 | CH₂ | 1-CF₃, 3-OCH₃ | n-propenyl | H |
| 432 | CH₂ | 1-OCH₃, 3-CF₃ | n-propenyl | H |
| 433 | — | 1-CH₃ | benzyl | H |
| 434 | — | 2-CH₃ | benzyl | H |
| 435 | — | 3-CH₃ | benzyl | H |
| 436 | — | 4-CH₃ | benzyl | H |
| 437 | — | 1-CH₃, 3-CH₃ | benzyl | H |
| 438 | — | 1-CH₃, 3-CH₃, 4-CH₃ | benzyl | H |
| 439 | — | 3-CH₃, 4-CH₃ | benzyl | H |
| 440 | — | 1-Cl | benzyl | H |
| 441 | — | 2-Cl | benzyl | H |
| 442 | — | 3-Cl | benzyl | H |
| 443 | — | 4-Cl | benzyl | H |
| 444 | — | 1-Cl, 3-Cl | benzyl | H |
| 445 | — | 1-Cl, 3-Cl, 4-Cl | benzyl | H |
| 446 | — | 3-Cl, 4-Cl | benzyl | H |
| 447 | — | 1-F | benzyl | H |
| 448 | — | 2-F | benzyl | H |
| 449 | — | 3-F | benzyl | H |
| 450 | — | 4-F | benzyl | H |
| 451 | — | 1-F, 3-F | benzyl | H |
| 452 | — | 1-F, 3-F, 4-F | benzyl | H |
| 453 | — | 3-F, 4-F | benzyl | H |
| 454 | — | 1-Br | benzyl | H |
| 455 | — | 2-Br | benzyl | H |
| 456 | — | 3-Br | benzyl | H |
| 457 | — | 4-Br | benzyl | H |
| 458 | — | 1-Br, 3-Br | benzyl | H |
| 459 | — | 1-Br, 3-Br, 4-Br | benzyl | H |
| 460 | — | 3-Br, 4-Br | benzyl | H |
| 461 | — | 1-CF₃ | benzyl | H |
| 462 | — | 2-CF₃ | benzyl | H |
| 463 | — | 3-CF₃ | benzyl | H |
| 464 | — | 4-CF₃ | benzyl | H |
| 465 | — | 1CF₃, 3-CF₃ | benzyl | H |
| 466 | — | 1-CF₃, 3-CF₃, 4-CF₃ | benzyl | H |
| 467 | — | 3-CF₃, 4-CF3 | benzyl | H |
| 468 | — | 1-OCH₃ | benzyl | H |
| 469 | — | 2-OCH₃ | benzyl | H |
| 470 | — | 3-OCH₃ | benzyl | H |
| 471 | — | 4-OCH₃ | benzyl | H |
| 472 | — | 1-OCH₃, 3-OCH₃ | benzyl | H |
| 473 | — | 1-OCH₃, 3-OCH₃, 4-OCH₃ | benzyl | H |
| 474 | — | 3,4-OCH₃, OCH₃ | benzyl | H |
| 475 | — | 1-CH₃, 3-Cl | benzyl | H |
| 476 | — | 1-Cl, 3-CH₃ | benzyl | H |
| 477 | — | 1-CH₃, 3-F | benzyl | H |
| 478 | — | 1-F, 3-CH₃ | benzyl | H |
| 479 | — | 1-CH₃, 3-Br | benzyl | H |
| 480 | — | 1-Br, 3-CH₃ | benzyl | H |
| 481 | — | 1-CH₃, 3-CF₃ | benzyl | H |
| 482 | — | 1-CF₃, 3-CH₃ | benzyl | H |
| 483 | — | 1-CH₃, 3OCH₃ | benzyl | H |
| 484 | — | 1-OCH₃, 3-CH₃ | benzyl | H |
| 485 | — | 1-Cl, 3-F | benzyl | H |
| 486 | — | 1-F, 3-Cl | benzyl | H |
| 487 | — | 1-Cl, 3-Br | benzyl | H |
| 488 | — | 1-Br, 3-Cl | benzyl | H |
| 489 | — | 1-Cl, 3-CF3 | benzyl | H |
| 490 | — | 11-CF₃, 3-Cl | benzyl | H |
| 491 | — | 1-Cl, 3-OCH₃ | benzyl | H |
| 492 | — | 1-OCH₃, 3-Cl | benzyl | H |
| 493 | — | 1-F, 3-Br | benzyl | H |
| 494 | — | 1-Br, 3-F | benzyl | H |
| 495 | — | 1-F, 3-CF₃ | benzyl | H |
| 496 | — | 1-CF₃, 3-F | benzyl | H |
| 497 | — | 1-F, 3-OCH₃ | benzyl | H |
| 498 | — | 1-OCH₃, 3-F | benzyl | H |
| 499 | — | 1-Br, 3-CF3 | benzyl | H |
| 500 | — | 1-CF₃, 3-Br | benzyl | H |
| 501 | — | 1-Br, 3-OCH₃ | benzyl | H |
| 502 | — | 1-OCH₃, 3-Br | benzyl | H |
| 503 | — | 1-CF₃, 3-OCH₃ | benzyl | H |
| 504 | — | 1-OCH₃, 3-CF₃ | benzyl | H |
| 505 | CH₂ | 1-CH₃ | benzyl | H |
| 506 | CH₂ | 2-CH₃ | benzyl | H |
| 507 | CH₂ | 3-CH₃ | benzyl | H |
| 508 | CH₂ | 4-CH₃ | benzyl | H |
| 509 | CH₂ | 1-CH₃, 3-CH₃ | benzyl | H |
| 510 | CH₂ | 1-CH₃, 3-CH₃, 4-CH₃ | benzyl | H |
| 511 | CH₂ | 3-CH₃, 4-CH₃ | benzyl | H |
| 512 | CH₂ | 1-Cl | benzyl | H |
| 513 | CH₂ | 2-Cl | benzyl | H |
| 514 | CH₂ | 3-Cl | benzyl | H |
| 515 | CH₂ | 4-Cl | benzyl | H |
| 516 | CH₂ | 1-Cl, 3-Cl | benzyl | H |
| 517 | CH₂ | 1-Cl, 3-Cl, 4-Cl | benzyl | H |
| 518 | CH₂ | 3-Cl, 4-Cl | benzyl | H |
| 519 | CH₂ | 1-F | benzyl | H |
| 520 | CH₂ | 2-F | benzyl | H |
| 521 | CH₂ | 3-F | benzyl | H |
| 522 | CH₂ | 4-F | benzyl | H |
| 523 | CH₂ | 1-F, 3-F | benzyl | H |
| 524 | CH₂ | 1-F, 3-F, 4-F | benzyl | H |
| 525 | CH₂ | 3-F, 4-F | benzyl | H |
| 526 | CH₂ | 1-Br | benzyl | H |
| 527 | CH₂ | 2-Br | benzyl | H |
| 528 | CH₂ | 3-Br | benzyl | H |
| 529 | CH₂ | 4-Br | benzyl | H |
| 530 | CH₂ | 1-Br, 3-Br | benzyl | H |
| 531 | CH₂ | 1-Br, 3-Br, 4-Br | benzyl | H |
| 532 | CH₂ | 3-Br, 4-Br | benzyl | H |
| 533 | CH₂ | 1-CF₃ | benzyl | H |
| 534 | CH₂ | 2-CF₃ | benzyl | H |
| 535 | CH₂ | 3-CF₃ | benzyl | H |
| 536 | CH₂ | 4-CF₃ | benzyl | H |
| 537 | CH₂ | 1CF₃, 3-CF₃ | benzyl | H |
| 538 | CH₂ | 1-CF₃, 3-CF₃, 4-CF₃ | benzyl | H |
| 539 | CH₂ | 3-CF₃, 4-CF3 | benzyl | H |
| 540 | CH₂ | 1-OCH₃ | benzyl | H |
| 541 | CH₂ | 2-OCH₃ | benzyl | H |
| 542 | CH₂ | 3-OCH₃ | benzyl | H |
| 543 | CH₂ | 4-OCH₃ | benzyl | H |
| 544 | CH₂ | 1-OCH₃, 3-OCH₃ | benzyl | H |
| 545 | CH₂ | 1-OCH₃, 3-OCH₃, 4-OCH₃ | benzyl | H |
| 546 | CH₂ | 3,4-OCH₃, OCH₃ | benzyl | H |
| 547 | CH₂ | 1-CH₃, 3-Cl | benzyl | H |
| 548 | CH₂ | 1-Cl, 3-CH₃ | benzyl | H |
| 549 | CH₂ | 1-CH₃, 3-F | benzyl | H |
| 550 | CH₂ | 1-F, 3-CH₃ | benzyl | H |
| 551 | CH₂ | 1-CH₃, 3-Br | benzyl | H |
| 552 | CH₂ | 1-Br, 3-CH₃ | benzyl | H |
| 553 | CH₂ | 1-CH₃, 3-CF₃ | benzyl | H |
| 554 | CH₂ | 1-CF₃, 3-CH₃ | benzyl | H |
| 555 | CH₂ | 1-CH₃, 3OCH₃ | benzyl | H |
| 556 | CH₂ | 1-OCH₃, 3-CH₃ | benzyl | H |
| 557 | CH₂ | 1-Cl, 3-F | benzyl | H |
| 558 | CH₂ | 1-F, 3-Cl | benzyl | H |
| 559 | CH₂ | 1-Cl, 3-Br | benzyl | H |
| 560 | CH₂ | 1-Br, 3-Cl | benzyl | H |
| 561 | CH₂ | 1-Cl, 3-CF3 | benzyl | H |
| 562 | CH₂ | 11-CF₃, 3-Cl | benzyl | H |
| 563 | CH₂ | 1-Cl, 3-OCH₃ | benzyl | H |
| 564 | CH₂ | 1-OCH₃, 3-Cl | benzyl | H |
| 565 | CH₂ | 1-F, 3-Br | benzyl | H |
| 566 | CH₂ | 1-Br, 3-F | benzyl | H |
| 567 | CH₂ | 1-F, 3-CF₃ | benzyl | H |
| 568 | CH₂ | 1-CF₃, 3-F | benzyl | H |
| 569 | CH₂ | 1-F, 3-OCH₃ | benzyl | H |
| 570 | CH₂ | 1-OCH₃, 3-F | benzyl | H |
| 571 | CH₂ | 1-Br, 3-CF3 | benzyl | H |
| 572 | CH₂ | 1-CF₃, 3-Br | benzyl | H |
| 573 | CH₂ | 1-Br, 3-OCH₃ | benzyl | H |
| 574 | CH₂ | 1-OCH₃, 3-Br | benzyl | H |
| 575 | CH₂ | 1-CF₃, 3-OCH₃ | benzyl | H |
| 576 | CH₂ | 1-OCH₃, 3-CF₃ | benzyl | H |
| 577 | — | 1-CH₃ | F | H |
| 578 | — | 2-CH₃ | F | H |
| 579 | — | 3-CH₃ | F | H |
| 580 | — | 4-CH₃ | F | H |
| 581 | — | 1-CH₃, 3-CH₃ | F | H |
| 582 | — | 1-CH₃, 3-CH₃, 4-CH₃ | F | H |
| 583 | — | 3-CH₃, 4-CH₃ | F | H |
| 584 | — | 1-Cl | F | H |
| 585 | — | 2-Cl | F | H |
| 586 | — | 3-Cl | F | H |
| 587 | — | 4-Cl | F | H |
| 588 | — | 1-Cl, 3-Cl | F | H |
| 589 | — | 1-Cl, 3-Cl, 4-Cl | F | H |
| 590 | — | 3-Cl, 4-Cl | F | H |
| 591 | — | 1-F | F | H |
| 592 | — | 2-F | F | H |
| 593 | — | 3-F | F | H |
| 594 | — | 4-F | F | H |
| 595 | — | 1-F, 3-F | F | H |
| 596 | — | 1-F, 3-F, 4-F | F | H |
| 597 | — | 3-F, 4-F | F | H |
| 598 | — | 1-Br | F | H |
| 599 | — | 2-Br | F | H |
| 600 | — | 3-Br | F | H |
| 601 | — | 4-Br | F | H |
| 602 | — | 1-Br, 3-Br | F | H |
| 603 | — | 1-Br, 3-Br, 4-Br | F | H |
| 604 | — | 3-Br, 4-Br | F | H |
| 605 | — | 1-CF₃ | F | H |
| 606 | — | 2-CF₃ | F | H |
| 607 | — | 3-CF₃ | F | H |
| 608 | — | 4-CF₃ | F | H |
| 609 | — | 1CF₃, 3-CF₃ | F | H |
| 610 | — | 1-CF₃, 3-CF₃, 4-CF₃ | F | H |
| 611 | — | 3-CF₃, 4-CF3 | F | H |
| 612 | — | 1-OCH₃ | F | H |
| 613 | — | 2-OCH₃ | F | H |
| 614 | — | 3-OCH₃ | F | H |
| 615 | — | 4-OCH₃ | F | H |
| 616 | — | 1-OCH₃, 3-OCH₃ | F | H |
| 617 | — | 1-OCH₃, 3-OCH₃, 4-OCH₃ | F | H |
| 618 | — | 3,4-OCH₃, OCH₃ | F | H |
| 619 | — | 1-CH₃, 3-Cl | F | H |
| 620 | — | 1-Cl, 3-CH₃ | F | H |
| 621 | — | 1-CH₃, 3-F | F | H |
| 622 | — | 1-F, 3-CH₃ | F | H |
| 623 | — | 1-CH₃, 3-Br | F | H |
| 624 | — | 1-Br, 3-CH₃ | F | H |
| 625 | — | 1-CH₃, 3-CF₃ | F | H |
| 626 | — | 1-CF₃, 3-CH₃ | F | H |
| 627 | — | 1-CH₃, 3OCH₃ | F | H |
| 628 | — | 1-OCH₃, 3-CH₃ | F | H |
| 629 | — | 1-Cl, 3-F | F | H |
| 630 | — | 1-F, 3-Cl | F | H |
| 631 | — | 1-Cl, 3-Br | F | H |
| 632 | — | 1-Br, 3-Cl | F | H |
| 633 | — | 1-Cl, 3-CF₃ | F | H |
| 634 | — | 11-CF₃, 3-Cl | F | H |
| 635 | — | 1-Cl, 3-OCH₃ | F | H |
| 636 | — | 1-OCH₃, 3-Cl | F | H |
| 637 | — | 1-F, 3-Br | F | H |
| 638 | — | 1-Br, 3-F | F | H |
| 639 | — | 1-F, 3-CF₃ | F | H |
| 640 | — | 1-CF₃, 3-F | F | H |
| 641 | — | 1-F, 3-OCH₃ | F | H |
| 642 | — | 1-OCH₃, 3-F | F | H |
| 643 | — | 1-Br, 3-CF₃ | F | H |
| 644 | — | 1-CF₃, 3-Br | F | H |
| 645 | — | 1-Br, 3-OCH₃ | F | H |
| 646 | — | 1-OCH₃, 3-Br | F | H |
| 647 | — | 1-CF₃, 3-OCH₃ | F | H |
| 648 | — | 1-OCH₃, 3-CF₃ | F | H |
| 649 | CH₂ | 1-CH₃ | F | H |
| 650 | CH₂ | 2-CH₃ | F | H |
| 651 | CH₂ | 3-CH₃ | F | H |
| 652 | CH₂ | 4-CH₃ | F | H |
| 653 | CH₂ | 1-CH₃, 3-CH₃ | F | H |
| 654 | CH₂ | 1-CH₃, 3-CH₃, 4-CH₃ | F | H |
| 655 | CH₂ | 3-CH₃, 4-CH₃ | F | H |
| 656 | CH₂ | 1-Cl | F | H |
| 657 | CH₂ | 2-Cl | F | H |
| 658 | CH₂ | 3-Cl | F | H |
| 659 | CH₂ | 4-Cl | F | H |
| 660 | CH₂ | 1-Cl, 3-Cl | F | H |
| 661 | CH₂ | 1-Cl, 3-Cl, 4-Cl | F | H |
| 662 | CH₂ | 3-Cl, 4-Cl | F | H |
| 663 | CH₂ | 1-F | F | H |
| 664 | CH₂ | 2-F | F | H |
| 665 | CH₂ | 3-F | F | H |
| 666 | CH₂ | 4-F | F | H |
| 667 | CH₂ | 1-F, 3-F | F | H |
| 668 | CH₂ | 1-F, 3-F, 4-F | F | H |
| 669 | CH₂ | 3-F, 4-F | F | H |
| 670 | CH₂ | 1-Br | F | H |
| 671 | CH₂ | 2-Br | F | H |
| 672 | CH₂ | 3-Br | F | H |
| 673 | CH₂ | 4-Br | F | H |
| 674 | CH₂ | 1-Br, 3-Br | F | H |
| 675 | CH₂ | 1-Br, 3-Br, 4-Br | F | H |
| 676 | CH₂ | 3-Br, 4-Br | F | H |
| 677 | CH₂ | 1-CF₃ | F | H |
| 678 | CH₂ | 2-CF₃ | F | H |
| 679 | CH₂ | 3-CF₃ | F | H |
| 680 | CH₂ | 4-CF₃ | F | H |
| 681 | CH₂ | 1CF₃, 3-CF₃ | F | H |
| 682 | CH₂ | 1-CF₃, 3-CF₃, 4-CF₃ | F | H |
| 683 | CH₂ | 3-CF₃, 4-CF3 | F | H |
| 684 | CH₂ | 1-OCH₃ | F | H |
| 685 | CH₂ | 2-OCH₃ | F | H |
| 686 | CH₂ | 3-OCH₃ | F | H |
| 687 | CH₂ | 4-OCH₃ | F | H |
| 688 | CH₂ | 1-OCH₃, 3-OCH₃ | F | H |
| 689 | CH₂ | 1-OCH₃, 3-OCH₃, 4-OCH₃ | F | H |
| 690 | CH₂ | 3,4-OCH₃, OCH₃ | F | H |
| 691 | CH₂ | 1-CH₃, 3-Cl | F | H |
| 692 | CH₂ | 1-Cl, 3-CH₃ | F | H |
| 693 | CH₂ | 1-CH₃, 3-F | F | H |
| 694 | CH₂ | 1-F, 3-CH₃ | F | H |
| 695 | CH₂ | 1-CH₃, 3-Br | F | H |
| 696 | CH₂ | 1-Br, 3-CH₃ | F | H |
| 697 | CH₂ | 1-CH₃, 3-CF₃ | F | H |
| 698 | CH₂ | 1-CF₃, 3-CH₃ | F | H |
| 699 | CH₂ | 1-CH₃, 3OCH₃ | F | H |
| 700 | CH₂ | 1-OCH₃, 3-CH₃ | F | H |
| 701 | CH₂ | 1-Cl, 3-F | F | H |
| 702 | CH₂ | 1-F, 3-Cl | F | H |
| 703 | CH₂ | 1-Cl, 3-Br | F | H |
| 704 | CH₂ | 1-Br, 3-Cl | F | H |
| 705 | CH₂ | 1-Cl, 3-CF₃ | F | H |
| 706 | CH₂ | 11-CF₃, 3-Cl | F | H |
| 707 | CH₂ | 1-Cl, 3-OCH₃ | F | H |
| 708 | CH₂ | 1-OCH₃, 3-Cl | F | H |
| 709 | CH₂ | 1-F, 3-Br | F | H |
| 710 | CH₂ | 1-Br, 3-F | F | H |
| 711 | CH₂ | 1-F, 3-CF₃ | F | H |
| 712 | CH₂ | 1-CF₃, 3-F | F | H |
| 713 | CH₂ | 1-F, 3-OCH₃ | F | H |
| 714 | CH₂ | 1-OCH₃, 3-F | F | H |
| 715 | CH₂ | 1-Br, 3-CF₃ | F | H |
| 716 | CH₂ | 1-CF₃, 3-Br | F | H |
| 717 | CH₂ | 1-Br, 3-OCH₃ | F | H |
| 718 | CH₂ | 1-OCH₃, 3-Br | F | H |
| 719 | CH₂ | 1-CF₃, 3-OCH₃ | F | H |
| 720 | CH₂ | 1-OCH₃, 3-CF₃ | F | H |
| 721 | — | 1-CH₃ | CH₃ | CH₃ |
| 722 | — | 2-CH₃ | CH₃ | CH₃ |
| 723 | — | 3-CH₃ | CH₃ | CH₃ |
| 724 | — | 4-CH₃ | CH₃ | CH₃ |
| 725 | — | 1-CH₃, 3-CH₃ | CH₃ | CH₃ |
| 726 | — | 1-CH₃, 3-CH₃, 4-CH₃ | CH₃ | CH₃ |
| 727 | — | 3-CH₃, 4-CH₃ | CH₃ | CH₃ |
| 728 | — | 1-Cl | CH₃ | CH₃ |
| 729 | — | 2-Cl | CH₃ | CH₃ |
| 730 | — | 3-Cl | CH₃ | CH₃ |
| 731 | — | 4-Cl | CH₃ | CH₃ |
| 732 | — | 1-Cl, 3-Cl | CH₃ | CH₃ |
| 733 | — | 1-Cl, 3-Cl, 4-Cl | CH₃ | CH₃ |
| 734 | — | 3-Cl, 4-Cl | CH₃ | CH₃ |
| 735 | — | 1-F | CH₃ | CH₃ |
| 736 | — | 2-F | CH₃ | CH₃ |
| 737 | — | 3-F | CH₃ | CH₃ |
| 738 | — | 4-F | CH₃ | CH₃ |
| 739 | — | 1-F, 3-F | CH₃ | CH₃ |
| 740 | — | 1-F, 3-F, 4-F | CH₃ | CH₃ |
| 741 | — | 3-F, 4-F | CH₃ | CH₃ |
| 742 | — | 1-Br | CH₃ | CH₃ |
| 743 | — | 2-Br | CH₃ | CH₃ |
| 744 | — | 3-Br | CH₃ | CH₃ |
| 745 | — | 4-Br | CH₃ | CH₃ |
| 746 | — | 1-Br, 3-Br | CH₃ | CH₃ |
| 747 | — | 1-Br, 3-Br, 4-Br | CH₃ | CH₃ |
| 748 | — | 3-Br, 4-Br | CH₃ | CH₃ |
| 749 | — | 1-CF₃ | CH₃ | CH₃ |
| 750 | — | 2-CF₃ | CH₃ | CH₃ |
| 751 | — | 3-CF₃ | CH₃ | CH₃ |
| 752 | — | 4-CF₃ | CH₃ | CH₃ |
| 753 | — | 1CF₃, 3-CF₃ | CH₃ | CH₃ |
| 754 | — | 1-CF₃, 3-CF₃, 4-CF₃ | CH₃ | CH₃ |
| 755 | — | 3-CF₃, 4-CF3 | CH₃ | CH₃ |
| 756 | — | 1-OCH₃ | CH₃ | CH₃ |
| 757 | — | 2-OCH₃ | CH₃ | CH₃ |
| 758 | — | 3-OCH₃ | CH₃ | CH₃ |
| 759 | — | 4-OCH₃ | CH₃ | CH₃ |
| 760 | — | 1-OCH₃, 3-OCH₃ | CH₃ | CH₃ |
| 761 | — | 1-OCH₃, 3-OCH₃, 4-OCH₃ | CH₃ | CH₃ |
| 762 | — | 3,4-OCH₃, OCH₃ | CH₃ | CH₃ |
| 763 | — | 1-CH₃, 3-Cl | CH₃ | CH₃ |
| 764 | — | 1-Cl, 3-CH₃ | CH₃ | CH₃ |
| 765 | — | 1-CH₃, 3-F | CH₃ | CH₃ |
| 766 | — | 1-F, 3-CH₃ | CH₃ | CH₃ |
| 767 | — | 1-CH₃, 3-Br | CH₃ | CH₃ |
| 768 | — | 1-Br, 3-CH₃ | CH₃ | CH₃ |
| 769 | — | 1-CH₃, 3-CF₃ | CH₃ | CH₃ |
| 770 | — | 1-CF₃, 3-CH₃ | CH₃ | CH₃ |
| 771 | — | 1-CH₃, 3OCH₃ | CH₃ | CH₃ |
| 772 | — | 1-OCH₃, 3-CH₃ | CH₃ | CH₃ |
| 773 | — | 1-Cl, 3-F | CH₃ | CH₃ |
| 774 | — | 1-F, 3-Cl | CH₃ | CH₃ |
| 775 | — | 1-Cl, 3-Br | CH₃ | CH₃ |
| 776 | — | 1-Br, 3-Cl | CH₃ | CH₃ |
| 777 | — | 1-Cl, 3-CF3 | CH₃ | CH₃ |
| 778 | — | 11-CF₃, 3-Cl | CH₃ | CH₃ |
| 779 | — | 1-Cl, 3-OCH₃ | CH₃ | CH₃ |
| 780 | — | 1-OCH₃, 3-Cl | CH₃ | CH₃ |
| 781 | — | 1-F, 3-Br | CH₃ | CH₃ |
| 782 | — | 1-Br, 3-F | CH₃ | CH₃ |
| 783 | — | 1-F, 3-CF₃ | CH₃ | CH₃ |
| 784 | — | 1-CF₃, 3-F | CH₃ | CH₃ |
| 785 | — | 1-F, 3-OCH₃ | CH₃ | CH₃ |
| 786 | — | 1-OCH₃, 3-F | CH₃ | CH₃ |
| 787 | — | 1-Br, 3-CF3 | CH₃ | CH₃ |
| 788 | — | 1-CF₃, 3-Br | CH₃ | CH₃ |
| 789 | — | 1-Br, 3-OCH₃ | CH₃ | CH₃ |
| 790 | — | 1-OCH₃, 3-Br | CH₃ | CH₃ |
| 791 | — | 1-CF₃, 3-OCH₃ | CH₃ | CH₃ |
| 792 | — | 1-OCH₃, 3-CF₃ | CH₃ | CH₃ |
| 793 | CH₂ | 1-CH₃ | CH₃ | CH₃ |
| 794 | CH₂ | 2-CH₃ | CH₃ | CH₃ |
| 795 | CH₂ | 3-CH₃ | CH₃ | CH₃ |
| 796 | CH₂ | 4-CH₃ | CH₃ | CH₃ |
| 797 | CH₂ | 1-CH₃, 3-CH₃ | CH₃ | CH₃ |
| 798 | CH₂ | 1-CH₃, 3-CH₃, 4-CH₃ | CH₃ | CH₃ |
| 799 | CH₂ | 3-CH₃, 4-CH₃ | CH₃ | CH₃ |
| 800 | CH₂ | 1-Cl | CH₃ | CH₃ |
| 801 | CH₂ | 2-Cl | CH₃ | CH₃ |
| 802 | CH₂ | 3-Cl | CH₃ | CH₃ |
| 803 | CH₂ | 4-Cl | CH₃ | CH₃ |
| 804 | CH₂ | 1-Cl, 3-Cl | CH₃ | CH₃ |
| 805 | CH₂ | 1-Cl, 3-Cl, 4-Cl | CH₃ | CH₃ |
| 806 | CH₂ | 3-Cl, 4-Cl | CH₃ | CH₃ |
| 807 | CH₂ | 1-F | CH₃ | CH₃ |
| 808 | CH₂ | 2-F | CH₃ | CH₃ |
| 809 | CH₂ | 3-F | CH₃ | CH₃ |
| 810 | CH₂ | 4-F | CH₃ | CH₃ |
| 811 | CH₂ | 1-F, 3-F | CH₃ | CH₃ |
| 812 | CH₂ | 1-F, 3-F, 4-F | CH₃ | CH₃ |
| 813 | CH₂ | 3-F, 4-F | CH₃ | CH₃ |
| 814 | CH₂ | 1-Br | CH₃ | CH₃ |
| 815 | CH₂ | 2-Br | CH₃ | CH₃ |
| 816 | CH₂ | 3-Br | CH₃ | CH₃ |
| 817 | CH₂ | 4-Br | CH₃ | CH₃ |
| 818 | CH₂ | 1-Br, 3-Br | CH₃ | CH₃ |
| 819 | CH₂ | 1-Br, 3-Br, 4-Br | CH₃ | CH₃ |
| 820 | CH₂ | 3-Br, 4-Br | CH₃ | CH₃ |
| 821 | CH₂ | 1-CF₃ | CH₃ | CH₃ |
| 822 | CH₂ | 2-CF₃ | CH₃ | CH₃ |
| 823 | CH₂ | 3-CF₃ | CH₃ | CH₃ |
| 824 | CH₂ | 4-CF₃ | CH₃ | CH₃ |
| 825 | CH₂ | 1CF₃, 3-CF₃ | CH₃ | CH₃ |
| 826 | CH₂ | 1-CF₃, 3-CF₃, 4-CF₃ | CH₃ | CH₃ |
| 827 | CH₂ | 3-CF₃, 4-CF3 | CH₃ | CH₃ |
| 828 | CH₂ | 1-OCH₃ | CH₃ | CH₃ |
| 829 | CH₂ | 2-OCH₃ | CH₃ | CH₃ |
| 830 | CH₂ | 3-OCH₃ | CH₃ | CH₃ |
| 831 | CH₂ | 4-OCH₃ | CH₃ | CH₃ |
| 832 | CH₂ | 1-OCH₃, 3-OCH₃ | CH₃ | CH₃ |
| 833 | CH₂ | 1-OCH₃, 3-OCH₃, 4-OCH₃ | CH₃ | CH₃ |
| 834 | CH₂ | 3,4-OCH₃, OCH₃ | CH₃ | CH₃ |
| 835 | CH₂ | 1-CH₃, 3-Cl | CH₃ | CH₃ |
| 836 | CH₂ | 1-Cl, 3-CH₃ | CH₃ | CH₃ |
| 837 | CH₂ | 1-CH₃, 3-F | CH₃ | CH₃ |
| 838 | CH₂ | 1-F, 3-CH₃ | CH₃ | CH₃ |
| 839 | CH₂ | 1-CH₃, 3-Br | CH₃ | CH₃ |
| 840 | CH₂ | 1-Br, 3-CH₃ | CH₃ | CH₃ |
| 841 | CH₂ | 1-CH₃, 3-CF₃ | CH₃ | CH₃ |
| 842 | CH₂ | 1-CF₃, 3-CH₃ | CH₃ | CH₃ |
| 843 | CH₂ | 1-CH₃, 3OCH₃ | CH₃ | CH₃ |
| 844 | CH₂ | 1-OCH₃, 3-CH₃ | CH₃ | CH₃ |
| 845 | CH₂ | 1-Cl, 3-F | CH₃ | CH₃ |
| 846 | CH₂ | 1-F, 3-Cl | CH₃ | CH₃ |
| 847 | CH₂ | 1-Cl, 3-Br | CH₃ | CH₃ |
| 848 | CH₂ | 1-Br, 3-Cl | CH₃ | CH₃ |
| 849 | CH₂ | 1-Cl, 3-CF3 | CH₃ | CH₃ |
| 850 | CH₂ | 11-CF₃, 3-Cl | CH₃ | CH₃ |
| 851 | CH₂ | 1-Cl, 3-OCH₃ | CH₃ | CH₃ |
| 852 | CH₂ | 1-OCH₃, 3-Cl | CH₃ | CH₃ |
| 853 | CH₂ | 1-F, 3-Br | CH₃ | CH₃ |
| 854 | CH₂ | 1-Br, 3-F | CH₃ | CH₃ |
| 855 | CH₂ | 1-F, 3-CF₃ | CH₃ | CH₃ |
| 856 | CH₂ | 1-CF₃, 3-F | CH₃ | CH₃ |
| 857 | CH₂ | 1-F, 3-OCH₃ | CH₃ | CH₃ |
| 858 | CH₂ | 1-OCH₃, 3-F | CH₃ | CH₃ |
| 859 | CH₂ | 1-Br, 3-CF3 | CH₃ | CH₃ |
| 860 | CH₂ | 1-CF₃, 3-Br | CH₃ | CH₃ |
| 861 | CH₂ | 1-Br, 3-OCH₃ | CH₃ | CH₃ |
| 862 | CH₂ | 1-OCH₃, 3-Br | CH₃ | CH₃ |
| 863 | CH₂ | 1-CF₃, 3-OCH₃ | CH₃ | CH₃ |
| 864 | CH₂ | 1-OCH₃, 3-CF₃ | CH₃ | CH₃ |
| 865 | — | 1-CH₃ | n-propenyl | CH₃ |
| 866 | — | 2-CH₃ | n-propenyl | CH₃ |
| 867 | — | 3-CH₃ | n-propenyl | CH₃ |
| 868 | — | 4-CH₃ | n-propenyl | CH₃ |
| 869 | — | 1-CH₃, 3-CH₃ | n-propenyl | CH₃ |
| 870 | — | 1-CH₃, 3-CH₃, 4-CH₃ | n-propenyl | CH₃ |
| 871 | — | 3-CH₃, 4-CH₃ | n-propenyl | CH₃ |
| 872 | — | 1-Cl | n-propenyl | CH₃ |
| 873 | — | 2-Cl | n-propenyl | CH₃ |
| 874 | — | 3-Cl | n-propenyl | CH₃ |
| 875 | — | 4-Cl | n-propenyl | CH₃ |
| 876 | — | 1-Cl, 3-Cl | n-propenyl | CH₃ |
| 877 | — | 1-Cl, 3-Cl, 4-Cl | n-propenyl | CH₃ |
| 878 | — | 3-Cl, 4-Cl | n-propenyl | CH₃ |
| 879 | — | 1-F | n-propenyl | CH₃ |
| 880 | — | 2-F | n-propenyl | CH₃ |
| 881 | — | 3-F | n-propenyl | CH₃ |
| 882 | — | 4-F | n-propenyl | CH₃ |
| 883 | — | 1-F, 3-F | n-propenyl | CH₃ |
| 884 | — | 1-F, 3-F, 4-F | n-propenyl | CH₃ |
| 885 | — | 3-F, 4-F | n-propenyl | CH₃ |
| 886 | — | 1-Br | n-propenyl | CH₃ |
| 887 | — | 2-Br | n-propenyl | CH₃ |
| 888 | — | 3-Br | n-propenyl | CH₃ |
| 889 | — | 4-Br | n-propenyl | CH₃ |
| 890 | — | 1-Br, 3-Br | n-propenyl | CH₃ |
| 891 | — | 1-Br, 3-Br, 4-Br | n-propenyl | CH₃ |
| 892 | — | 3-Br, 4-Br | n-propenyl | CH₃ |
| 893 | — | 1-CF₃ | n-propenyl | CH₃ |
| 894 | — | 2-CF₃ | n-propenyl | CH₃ |
| 895 | — | 3-CF₃ | n-propenyl | CH₃ |
| 896 | — | 4-CF₃ | n-propenyl | CH₃ |
| 897 | — | 1CF₃, 3-CF₃ | n-propenyl | CH₃ |
| 898 | — | 1-CF₃, 3-CF₃, 4-CF₃ | n-propenyl | CH₃ |
| 899 | — | 3-CF₃, 4-CF3 | n-propenyl | CH₃ |
| 900 | — | 1-OCH₃ | n-propenyl | CH₃ |
| 901 | — | 2-OCH₃ | n-propenyl | CH₃ |
| 902 | — | 3-OCH₃ | n-propenyl | CH₃ |
| 903 | — | 4-OCH₃ | n-propenyl | CH₃ |
| 904 | — | 1-OCH₃, 3-OCH₃ | n-propenyl | CH₃ |
| 905 | — | 1-OCH₃, 3-OCH₃, 4-OCH₃ | n-propenyl | CH₃ |
| 906 | — | 3,4-OCH₃, OCH₃ | n-propenyl | CH₃ |
| 907 | — | 1-CH₃, 3-Cl | n-propenyl | CH₃ |
| 908 | — | 1-Cl, 3-CH₃ | n-propenyl | CH₃ |
| 909 | — | 1-CH₃, 3-F | n-propenyl | CH₃ |
| 910 | — | 1-F, 3-CH₃ | n-propenyl | CH₃ |
| 911 | — | 1-CH₃, 3-Br | n-propenyl | CH₃ |
| 912 | — | 1-Br, 3-CH₃ | n-propenyl | CH₃ |
| 913 | — | 1-CH₃, 3-CF₃ | n-propenyl | CH₃ |
| 914 | — | 1-CF₃, 3-CH₃ | n-propenyl | CH₃ |
| 915 | — | 1-CH₃, 3OCH₃ | n-propenyl | CH₃ |
| 916 | — | 1-OCH₃, 3-CH₃ | n-propenyl | CH₃ |
| 917 | — | 1-Cl, 3-F | n-propenyl | CH₃ |
| 918 | — | 1-F, 3-Cl | n-propenyl | CH₃ |
| 919 | — | 1-Cl, 3-Br | n-propenyl | CH₃ |
| 920 | — | 1-Br, 3-Cl | n-propenyl | CH₃ |
| 921 | — | 1-Cl, 3-CF3 | n-propenyl | CH₃ |
| 922 | — | 11-CF₃, 3-Cl | n-propenyl | CH₃ |
| 923 | — | 1-Cl, 3-OCH₃ | n-propenyl | CH₃ |
| 924 | — | 1-OCH₃, 3-Cl | n-propenyl | CH₃ |
| 925 | — | 1-F, 3-Br | n-propenyl | CH₃ |
| 926 | — | 1-Br, 3-F | n-propenyl | CH₃ |
| 927 | — | 1-F, 3-CF₃ | n-propenyl | CH₃ |
| 928 | — | 1-CF₃, 3-F | n-propenyl | CH₃ |
| 929 | — | 1-F, 3-OCH₃ | n-propenyl | CH₃ |
| 930 | — | 1-OCH₃, 3-F | n-propenyl | CH₃ |
| 931 | — | 1-Br, 3-CF3 | n-propenyl | CH₃ |
| 932 | — | 1-CF₃, 3-Br | n-propenyl | CH₃ |
| 933 | — | 1-Br, 3-OCH₃ | n-propenyl | CH₃ |
| 934 | — | 1-OCH₃, 3-Br | n-propenyl | CH₃ |
| 935 | — | 1-CF₃, 3-OCH₃ | n-propenyl | CH₃ |
| 936 | — | 1-OCH₃, 3-CF₃ | n-propenyl | CH₃ |
| 937 | CH₂ | 1-CH₃ | n-propenyl | CH₃ |
| 938 | CH₂ | 2-CH₃ | n-propenyl | CH₃ |
| 939 | CH₂ | 3-CH₃ | n-propenyl | CH₃ |
| 940 | CH₂ | 4-CH₃ | n-propenyl | CH₃ |
| 941 | CH₂ | 1-CH₃, 3-CH₃ | n-propenyl | CH₃ |
| 942 | CH₂ | 1-CH₃, 3-CH₃, 4-CH₃ | n-propenyl | CH₃ |
| 943 | CH₂ | 3-CH₃, 4-CH₃ | n-propenyl | CH₃ |
| 944 | CH₂ | 1-Cl | n-propenyl | CH₃ |
| 945 | CH₂ | 2-Cl | n-propenyl | CH₃ |
| 946 | CH₂ | 3-Cl | n-propenyl | CH₃ |
| 947 | CH₂ | 4-Cl | n-propenyl | CH₃ |
| 948 | CH₂ | 1-Cl, 3-Cl | n-propenyl | CH₃ |
| 949 | CH₂ | 1-Cl, 3-Cl, 4-Cl | n-propenyl | CH₃ |
| 950 | CH₂ | 3-Cl, 4-Cl | n-propenyl | CH₃ |
| 951 | CH₂ | 1-F | n-propenyl | CH₃ |
| 952 | CH₂ | 2-F | n-propenyl | CH₃ |
| 953 | CH₂ | 3-F | n-propenyl | CH₃ |
| 954 | CH₂ | 4-F | n-propenyl | CH₃ |
| 955 | CH₂ | 1-F, 3-F | n-propenyl | CH₃ |
| 956 | CH₂ | 1-F, 3-F, 4-F | n-propenyl | CH₃ |
| 957 | CH₂ | 3-F, 4-F | n-propenyl | CH₃ |
| 958 | CH₂ | 1-Br | n-propenyl | CH₃ |
| 959 | CH₂ | 2-Br | n-propenyl | CH₃ |
| 960 | CH₂ | 3-Br | n-propenyl | CH₃ |
| 961 | CH₂ | 4-Br | n-propenyl | CH₃ |
| 962 | CH₂ | 1-Br, 3-Br | n-propenyl | CH₃ |
| 963 | CH₂ | 1-Br, 3-Br, 4-Br | n-propenyl | CH₃ |
| 964 | CH₂ | 3-Br, 4-Br | n-propenyl | CH₃ |
| 965 | CH₂ | 1-CF₃ | n-propenyl | CH₃ |
| 966 | CH₂ | 2-CF₃ | n-propenyl | CH₃ |
| 967 | CH₂ | 3-CF₃ | n-propenyl | CH₃ |
| 968 | CH₂ | 4-CF₃ | n-propenyl | CH₃ |
| 969 | CH₂ | 1CF₃, 3-CF₃ | n-propenyl | CH₃ |
| 970 | CH₂ | 1-CF₃, 3-CF₃, 4-CF₃ | n-propenyl | CH₃ |
| 971 | CH₂ | 3-CF₃, 4-CF3 | n-propenyl | CH₃ |
| 972 | CH₂ | 1-OCH₃ | n-propenyl | CH₃ |
| 973 | CH₂ | 2-OCH₃ | n-propenyl | CH₃ |
| 974 | CH₂ | 3-OCH₃ | n-propenyl | CH₃ |
| 975 | CH₂ | 4-OCH₃ | n-propenyl | CH₃ |
| 976 | CH₂ | 1-OCH₃, 3-OCH₃ | n-propenyl | CH₃ |
| 977 | CH₂ | 1-OCH₃, 3-OCH₃, 4-OCH₃ | n-propenyl | CH₃ |
| 978 | CH₂ | 3,4-OCH₃, OCH₃ | n-propenyl | CH₃ |
| 979 | CH₂ | 1-CH₃, 3-Cl | n-propenyl | CH₃ |
| 980 | CH₂ | 1-Cl, 3-CH₃ | n-propenyl | CH₃ |
| 981 | CH₂ | 1-CH₃, 3-F | n-propenyl | CH₃ |
| 982 | CH₂ | 1-F, 3-CH₃ | n-propenyl | CH₃ |
| 983 | CH₂ | 1-CH₃, 3-Br | n-propenyl | CH₃ |
| 984 | CH₂ | 1-Br, 3-CH₃ | n-propenyl | CH₃ |
| 985 | CH₂ | 1-CH₃, 3-CF₃ | n-propenyl | CH₃ |
| 986 | CH₂ | 1-CF₃, 3-CH₃ | n-propenyl | CH₃ |
| 987 | CH₂ | 1-CH₃, 3OCH₃ | n-propenyl | CH₃ |
| 988 | CH₂ | 1-OCH₃, 3-CH₃ | n-propenyl | CH₃ |
| 989 | CH₂ | 1-Cl, 3-F | n-propenyl | CH₃ |
| 990 | CH₂ | 1-F, 3-Cl | n-propenyl | CH₃ |
| 991 | CH₂ | 1-Cl, 3-Br | n-propenyl | CH₃ |
| 992 | CH₂ | 1-Br, 3-Cl | n-propenyl | CH₃ |
| 993 | CH₂ | 1-Cl, 3-CF3 | n-propenyl | CH₃ |
| 994 | CH₂ | 11-CF₃, 3-Cl | n-propenyl | CH₃ |
| 995 | CH₂ | 1-Cl, 3-OCH₃ | n-propenyl | CH₃ |
| 996 | CH₂ | 1-OCH₃, 3-Cl | n-propenyl | CH₃ |
| 997 | CH₂ | 1-F, 3-Br | n-propenyl | CH₃ |
| 998 | CH₂ | 1-Br, 3-F | n-propenyl | CH₃ |
| 999 | CH₂ | 1-F, 3-CF₃ | n-propenyl | CH₃ |
| 1000 | CH₂ | 1-CF₃, 3-F | n-propenyl | CH₃ |
| 1001 | CH₂ | 1-F, 3-OCH₃ | n-propenyl | CH₃ |
| 1002 | CH₂ | 1-OCH₃, 3-F | n-propenyl | CH₃ |
| 1003 | CH₂ | 1-Br, 3-CF3 | n-propenyl | CH₃ |
| 1004 | CH₂ | 1-CF₃, 3-Br | n-propenyl | CH₃ |
| 1005 | CH₂ | 1-Br, 3-OCH₃ | n-propenyl | CH₃ |
| 1006 | CH₂ | 1-OCH₃, 3-Br | n-propenyl | CH₃ |
| 1007 | CH₂ | 1-CF₃, 3-OCH₃ | n-propenyl | CH₃ |
| 1008 | CH₂ | 1-OCH₃, 3-CF₃ | n-propenyl | CH₃ |
| 1009 | — | 1-CH₃ | benzyl | CH₃ |
| 1010 | — | 2-CH₃ | benzyl | CH₃ |
| 1011 | — | 3-CH₃ | benzyl | CH₃ |
| 1012 | — | 4-CH₃ | benzyl | CH₃ |
| 1013 | — | 1-CH₃, 3-CH₃ | benzyl | CH₃ |
| 1014 | — | 1-CH₃, 3-CH₃, 4-CH₃ | benzyl | CH₃ |
| 1015 | — | 3-CH₃, 4-CH₃ | benzyl | CH₃ |
| 1016 | — | 1-Cl | benzyl | CH₃ |
| 1017 | — | 2-Cl | benzyl | CH₃ |
| 1018 | — | 3-Cl | benzyl | CH₃ |
| 1019 | — | 4-Cl | benzyl | CH₃ |
| 1020 | — | 1-Cl, 3-Cl | benzyl | CH₃ |
| 1021 | — | 1-Cl, 3-Cl, 4-Cl | benzyl | CH₃ |
| 1022 | — | 3-Cl, 4-Cl | benzyl | CH₃ |
| 1023 | — | 1-F | benzyl | CH₃ |
| 1024 | — | 2-F | benzyl | CH₃ |
| 1025 | — | 3-F | benzyl | CH₃ |
| 1026 | — | 4-F | benzyl | CH₃ |
| 1027 | — | 1-F, 3-F | benzyl | CH₃ |
| 1028 | — | 1-F, 3-F, 4-F | benzyl | CH₃ |
| 1029 | — | 3-F, 4-F | benzyl | CH₃ |
| 1030 | — | 1-Br | benzyl | CH₃ |
| 1031 | — | 2-Br | benzyl | CH₃ |
| 1032 | — | 3-Br | benzyl | CH₃ |
| 1033 | — | 4-Br | benzyl | CH₃ |
| 1034 | — | 1-Br, 3-Br | benzyl | CH₃ |
| 1035 | — | 1-Br, 3-Br, 4-Br | benzyl | CH₃ |
| 1036 | — | 3-Br, 4-Br | benzyl | CH₃ |
| 1037 | — | 1-CF₃ | benzyl | CH₃ |
| 1038 | — | 2-CF₃ | benzyl | CH₃ |
| 1039 | — | 3-CF₃ | benzyl | CH₃ |
| 1040 | — | 4-CF₃ | benzyl | CH₃ |
| 1041 | — | 1CF₃, 3-CF₃ | benzyl | CH₃ |
| 1042 | — | 1-CF₃, 3-CF₃, 4-CF₃ | benzyl | CH₃ |
| 1043 | — | 3-CF₃, 4-CF3 | benzyl | CH₃ |
| 1044 | — | 1-OCH₃ | benzyl | CH₃ |
| 1045 | — | 2-OCH₃ | benzyl | CH₃ |
| 1046 | — | 3-OCH₃ | benzyl | CH₃ |
| 1047 | — | 4-OCH₃ | benzyl | CH₃ |
| 1048 | — | 1-OCH₃, 3-OCH₃ | benzyl | CH₃ |
| 1049 | — | 1-OCH₃, 3-OCH₃, 4-OCH₃ | benzyl | CH₃ |
| 1050 | — | 3,4-OCH₃, OCH₃ | benzyl | CH₃ |
| 1051 | — | 1-CH₃, 3-Cl | benzyl | CH₃ |
| 1052 | — | 1-Cl, 3-CH₃ | benzyl | CH₃ |
| 1053 | — | 1-CH₃, 3-F | benzyl | CH₃ |
| 1054 | — | 1-F, 3-CH₃ | benzyl | CH₃ |
| 1055 | — | 1-CH₃, 3-Br | benzyl | CH₃ |
| 1056 | — | 1-Br, 3-CH₃ | benzyl | CH₃ |
| 1057 | — | 1-CH₃, 3-CF₃ | benzyl | CH₃ |
| 1058 | — | 1-CF₃, 3-CH₃ | benzyl | CH₃ |
| 1059 | — | 1-CH₃, 3OCH₃ | benzyl | CH₃ |
| 1060 | — | 1-OCH₃, 3-CH₃ | benzyl | CH₃ |
| 1061 | — | 1-Cl, 3-F | benzyl | CH₃ |
| 1062 | — | 1-F, 3-Cl | benzyl | CH₃ |
| 1063 | — | 1-Cl, 3-Br | benzyl | CH₃ |
| 1064 | — | 1-Br, 3-Cl | benzyl | CH₃ |
| 1065 | — | 1-Cl, 3-CF3 | benzyl | CH₃ |
| 1066 | — | 11-CF₃, 3-Cl | benzyl | CH₃ |
| 1067 | — | 1-Cl, 3-OCH₃ | benzyl | CH₃ |
| 1068 | — | 1-OCH₃, 3-Cl | benzyl | CH₃ |
| 1069 | — | 1-F, 3-Br | benzyl | CH₃ |
| 1070 | — | 1-Br, 3-F | benzyl | CH₃ |
| 1071 | — | 1-F, 3-CF₃ | benzyl | CH₃ |
| 1072 | — | 1-CF₃, 3-F | benzyl | CH₃ |
| 1073 | — | 1-F, 3-OCH₃ | benzyl | CH₃ |
| 1074 | — | 1-OCH₃, 3-F | benzyl | CH₃ |
| 1075 | — | 1-Br, 3-CF3 | benzyl | CH₃ |
| 1076 | — | 1-CF₃, 3-Br | benzyl | CH₃ |
| 1077 | — | 1-Br, 3-OCH₃ | benzyl | CH₃ |
| 1078 | — | 1-OCH₃, 3-Br | benzyl | CH₃ |
| 1079 | — | 1-CF₃, 3-OCH₃ | benzyl | CH₃ |
| 1080 | — | 1-OCH₃, 3-CF₃ | benzyl | CH₃ |
| 1081 | CH₂ | 1-CH₃ | benzyl | CH₃ |
| 1082 | CH₂ | 2-CH₃ | benzyl | CH₃ |
| 1083 | CH₂ | 3-CH₃ | benzyl | CH₃ |
| 1084 | CH₂ | 4-CH₃ | benzyl | CH₃ |
| 1085 | CH₂ | 1-CH₃, 3-CH₃ | benzyl | CH₃ |
| 1086 | CH₂ | 1-CH₃, 3-CH₃, 4-CH₃ | benzyl | CH₃ |
| 1087 | CH₂ | 3-CH₃, 4-CH₃ | benzyl | CH₃ |
| 1088 | CH₂ | 1-Cl | benzyl | CH₃ |
| 1089 | CH₂ | 2-Cl | benzyl | CH₃ |
| 1090 | CH₂ | 3-Cl | benzyl | CH₃ |
| 1091 | CH₂ | 4-Cl | benzyl | CH₃ |
| 1092 | CH₂ | 1-Cl, 3-Cl | benzyl | CH₃ |
| 1093 | CH₂ | 1-Cl, 3-Cl, 4-Cl | benzyl | CH₃ |
| 1094 | CH₂ | 3-Cl, 4-Cl | benzyl | CH₃ |
| 1095 | CH₂ | 1-F | benzyl | CH₃ |
| 1096 | CH₂ | 2-F | benzyl | CH₃ |
| 1097 | CH₂ | 3-F | benzyl | CH₃ |
| 1098 | CH₂ | 4-F | benzyl | CH₃ |
| 1099 | CH₂ | 1-F, 3-F | benzyl | CH₃ |
| 1100 | CH₂ | 1-F, 3-F, 4-F | benzyl | CH₃ |
| 1101 | CH₂ | 3-F, 4-F | benzyl | CH₃ |
| 1102 | CH₂ | 1-Br | benzyl | CH₃ |
| 1103 | CH₂ | 2-Br | benzyl | CH₃ |
| 1104 | CH₂ | 3-Br | benzyl | CH₃ |
| 1105 | CH₂ | 4-Br | benzyl | CH₃ |
| 1106 | CH₂ | 1-Br, 3-Br | benzyl | CH₃ |
| 1107 | CH₂ | 1-Br, 3-Br, 4-Br | benzyl | CH₃ |
| 1108 | CH₂ | 3-Br, 4-Br | benzyl | CH₃ |
| 1109 | CH₂ | 1-CF₃ | benzyl | CH₃ |
| 1110 | CH₂ | 2-CF₃ | benzyl | CH₃ |
| 1111 | CH₂ | 3-CF₃ | benzyl | CH₃ |
| 1112 | CH₂ | 4-CF₃ | benzyl | CH₃ |
| 1113 | CH₂ | 1CF₃, 3-CF₃ | benzyl | CH₃ |
| 1114 | CH₂ | 1-CF₃, 3-CF₃, 4-CF₃ | benzyl | CH₃ |
| 1115 | CH₂ | 3-CF₃, 4-CF₃ | benzyl | CH₃ |
| 1116 | CH₂ | 1-OCH₃ | benzyl | CH₃ |
| 1117 | CH₂ | 2-OCH₃ | benzyl | CH₃ |
| 1118 | CH₂ | 3-OCH₃ | benzyl | CH₃ |
| 1119 | CH₂ | 4-OCH₃ | benzyl | CH₃ |
| 1120 | CH₂ | 1-OCH₃, 3-OCH₃ | benzyl | CH₃ |
| 1121 | CH₂ | 1-OCH₃, 3-OCH₃, 4-OCH₃ | benzyl | CH₃ |
| 1122 | CH₂ | 3,4-OCH₃, OCH₃ | benzyl | CH₃ |
| 1123 | CH₂ | 1-CH₃, 3-Cl | benzyl | CH₃ |
| 1124 | CH₂ | 1-Cl, 3-CH₃ | benzyl | CH₃ |
| 1125 | CH₂ | 1-CH₃, 3-F | benzyl | CH₃ |
| 1126 | CH₂ | 1-F, 3-CH₃ | benzyl | CH₃ |
| 1127 | CH₂ | 1-CH₃, 3-Br | benzyl | CH₃ |
| 1128 | CH₂ | 1-Br, 3-CH₃ | benzyl | CH₃ |
| 1129 | CH₂ | 1-CH₃, 3-CF₃ | benzyl | CH₃ |
| 1130 | CH₂ | 1-CF₃, 3-CH₃ | benzyl | CH₃ |
| 1131 | CH₂ | 1-CH₃, 3OCH₃ | benzyl | CH₃ |
| 1132 | CH₂ | 1-OCH₃, 3-CH₃ | benzyl | CH₃ |
| 1133 | CH₂ | 1-Cl, 3-F | benzyl | CH₃ |
| 1134 | CH₂ | 1-F, 3-Cl | benzyl | CH₃ |
| 1135 | CH₂ | 1-Cl, 3-Br | benzyl | CH₃ |
| 1136 | CH₂ | 1-Br, 3-Cl | benzyl | CH₃ |
| 1137 | CH₂ | 1-Cl, 3-CF3 | benzyl | CH₃ |
| 1138 | CH₂ | 11-CF₃, 3-Cl | benzyl | CH₃ |
| 1139 | CH₂ | 1-Cl, 3-OCH₃ | benzyl | CH₃ |
| 1140 | CH₂ | 1-OCH₃, 3-Cl | benzyl | CH₃ |
| 1141 | CH₂ | 1-F, 3-Br | benzyl | CH₃ |
| 1142 | CH₂ | 1-Br, 3-F | benzyl | CH₃ |
| 1143 | CH₂ | 1-F, 3-CF₃ | benzyl | CH₃ |
| 1144 | CH₂ | 1-CF₃, 3-F | benzyl | CH₃ |
| 1145 | CH₂ | 1-F, 3-OCH₃ | benzyl | CH₃ |
| 1146 | CH₂ | 1-OCH₃, 3-F | benzyl | CH₃ |
| 1147 | CH₂ | 1-Br, 3-CF3 | benzyl | CH₃ |
| 1148 | CH₂ | 1-CF₃, 3-Br | benzyl | CH₃ |
| 1149 | CH₂ | 1-Br, 3-OCH₃ | benzyl | CH₃ |
| 1150 | CH₂ | 1-OCH₃, 3-Br | benzyl | CH₃ |
| 1151 | CH₂ | 1-CF₃, 3-OCH₃ | benzyl | CH₃ |
| 1152 | CH₂ | 1-OCH₃, 3-CF₃ | benzyl | CH₃ |
| 1153 | — | 1-CH₃ | F | F |
| 1154 | — | 2-CH₃ | F | F |
| 1155 | — | 3-CH₃ | F | F |
| 1156 | — | 4-CH₃ | F | F |
| 1157 | — | 1-CH₃, 3-CH₃ | F | F |
| 1158 | — | 1-CH₃, 3-CH₃, 4-CH₃ | F | F |
| 1159 | — | 3-CH₃, 4-CH₃ | F | F |
| 1160 | — | 1-Cl | F | F |
| 1161 | — | 2-Cl | F | F |
| 1162 | — | 3-Cl | F | F |
| 1163 | — | 4-Cl | F | F |
| 1164 | — | 1-Cl, 3-Cl | F | F |
| 1165 | — | 1-Cl, 3-Cl, 4-Cl | F | F |
| 1166 | — | 3-Cl, 4-Cl | F | F |
| 1167 | — | 1-F | F | F |
| 1168 | — | 2-F | F | F |
| 1169 | — | 3-F | F | F |
| 1170 | — | 4-F | F | F |
| 1171 | — | 1-F, 3-F | F | F |
| 1172 | — | 1-F, 3-F, 4-F | F | F |
| 1173 | — | 3-F, 4-F | F | F |
| 1174 | — | 1-Br | F | F |
| 1175 | — | 2-Br | F | F |
| 1176 | — | 3-Br | F | F |
| 1177 | — | 4-Br | F | F |
| 1178 | — | 1-Br, 3-Br | F | F |
| 1179 | — | 1-Br, 3-Br, 4-Br | F | F |
| 1180 | — | 3-Br, 4-Br | F | F |
| 1181 | — | 1-CF₃ | F | F |
| 1182 | — | 2-CF₃ | F | F |
| 1183 | — | 3-CF₃ | F | F |
| 1184 | — | 4-CF₃ | F | F |
| 1185 | — | 1CF₃, 3-CF₃ | F | F |
| 1186 | — | 1-CF₃, 3-CF₃, 4-CF₃ | F | F |
| 1187 | — | 3-CF₃, 4-CF3 | F | F |
| 1188 | — | 1-OCH₃ | F | F |
| 1189 | — | 2-OCH₃ | F | F |
| 1190 | — | 3-OCH₃ | F | F |
| 1191 | — | 4-OCH₃ | F | F |
| 1192 | — | 1-OCH₃, 3-OCH₃ | F | F |
| 1193 | — | 1-OCH₃, 3-OCH₃, 4-OCH₃ | F | F |
| 1194 | — | 3,4-OCH₃, OCH₃ | F | F |
| 1195 | — | 1-CH₃, 3-Cl | F | F |
| 1196 | — | 1-Cl, 3-CH₃ | F | F |
| 1197 | — | 1-CH₃, 3-F | F | F |
| 1198 | — | 1-F, 3-CH₃ | F | F |
| 1199 | — | 1-CH₃, 3-Br | F | F |
| 1200 | — | 1-Br, 3-CH₃ | F | F |
| 1201 | — | 1-CH₃, 3-CF₃ | F | F |
| 1202 | — | 1-CF₃, 3-CH₃ | F | F |
| 1203 | — | 1-CH₃, 3OCH₃ | F | F |
| 1204 | — | 1-OCH₃, 3-CH₃ | F | F |
| 1205 | — | 1-Cl, 3-F | F | F |
| 1206 | — | 1-F, 3-Cl | F | F |
| 1207 | — | 1-Cl, 3-Br | F | F |
| 1208 | — | 1-Br, 3-Cl | F | F |
| 1209 | — | 1-Cl, 3-CF₃ | F | F |
| 1210 | — | 11-CF₃, 3-Cl | F | F |
| 1211 | — | 1-Cl, 3-OCH₃ | F | F |
| 1212 | — | 1-OCH₃, 3-Cl | F | F |
| 1213 | — | 1-F, 3-Br | F | F |
| 1214 | — | 1-Br, 3-F | F | F |
| 1215 | — | 1-F, 3-CF₃ | F | F |
| 1216 | — | 1-CF₃, 3-F | F | F |
| 1217 | — | 1-F, 3-OCH₃ | F | F |
| 1218 | — | 1-OCH₃, 3-F | F | F |
| 1219 | — | 1-Br, 3-CF₃ | F | F |
| 1220 | — | 1-CF₃, 3-Br | F | F |
| 1221 | — | 1-Br, 3-OCH₃ | F | F |
| 1222 | — | 1-OCH₃, 3-Br | F | F |
| 1223 | — | 1-CF₃, 3-OCH₃ | F | F |
| 1224 | — | 1-OCH₃, 3-CF₃ | F | F |
| 1225 | CH₂ | 1-CH₃ | F | F |
| 1226 | CH₂ | 2-CH₃ | F | F |
| 1227 | CH₂ | 3-CH₃ | F | F |
| 1228 | CH₂ | 4-CH₃ | F | F |
| 1229 | CH₂ | 1-CH₃, 3-CH₃ | F | F |
| 1230 | CH₂ | 1-CH₃, 3-CH₃, 4-CH₃ | F | F |
| 1231 | CH₂ | 3-CH₃, 4-CH₃ | F | F |
| 1232 | CH₂ | 1-Cl | F | F |
| 1233 | CH₂ | 2-Cl | F | F |
| 1234 | CH₂ | 3-Cl | F | F |
| 1235 | CH₂ | 4-Cl | F | F |
| 1236 | CH₂ | 1-Cl, 3-Cl | F | F |
| 1237 | CH₂ | 1-Cl, 3-Cl, 4-Cl | F | F |
| 1238 | CH₂ | 3-Cl, 4-Cl | F | F |
| 1239 | CH₂ | 1-F | F | F |
| 1240 | CH₂ | 2-F | F | F |
| 1241 | CH₂ | 3-F | F | F |
| 1242 | CH₂ | 4-F | F | F |
| 1243 | CH₂ | 1-F, 3-F | F | F |
| 1244 | CH₂ | 1-F, 3-F, 4-F | F | F |
| 1245 | CH₂ | 3-F, 4-F | F | F |
| 1246 | CH₂ | 1-Br | F | F |
| 1247 | CH₂ | 2-Br | F | F |
| 1248 | CH₂ | 3-Br | F | F |
| 1249 | CH₂ | 4-Br | F | F |
| 1250 | CH₂ | 1-Br, 3-Br | F | F |
| 1251 | CH₂ | 1-Br, 3-Br, 4-Br | F | F |
| 1252 | CH₂ | 3-Br, 4-Br | F | F |
| 1253 | CH₂ | 1-CF₃ | F | F |
| 1254 | CH₂ | 2-CF₃ | F | F |
| 1255 | CH₂ | 3-CF₃ | F | F |
| 1256 | CH₂ | 4-CF₃ | F | F |
| 1257 | CH₂ | 1CF₃, 3-CF₃ | F | F |
| 1258 | CH₂ | 1-CF₃, 3-CF₃, 4-CF₃ | F | F |
| 1259 | CH₂ | 3-CF₃, 4-CF3 | F | F |
| 1260 | CH₂ | 1-OCH₃ | F | F |
| 1261 | CH₂ | 2-OCH₃ | F | F |
| 1262 | CH₂ | 3-OCH₃ | F | F |
| 1263 | CH₂ | 4-OCH₃ | F | F |
| 1264 | CH₂ | 1-OCH₃, 3-OCH₃ | F | F |
| 1265 | CH₂ | 1-OCH₃, 3-OCH₃, 4-OCH₃ | F | F |
| 1266 | CH₂ | 3,4-OCH₃, OCH₃ | F | F |
| 1267 | CH₂ | 1-CH₃, 3-Cl | F | F |
| 1268 | CH₂ | 1-Cl, 3-CH₃ | F | F |
| 1269 | CH₂ | 1-CH₃, 3-F | F | F |
| 1270 | CH₂ | 1-F, 3-CH₃ | F | F |
| 1271 | CH₂ | 1-CH₃, 3-Br | F | F |
| 1272 | CH₂ | 1-Br, 3-CH₃ | F | F |
| 1273 | CH₂ | 1-CH₃, 3-CF₃ | F | F |
| 1274 | CH₂ | 1-CF₃, 3-CH₃ | F | F |
| 1275 | CH₂ | 1-CH₃, 3OCH₃ | F | F |
| 1276 | CH₂ | 1-OCH₃, 3-CH₃ | F | F |
| 1277 | CH₂ | 1-Cl, 3-F | F | F |
| 1278 | CH₂ | 1-F, 3-Cl | F | F |
| 1279 | CH₂ | 1-Cl, 3-Br | F | F |
| 1280 | CH₂ | 1-Br, 3-Cl | F | F |
| 1281 | CH₂ | 1-Cl, 3-CF₃ | F | F |
| 1282 | CH₂ | 11-CF₃, 3-Cl | F | F |
| 1283 | CH₂ | 1-Cl, 3-OCH₃ | F | F |
| 1284 | CH₂ | 1-OCH₃, 3-Cl | F | F |
| 1285 | CH₂ | 1-F, 3-Br | F | F |
| 1286 | CH₂ | 1-Br, 3-F | F | F |
| 1287 | CH₂ | 1-F, 3-CF₃ | F | F |
| 1288 | CH₂ | 1-CF₃, 3-F | F | F |
| 1289 | CH₂ | 1-F, 3-OCH₃ | F | F |
| 1290 | CH₂ | 1-OCH₃, 3-F | F | F |
| 1291 | CH₂ | 1-Br, 3-CF₃ | F | F |
| 1292 | CH₂ | 1-CF₃, 3-Br | F | F |
| 1293 | CH₂ | 1-Br, 3-OCH₃ | F | F |
| 1294 | CH₂ | 1-OCH₃, 3-Br | F | F |
| 1295 | CH₂ | 1-CF₃, 3-OCH₃ | F | F |
| 1296 | CH₂ | 1-OCH₃, 3-CF₃ | F | F |

in table A the sign “—” has the meaning of B being a single bond;
the numbers in the definition of (R^{z1})ₙ indicates the position the radicals are attached to the aromatic ring.

Amongst compounds of the formulae (I.a) or (I.b), preference is also given to the compounds defined in the following tables 2 to 128:

Table 2: Compounds of the formulae (I.a) or (I.b) and their mixtures, wherein X is O, A is CH₂, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 3: Compounds of the formulae (I.a) or (I.b) and their mixtures, wherein X is NH, A is CH₂, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 4: Compounds of the formulae (I.a) or (I.b) and their mixtures, wherein X is NCH₃, A is CH₂, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 5: Compounds of the formulae (I.a) or (I.b) and their mixtures, wherein X is S, A is CH(C₆H₅), R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 6: Compounds of the formulae (I.a) or (I.b) and their mixtures, wherein X is O, A is CH(C₆H₅), R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 7: Compounds of the formulae (I.a) or (I.b) and their mixtures, wherein X is NH, A is CH(C₆H₅), R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 8: Compounds of the formulae (I.a) or (I.b) and their mixtures, wherein X is NCH₃, A is CH(C₆H₅), R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 9: Compounds of the formulae (I.a) or (I.b) and their mixtures, wherein X is S, A is O, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 10: Compounds of the formulae (I.a) or (I.b) and their mixtures, wherein X is O, A is O, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 11: Compounds of the formulae (I.a) or (I.b) and their mixtures, wherein X is NH, A is O, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 12: Compounds of the formulae (I.a) or (I.b) and their mixtures, wherein X is NCH₃, A is O, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 13: Compounds of the formulae (I.a) or (I.b) and their mixtures, wherein X is S, A is NH, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 14: Compounds of the formulae (I.a) or (I.b) and their mixtures, wherein X is O, A is NH, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 15: Compounds of the formulae (I.a) or (I.b) and their mixtures, wherein X is NH, A is NH, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 16: Compounds of the formulae (I.a) or (I.b) and their mixtures, wherein X is NCH_{, 3}A is NH, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 17: Compounds of the formulae (I.a) or (I.b) and their mixtures, wherein X is S, A is NCH₃, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 18: Compounds of the formulae (I.a) or (I.b) and their mixtures, wherein X is O, A is NCH₃, R¹ is hydrogen, R^{2a} or R^{2b} is hydrogen, R^{3a}, R^{3b}, R^{3c} and R^{3d} are hydrogen and

Table 19: Compounds of the formulae (I.a) or (I.b) and their mixtures, wherein X is NH, A is NCH₃, R¹ is hydrogen, R^{2a} or R^{2b} is hydrogen, R^{3a}, R^{3b}, R^{3c} and R^{3d} are hydrogen and wherein B, (R^{z1})R^{4a} and R^{4b} have the meanings given in each line of table A.

Table 20: Compounds of the formulae (I.a) or (I.b) and their mixtures, wherein X is NCH₃, A is NCH₃, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 21: Compounds of the formulae (I.a) or (I.b) and their mixtures, wherein X is S, A is S, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 22: Compounds of the formulae (I.a) or (I.b) and their mixtures, wherein X is O, A is S, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 23: Compounds of the formulae (I.a) or (I.b) and their mixtures, wherein X is NH, A is S, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 24: Compounds of the formulae (I.a) or (I.b) and their mixtures, wherein X is NCH₃, A is S, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 25: Compounds of the formulae (I.a) or (I.b) and their mixtures, wherein X is S, A is S(O), R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 26: Compounds of the formulae (I.a) or (I.b) and their mixtures, wherein X is O, A is S(O), R¹ is hydrogen, R^{2a} or R^{2b} is hydrogen, R^{3a}, R^{3b}, R^{3c} and R^{3d} are hydrogen and wherein B, (R^{z1})ₙ^{,} R^{4a} and R^{4b} have the meanings given in each line of table A. wherein B, (R^{z1})ₙ, R^{4a} and R^{4b} have the meanings given in each line of table A.

Table 27: Compounds of the formulae (I.a) or (I.b) and their mixtures, wherein X is NH, A is S(O), R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 28: Compounds of the formulae (I.a) or (I.b) and their mixtures, wherein X is NCH₃, A is S(O), R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 29: Compounds of the formulae (I.a) or (I.b) and their mixtures, wherein X is S, A is S(O)₂, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 30: Compounds of the formulae (I.a) or (I.b) and their mixtures, wherein X is O, A is S(O)₂, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 31: Compounds of the formulae (I.a) or (I.b) and their mixtures, wherein X is NH, A is S(O)₂, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 32: Compounds of the formulae (I.a) or (I.b) and their mixtures, wherein X is NCH₃, A is S(O)₂, R¹ is hydrogen, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 33: Compounds of the formulae (I.a) or (I.b) and their mixtures, wherein X is S, A is CH₂, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 34: Compounds of the formulae (I.a) or (I.b) and their mixtures, wherein X is O, A is CH₂, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given given in any of lines 145 to 1296 of table A.

Table 35: Compounds of the formulae (I.a) or (I.b) and their mixtures, wherein X is NH, A is CH₂, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 36: Compounds of the formulae (I.a) or (I.b) and their mixtures, wherein X is NCH₃, A is CH₂, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 37: Compounds of the formulae (I.a) or (I.b) and their mixtures, wherein X is S, A is CH(C₆H₅), R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 38: Compounds of the formulae (I.a) or (I.b) and their mixtures, wherein X is O, A is CH(C₆H₅), R¹ is hydrogen, R^{2a} or R^{2b} is CH₃, R^{3a}, R^{3b}, R^{3c} and R^{3d} are hydrogen wherein B, (R^{z1})ₙ, R^{4a} and R^{4b} have the meanings given in each line of table A.

Table 39: Compounds of the formulae (I.a) or (I.b) and their mixtures, wherein X is NH, A is CH(C₆H₅), R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 40: Compounds of the formulae (I.a) or (I.b) and their mixtures, wherein X is NCH₃, A is CH(C₆H₅), R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings giving in each line of table A.

Table 41: Compounds of the formulae (I.a) or (I.b) and their mixtures, wherein X is S, A is O, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 42: Compounds of the formulae (I.a) or (I.b) and their mixtures, wherein X is O, A is O, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 43: Compounds of the formulae (I.a) or (I.b) and their mixtures, wherein X is NH, A is O, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 44: Compounds of the formulae (I.a) or (I.b) and their mixtures, wherein X is NCH₃, A is O, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 45: Compounds of the formulae (I.a) or (I.b) and their mixtures, wherein X is S, A is NH, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 46: Compounds of the formulae (I.a) or (I.b) and their mixtures, wherein X is O, A is NH, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 47: Compounds of the formulae (I.a) or (I.b) and their mixtures, wherein X is NH, A is NH, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 48: Compounds of the formulae (I.a) or (I.b) and their mixtures, wherein X is NCH₃, A is NH, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 49: Compounds of the formulae (I.a) or (I.b) and their mixtures, wherein X is S, A is NCH₃, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 50: Compounds of the formulae (I.a) or (I.b) and their mixtures, wherein X is O, A is NCH₃, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 51: Compounds of the formulae (I.a) or (I.b) and their mixtures, wherein X is NH, A is NCH₃, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 52: Compounds of the formulae (I.a) or (I.b) and their mixtures, wherein X is NCH₃, A is NCH₃, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 53: Compounds of the formulae (I.a) or (I.b) and their mixtures, wherein X is S, A is S, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 54: Compounds of the formulae (I.a) or (I.b) and their mixtures, wherein X is O, A is S, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 55: Compounds of the formulae (I.a) or (I.b) and their mixtures, wherein X is NH, A is S, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 56: Compounds of the formulae (I.a) or (I.b) and their mixtures, wherein X is NCH₃, A is S, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 57: Compounds of the formulae (I.a) or (I.b) and their mixtures, wherein X is S, A is S(O), R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 58: Compounds of the formulae (I.a) or (I.b) and their mixtures, wherein X is O, A is S(O), R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 59: Compounds of the Formulae (I.a) or (I.b) and their mixtures, wherein X is NH, A is S(O), R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 60: Compounds of the formulae (I.a) or (I.b) and their mixtures, wherein X is NCH₃, A is S(O), R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 61: Compounds of the formulae (I.a) or (I.b) and their mixtures, wherein X is S, A is S(O)₂, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 62: Compounds of the formulae (I.a) or (I.b) and their mixtures, wherein X is O, A is S(O)₂, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 63: Compounds of the formulae (I.a) or (I.b) and their mixtures, wherein X is NH, A is S(O)₂, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 64: Compounds of the formulae (I.a) or (I.b) and their mixtures, wherein X is NCH₃, A is S(O)₂, R¹ is hydrogen, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 65: Compounds of the formulae (I.a) or (I.b) and their mixtures, wherein X is S, A is CH₂, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 66: Compounds of the formulae (I.a) or (I.b) and their mixtures, wherein X is O, A is CH₂, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 67: Compounds of the formulae (I.a) or (I.b) and their mixtures, wherein X is NH, A is CH₂, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 68: Compounds of the formulae (I.a) or (I.b) and their mixtures, wherein X is NCH₃, A is CH₂, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 69: Compounds of the formulae (I.a) or (I.b) and their mixtures, wherein X is S, A is CH(C₆H₅), R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 70: Compounds of the formulae (I.a) or (I.b) and their mixtures, wherein X is O, A is CH(C₆H₅), R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 71: Compounds of the formulae (I.a) or (I.b) and their mixtures, wherein X is NH, A is CH(C₆H₅), R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 72: Compounds of the formulae (I.a) or (I.b) and their mixtures, wherein X is NCH₃, A is CH(C₆H₅), R¹ is hydrogen, R^{2a} or R^{2b} is C(O)CH₃, R^{3a}, R^{3b}, R^{3c} and R^{3c} are hydrogen and wherein B, (R^{z1})ₙ, R^{4a} and R^{4b} have the meanings given in each line of table A.

Table 73: Compounds of the formulae (I.a) or (I.b) and their mixtures, wherein X is S, A is O, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 74: Compounds of the formulae (I.a) or (I.b) and their mixtures, wherein X is O, A is O, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 75: Compounds of the formulae (I.a) or (I.b) and their mixtures, wherein X is NH, A is O, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 76: Compounds of the formulae (I.a) or (I.b) and their mixtures, wherein X is NCH₃, A is O, R¹ is hydrogen, R^{2a} or R^{2b} is C(O)CH₃, R^{3a}, R^{3b}, R^{3c} and R^{3d} are hydrogen R^{4a} and R^{4b} have the meanings given in each line of table A.

Table 77: Compounds of the formulae (I.a) or (I.b) and their mixtures, wherein X is S, A is NH, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 78: Compounds of the formulae (I.a) or (I.b) and their mixtures, wherein X is O, A is NH, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 79: Compounds of the formulae (I.a) or (I.b) and their mixtures, wherein X is NH, A is NH, R¹ is hydrogen, R^{2a} or R^{2b} is C(O)CH₃, R^{3a}, R^{3b}, R^{3c} and R^{3d} are hydrogen an wherein B, (R^{z1})ₙ, R^{4a} and R^{4b} have the meanings given in each line of table A.

Table 80: Compounds of the formulae (I.a) or (I.b) and their mixtures, wherein X is NCH₃, A is NH, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 81: Compounds of the formulae (I.a) or (I.b) and their mixtures, wherein X is S, A is NCH₃, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 82: Compounds of the formulae (I.a) or (I.b) and their mixtures, wherein X is O, A is NCH₃, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 83: Compounds of the formulae (I.a) or (I.b) and their mixtures, wherein X is NH, A is NCH₃, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 84: Compounds of the formulae (I.a) or (I.b) and their mixtures, wherein X is NCH₃, A is NCH₃, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 85: Compounds of the formulae (I.a) or (I.b) and their mixtures, wherein X is S, A is S, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 86: Compounds of the formulae (I.a) or (I.b) and their mixtures, wherein X is O, A is S, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 87: Compounds of the formulae (I.a) or (I.b) and their mixtures, wherein X is NH, A is S, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 88: Compounds of the formulae (I.a) or (I.b) and their mixtures, wherein X is NCH₃, A is S, R¹ is hydrogen, R^{2a} or R^{2b} is C(O)CH₃, R^{3a}, R^{3b}, R^{3c} and R^{3d} are hydrogent and wherein B, (R^{z1})ₙ, R^{4a} and R^{4b} have the meanings given in each line of table A.

Table 89: Compounds of the formulae (I.a) or (I.b) and their mixtures, wherein X is S, A is S(O), R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings in each line of table A.

Table 90: Compounds of the formulae (I.a) or (I.b) and their mixtures, wherein X is O, A is S(O), R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 91: Compounds of the formulae (I.a) or (I.b) and their mixtures, wherein X is NH, A is S(O), R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 92: Compounds of the formulae (I.a) or (I.b) and their mixtures, wherein X is NCH₃, A is S(O), R¹ is hydrogen, R^{2a} or R^{2b} is C(O)CH₃, R^{3a}, R^{3b}, R^{3c} and R^{3d} are hydrogen drogen and wherein B, (R^{z1})ₙ, R^{4a} and R^{4b} have the meanings given in each line of table A.

Table 93: Compounds of the formulae (I.a) or (I.b) and their mixtures, wherein X is S, A is S(O)₂, R¹ is hydrogen, R^{2a} or R^{2b} is C(O)CH₃, R^{3a}, R^{3b}, R^{3c} and R^{3d} are hydrogent and wherein B, (R^{z1})ₙ, R^{4a} and R^{4b} have the meanings given in each line of table A.

Table 94: Compounds of the formulae (I.a) or (I.b) and their mixtures, wherein X is O, A is S(O)₂, R¹ is hydrogen, R^{2a} or R^{2b} is C(O)CH₃, R^{3a}, R^{3b}, R^{3c} and R^{3d} are hydrogent and wherein B, (R^{z1})ₙ, R^{4a} and R^{4b} have the meanings given in each line of table A.

Table 95: Compounds of the formulae (I.a) or (I.b) and their mixtures, wherein X is NH, A is S(O)₂, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 96: Compounds of the formulae (I.a) or (I.b) and their mixtures, wherein X is NCH₃, A is S(O)₂, R¹ is hydrogen, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 97: Compounds of the formulae (I.a) or (I.b) and their mixtures, wherein X is S, A is CH₂, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 98: Compounds of the formulae (I.a) or (I.b) and their mixtures, wherein X is O, A is CH₂, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 99: Compounds of the formulae (I.a) or (I.b) and their mixtures, wherein X is NH, A is CH₂, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 100: Compounds of the formulae (I.a) or (I.b) and their mixtures, wherein X is NCH₃, A is CH₂, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in any of lines 145 to 1296 of table A.

Table 101: Compounds of the formulae (I.a) or (I.b) and their mixtures, wherein X is S, A is CH(C₆H₅), R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 102: Compounds of the formulae (I.a) or (I.b) and their mixtures, wherein X is O, A is CH(C₆H₅), R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 103: Compounds of the formulae (I.a) or (I.b) and their mixtures, wherein X is NH, A is CH(C₆H₅), R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 104: Compounds of the formulae (I.a) or (I.b) and their mixtures, wherein X is NCH₃, A is CH(C₆H₅), R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 105: Compounds of the formulae (I.a) or (I.b) and their mixtures, wherein X is S, A is O, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 106: Compounds of the formulae (I.a) or (I.b) and their mixtures, wherein X is O, A is O, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 107: Compounds of the formulae (I.a) or (I.b) and their mixtures, wherein X is NH, A is O, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 108: Compounds of the formulae (I.a) or (I.b) and their mixtures, wherein X is NCH₃, A is O, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 109: Compounds of the formulae (I.a) or (I.b) and their mixtures, wherein X is S, A is NH, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 110: Compounds of the formulae (I.a) or (I.b) and their mixtures, wherein X is O, A is NH, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 111: Compounds of the formulae (I.a) or (I.b) and their mixtures, wherein X is NH, A is NH, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 112: Compounds of the formulae (I.a) or (I.b) and their mixtures, wherein X is NCH₃, A is NH, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 113: Compounds of the formulae (I.a) or (I.b) and their mixtures, wherein X is S, A is NCH₃, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 114: Compounds of the formulae (I.a) or (I.b) and their mixtures, wherein X is O, A is NCH₃, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 115: Compounds of the formulae (I.a) or (I.b) and their mixtures, wherein X is NH, A is NCH₃, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 116: Compounds of the formulae (I.a) or (I.b) and their mixtures, wherein X is NCH₃, A is NCH₃, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 117: Compounds of the formulae (I.a) or (I.b) and their mixtures, wherein X is S, A is S, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 118: Compounds of the formulae (I.a) or (I.b) and their mixtures, wherein X is O, A is S, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 119: Compounds of the formulae (I.a) or (I.b) and their mixtures, wherein X is NH, A is S, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 120: Compounds of the formulae (I.a) or (I.b) and their mixtures, wherein X is NCH₃, A is S, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 121: Compounds of the formulae (I.a) or (I.b) and their mixtures, wherein X is S, A is S(O), R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 122: Compounds of the formulae (I.a) or (I.b) and their mixtures, wherein X is O, A is S(O), R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 123: Compounds of the formulae (I.a) or (I.b) and their mixtures, wherein X is NH, A is S(O), R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 124: Compounds of the formulae (I.a) or (I.b) and their mixtures, wherein X is NCH₃, A is S(O), R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 125: Compounds of the formulae (I.a) or (I.b) and their mixtures, wherein X is S, A is S(O)₂, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 126: Compounds of the formulae (I.a) or (I.b) and their mixtures, wherein X is O, A is S(O)₂, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 127: Compounds of the formulae (I.a) or (I.b) and their mixtures, wherein X is NH, A is S(O)₂, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 128: Compounds of the formulae (I.a) or (I.b) and their mixtures, wherein X is NCH₃, A is S(O)₂, R¹ is hydrogen, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 129: Compounds of the formulae (I.a) or (I.b) and their mixtures, wherein X is S, A is CH₂, R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 130: Compounds of the formulae (I.a) or (I.b) and their mixtures, wherein X is O, A is CH₂, R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 131: Compounds of the formulae (I.a) or (I.b) and their mixtures, wherein X is NH, A is CH₂, R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in any each line of table A.

Table 132: Compounds of the formulae (I.a) or (I.b) and their mixtures, wherein X is NCH₃, A is CH₂, R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 133: Compounds of the formulae (I.a) or (I.b) and their mixtures, wherein X is S, A is CH(C₆H₅), R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 134: Compounds of the formulae (I.a) or (I.b) and their mixtures, wherein X is O, A is CH(C₆H₅), R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 135: Compounds of the formulae (I.a) or (I.b) and their mixtures, wherein X is NH, A is CH(C₆H₅), R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 136: Compounds of the formulae (I.a) or (I.b) and their mixtures, wherein X is NCH₃, A is CH(C₆H₅), R¹ is CH₃, R^{2a} or R^{2b} is hydrogen, R^{3a}, R^{3b}, R^{3c} , and R^{3d} are hydrogent and wherein B, (R^{z1})ₙ, R^{4a} and R^{4b} have the meanings given in each line of table A.

Table 137: Compounds of the formulae (I.a) or (I.b) and their mixtures, wherein X is S, A is O, R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 138: Compounds of the formulae (I.a) or (I.b) and their mixtures, wherein X is O, A is O, R¹ is CH₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 139: Compounds of the formulae (I.a) or (I.b) and their mixtures, wherein X is NH, A is O, R¹ is CH³, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 140: Compounds of the formulae (I.a) or (I.b) and their mixtures, wherein X is NCH₃, A is O, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 141: Compounds of the formulae (I.a) or (I.b) and their mixtures, wherein X is S, A is NH, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 142: Compounds of the formulae (I.a) or (I.b) and their mixtures, wherein X is O, A is NH, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 143: Compounds of the formulae (I.a) or (I.b) and their mixtures, wherein X is NH, A is NH, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 144: Compounds of the formulae (I.a) or (I.b) and their mixtures, wherein X is NCH₃, A is NH, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 145: Compounds of the formulae (I.a) or (I.b) and their mixtures, wherein X is S, A is NCH₃, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 146: Compounds of the formulae (I.a) or (I.b) and their mixtures, wherein X is O, A is NCH₃, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 147: Compounds of the formulae (I.a) or (I.b) and their mixtures, wherein X is NH, A is NCH₃, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 148: Compounds of the formulae (I.a) or (I.b) and their mixtures, wherein X is NCH₃, A is NCH₃, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 149: Compounds of the formulae (I.a) or (I.b) and their mixtures, wherein X is S, A is S, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 150: Compounds of the formulae (I.a) or (I.b) and their mixtures, wherein X is O, A is S, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 151: Compounds of the formulae (I.a) or (I.b) and their mixtures, wherein X is NH, A is S, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 152: Compounds of the formulae (I.a) or (I.b) and their mixtures, wherein X is NCH₃, A is S, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 153: Compounds of the formulae (I.a) or (I.b) and their mixtures, wherein X is S, A is S(O), R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 154: Compounds of the formulae (I.a) or (I.b) and their mixtures, wherein X is O, A is S(O), R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 155: Compounds of the formulae (I.a) or (I.b) and their mixtures, wherein X is NH, A is S(O), R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 156: Compounds of the formulae (I.a) or (I.b) and their mixtures, wherein X is NCH₃, A is S(O), R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 157: Compounds of the formulae (I.a) or (I.b) and their mixtures, wherein X is S, A is S(O)₂, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 158: Compounds of the formulae (I.a) or (I.b) and their mixtures, wherein X is O, A is S(O)₂, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 159: Compounds of the formulae (I.a) or (I.b) and their mixtures, wherein X is NH, A is S(O)₂, R¹ is C₃, R^{2a} or R^{2b} is hydrogen, R^{3a}, R^{3b}, R^{3c} and R^{3d} are hydrogen and wherein B, (R^{z1})ₙ, R^{4a} and R^{4b} have the meanings given in each line of table A.

Table 160: Compounds of the formulae (I.a) or (I.b) and their mixtures, wherein X is NCH₃, A is S(O)₂, R¹ is C₃, R^{2a} or R^{2b} is hydrogen, R^{3a}, R^{3b}, R^{3c} and R^{3d} are hydrogent and wherein B, (R^{z1})ₙ, R^{4a} and R^{4b} have the meanings given in each line of table A.

Table 161: Compounds of the formulae (I.a) or (I.b) and their mixtures, wherein X is S, A is CH₂, R¹ is C₃, R^{2a} or R^{2b} is CH₃, R^{3a}, R^{3b}, R^{3c} and R^{3d} are hydrogen and B, (R^{z1})ₙ, R^{4a} and R^{4b} have the meanings given in each line of table A.

Table 162: Compounds of the formulae (I.a) or (I.b) and their mixtures, wherein X is O, A is CH₂, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 163: Compounds of the formulae (I.a) or (I.b) and their mixtures, wherein X is NH, A is CH₂, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 164: Compounds of the formulae (I.a) or (I.b) and their mixtures, wherein X is NCH₃, A is CH₂, R¹ is C₃, R^{2a} or R^{2b} is CH₃, R^{3a}, R^{3b}, R^{3c} and R^{3d} are hydrogent and wherein B, (R^{z1})ₙ, R^{4a} and R^{4b} have the meanings given in each line of table A.

Table 165: Compounds of the formulae (I.a) or (I.b) and their mixtures, wherein X is S, A is CH(C₆H₅), R¹ is C₃, R^{2a} or R^{2b} is CH₃, R^{3a}, R^{3b}, R^{3c} and R^{3d} are hydrogent and wherein B, (R^{z1})ₙ, R^{4a} and R^{4b} have the meanings given in each line of table A.

Table 166: Compounds of the formulae (I.a) or (I.b) and their mixtures, wherein X is O, A is CH(C₆H₅), R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 167: Compounds of the formulae (I.a) or (I.b) and their mixtures, wherein X is NH, A is CH(C₆H₅), R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 168: Compounds of the formulae (I.a) or (I.b) and their mixtures, wherein X is NCH₃, A is CH(C₆H₅), R¹ is C₃, R^{2a} or R^{2b} is CH₃, R^{3a}, R^{3b}, R^{3c} and R^{3d} are hydrogent and wherein B, (R^{z1})ₙ, R^{4a} and R^{4b} have the meanings given in each line of table A.

Table 169: Compounds of the formulae (I.a) or (I.b) and their mixtures, wherein X is S, A is O, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 170: Compounds of the formulae (I.a) or (I.b) and their mixtures, wherein X is O, A is O, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 171: Compounds of the formulae (I.a) or (I.b) and their mixtures, wherein X is NH, A is O, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 172: Compounds of the formulae (I.a) or (I.b) and their mixtures, wherein X is NCH₃, A is O, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 173: Compounds of the formulae (I.a) or (I.b) and their mixtures, wherein X is S, A is NH, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 174: Compounds of the formulae (I.a) or (I.b) and their mixtures, wherein X is O, A is NH, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 175: Compounds of the formulae (I.a) or (I.b) and their mixtures, wherein X is NH, A is NH, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 176: Compounds of the formulae (I.a) or (I.b) and their mixtures, wherein X is NCH₃, A is NH, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 177: Compounds of the formulae (I.a) or (I.b) and their mixtures, wherein X is S, A is NCH₃, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 178: Compounds of the formulae (I.a) or (I.b) and their mixtures, wherein X is O, A is NCH₃, R¹ is C₃, R^{2a} or R^{2b} is CH₃, R^{3a}, R^{3b}, R^{3c} and R^{3d} are hydrogen and B, (R^{z1})ₙ, R^{4a} and R^{4b} have the meanings given in each line of table A.

Table 179: Compounds of the formulae (I.a) or (I.b) and their mixtures, wherein X is NH, A is NCH₃, R¹ is CH³, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 180: Compounds of the formulae (I.a) or (I.b) and their mixtures, wherein X is NCH₃, A is NCH₃, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 181: Compounds of the formulae (I.a) or (I.b) and their mixtures, wherein X is S, A is S, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 182: Compounds of the formulae (I.a) or (I.b) and their mixtures, wherein X is O, A is S, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 183: Compounds of the formulae (I.a) or (I.b) and their mixtures, wherein X is NH, A is S, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 184: Compounds of the formulae (I.a) or (I.b) and their mixtures, wherein X is NCH₃, A is S, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 185: Compounds of the formulae (I.a) or (I.b) and their mixtures, wherein X is S, A is S(O), R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 186: Compounds of the formulae (I.a) or (I.b) and their mixtures, wherein X is O, A is S(O), R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 187: Compounds of the formulae (I.a) or (I.b) and their mixtures, wherein X is NH, A is S(O), R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 188: Compounds of the formulae (I.a) or (I.b) and their mixtures, wherein X is NCH₃, A is S(O), R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 189: Compounds of the formulae (I.a) or (I.b) and their mixtures, wherein X is S, A is S(O)₂, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 190: Compounds of the formulae (I.a) or (I.b) and their mixtures, wherein X is O, A is S(O)₂R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 191: Compounds of the formulae (I.a) or (I.b) and their mixtures, wherein X is NH, A is S(O)₂, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 192: Compounds of the formulae (I.a) or (I.b) and their mixtures, wherein X is NCH₃, A is S(O)₂, R¹ is C₃, R^{2a} or R^{2b} is CH₃, R^{3a}, R^{3b}, R^{3c} and R^{3d} are hydrogen and wherein B, (R^{z1})ₙ, R^{4a} and R^{4b} have the meanings given in each line of table A.

Table 193: Compounds of the formulae (I.a) or (I.b) and their mixtures, wherein X is S, A is CH₂, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 194: Compounds of the formulae (I.a) or (I.b) and their mixtures, wherein X is O, A is CH₂, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 195: Compounds of the formulae (I.a) or (I.b) and their mixtures, wherein X is NH, A is CH₂, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 196: Compounds of the formulae (I.a) or (I.b) and their mixtures, wherein X is NCH₃, A is CH₂, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 197: Compounds of the formulae (I.a) or (I.b) and their mixtures, wherein X is S, A is CH(C₆H₅), R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 198: Compounds of the formulae (I.a) or (I.b) and their mixtures, wherein X is O, A is CH(C₆H₅), R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 199: Compounds of the formulae (I.a) or (I.b) and their mixtures, wherein X is NH, A is CH(C₆H₅), R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 200: Compounds of the formulae (I.a) or (I.b) and their mixtures, wherein X is NCH₃, A is CH(C₆H₅), R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 201: Compounds of the formulae (I.a) or (I.b) and their mixtures, wherein X is S, A is O, R¹ is CH₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 202: Compounds of the formulae (I.a) or (I.b) and their mixtures, wherein X is O, A is O, R¹ is C₃, R^{2a} or R^{2b} is C(O)CH₃, R^{3a}, R^{3b}, R^{3c} and R^{3d} are hydrogen wherein B, (R^{z1})ₙ, R^{4a} and R^{4b} have the meanings given in each line of table A.

Table 203: Compounds of the formulae (I.a) or (I.b) and their mixtures, wherein X is NH, A is O, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 204: Compounds of the formulae (I.a) or (I.b) and their mixtures, wherein X is NCH₃, A is O, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 205: Compounds of the formulae (I.a) or (I.b) and their mixtures, wherein X is S, A is NH, R¹ is CH₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 206: Compounds of the formulae (I.a) or (I.b) and their mixtures, wherein X is O, A is NH, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 207: Compounds of the formulae (I.a) or (I.b) and their mixtures, wherein X is NH, A is NH, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 208: Compounds of the formulae (I.a) or (I.b) and their mixtures, wherein X is NCH₃, A is NH, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 209: Compounds of the formulae (I.a) or (I.b) and their mixtures, wherein X is S, A is NCH₃, R¹ is CH₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 210: Compounds of the formulae (I.a) or (I.b) and their mixtures, wherein X is O, A is NCH₃, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 211: Compounds of the formulae (I.a) or (I.b) and their mixtures, wherein X is NH, A is NCH₃, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 212: Compounds of the formulae (I.a) or (I.b) and their mixtures, wherein X is NCH₃, A is NCH₃, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 213: Compounds of the formulae (I.a) or (I.b) and their mixtures, wherein X is S, A is S, R¹ is CH₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 214: Compounds of the formulae (I.a) or (I.b) and their mixtures, wherein X is O, A is S, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 215: Compounds of the formulae (I.a) or (I.b) and their mixtures, wherein X is NH, A is S, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 216: Compounds of the formulae (I.a) or (I.b) and their mixtures, wherein X is NCH₃, A is S, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 217: Compounds of the formulae (I.a) or (I.b) and their mixtures, wherein X is S, A is S(O), R¹ is CH₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 218: Compounds of the formulae (I.a) or (I.b) and their mixtures, wherein X is O, A is S(O), R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 219: Compounds of the formulae (I.a) or (I.b) and their mixtures, wherein X is NH, A is S(O), R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 220: Compounds of the formulae (I.a) or (I.b) and their mixtures, wherein X is NCH₃, A is S(O), R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 221: Compounds of the formulae (I.a) or (I.b) and their mixtures, wherein X is S, A is S(O)₂, R¹ is CH₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 222: Compounds of the formulae (I.a) or (I.b) and their mixtures, wherein X is O, A is S(O)₂, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 223: Compounds of the formulae (I.a) or (I.b) and their mixtures, wherein X is NH, A is S(O)₂, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 224: Compounds of the formulae (I.a) or (I.b) and their mixtures, wherein X is NCH₃, A is S(O)₂, R¹ is C₃, R^{2a} or R^{2b} is C(O)CH₃, R^{3a}, R^{3b}, R^{3c} and R^{3d} are hydrogen and wherein B, (R^{z1})ₙ, R^{4a} and R^{4b} have the meanings given in each line of table A.

Table 225: Compounds of the formulae (I.a) or (I.b) and their mixtures, wherein X is S, A is CH₂, R¹ is CH₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 226: Compounds of the formulae (I.a) or (I.b) and their mixtures, wherein X is O, A is CH₂, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 227: Compounds of the formulae (I.a) or (I.b) and their mixtures, wherein X is NH, A is CH₂, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 228: Compounds of the formulae (I.a) or (I.b) and their mixtures, wherein X is NCH₃, A is CH₂, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 229: Compounds of the formulae (I.a) or (I.b) and their mixtures, wherein X is S, A is CH(C₆H₅), R¹ is CH₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 230: Compounds of the formulae (I.a) or (I.b) and their mixtures, wherein X is O, A is CH(C₆H₅), R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 231: Compounds of the formulae (I.a) or (I.b) and their mixtures, wherein X is NH, A is CH(C₆H₅), R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 232: Compounds of the formulae (I.a) or (I.b) and their mixtures, wherein X is NCH₃, A is CH(C₆H₅), R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 233: Compounds of the formulae (I.a) or (I.b) and their mixtures, wherein X is S, A is O, R¹ is CH₃, R^{2a} or R^{2b} is CN, R^{3a}, R^{3b}, R^{ac} and R^{3d} are hydrogen and wherein B, (R^{z1})ₙ, R^{4a} and R^{4b} have the meanings given in each line of table A.

Table 234: Compounds of the formulae (I.a) or (I.b) and their mixtures, wherein X is O, A is O, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 235: Compounds of the formulae (I.a) or (I.b) and their mixtures, wherein X is NH, A is O, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 236: Compounds of the formulae (I.a) or (I.b) and their mixtures, wherein X is NCH₃, A is O, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 237: Compounds of the formulae (I.a) or (I.b) and their mixtures, wherein X is S, A is NH, R¹ is CH₃, R^{2a} or R^{2b} is CN, R^{3a}, R^{3b}, R^{ac} and R^{3d} are hydrogen and wherein B,

Table 238: Compounds of the formulae (I.a) or (I.b) and their mixtures, wherein X is O, A is NH, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 239: Compounds of the formulae (I.a) or (I.b) and their mixtures, wherein X is NH, A is NH, R¹ is C₃, R^{2a} or R^{2b} is CN, R^{3a}, R^{3b}, R^{ac} and R^{3d} are hydrogen and wherein B, (R^{z1})ₙ, R^{4a} and R^{4b} have the meanings given in each line of table A.

Table 240: Compounds of the formulae (I.a) or (I.b) and their mixtures, wherein X is NCH₃, A is NH, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 241: Compounds of the formulae (I.a) or (I.b) and their mixtures, wherein X is S, A is NCH₃, R¹ is CH₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 242: Compounds of the formulae (I.a) or (I.b) and their mixtures, wherein X is O, A is NCH₃, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 243: Compounds of the formulae (I.a) or (I.b) and their mixtures, wherein X is NH, A is NCH₃, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 244: Compounds of the formulae (I.a) or (I.b) and their mixtures, wherein X is NCH₃, A is NCH₃, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 245: Compounds of the formulae (I.a) or (I.b) and their mixtures, wherein X is S, A is S, R¹ is CH₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 246: Compounds of the formulae (I.a) or (I.b) and their mixtures, wherein X is O, A is S, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 247: Compounds of the formulae (I.a) or (I.b) and their mixtures, wherein X is NH, A is S, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 248: Compounds of the formulae (I.a) or (I.b) and their mixtures, wherein X is NCH₃, A is S, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 249: Compounds of the formulae (I.a) or (I.b) and their mixtures, wherein X is S, A is S(O), R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 250: Compounds of the formulae (I.a) or (I.b) and their mixtures, wherein X is O, A is S(O), R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 251: Compounds of the formulae (I.a) or (I.b) and their mixtures, wherein X is NH, A is S(O), R¹ is C₃, Rea or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 252: Compounds of the formulae (I.a) or (I.b) and their mixtures, wherein X is NCH₃, A is S(O), R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 253: Compounds of the formulae (I.a) or (I.b) and their mixtures, wherein X is S, A is S(O)₂, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 254: Compounds of the formulae (I.a) or (I.b) and their mixtures, wherein X is O, A is S(O)₂, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 255: Compounds of the formulae (I.a) or (I.b) and their mixtures, wherein X is NH, A is S(O)₂, R¹ is C₃, R^{2a} or R^{2b} is CN, R^{3a}, R^{3b}, R^{3c} and R^{3d} are hydrogen and wherein B, (R^{z1})ₙ, R^{4a} and R^{4b} have the meanings given in each line of table A.

Table 256: Compounds of the formulae (I.a) or (I.b) and their mixtures, wherein X is NCH₃, A is S(O)₂, R¹ is C₃, R^{2a} or R^{2b} is CN, R^{3a}, R^{3b}, R^{3c} and R^{3d} are wherein B, (R^{z1})ₙ, R^{4a} and R^{4b} have the meanings given in each line of table A.

Tables 257 to 384: compounds of formula (I.a) or (I.b) and their mixtures which correspond to the compounds of tables 129 to 256, except that R¹ is phenyl instead of CH₃.

Tables 385 to 512: compounds of formula (I.a) or (I.b) and their mixtures which correspond to the compounds of tables 129 to 256, except that R¹ is benzyl instead of CH₃.

Tables 513 to 640: compounds of formula (I.a) or (I.b) and their mixtures which correspond to the compounds of tables 129 to 256, except that R¹ is allyl instead of CH₃.

The compounds of formulae (I.a) and (I.b) can be obtained as outlined in schemes 1 to 4.

The compounds of the formula (I.a) according to the invention wherein X is oxygen or sulphur and R^{2a} is hydrogen (referred herein as compounds (I.A1)) can be prepared e.g. from the corresponding urea compounds (III) and thiourea compounds (II), respectively, as shown in scheme 1.

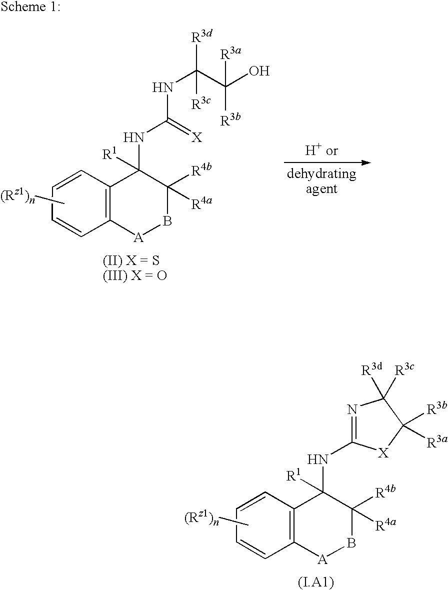

wherein A, B, (R^{z1})ₙ, R¹, R^{3a}, R^{3b}, R^{3c}, R^{3d}, R^{4a} and R^{4b} have the meaning given above.

The thiourea compound (II) and the urea compound (III), respectively, can be cyclized by conventional means thereby obtaining the azoline compound of the formula (I.A1). Cyclization of the compound (II) and (III), respectively, can be achieved e.g. under acid catalysis or under dehydrating conditions e.g. by Mitsunobu's reaction (see Tetrahedron Letters 1999, 40, 3125-3128).

Alternatively, the compounds of the formula (I.a) according to the invention wherein X is O or S, R^{2a}, R^{3a}, R^{3b}, R^{3c} and R^{3d} are hydrogen (referred herein as compounds (I.A2)) can be prepared by the method shown in scheme 2.

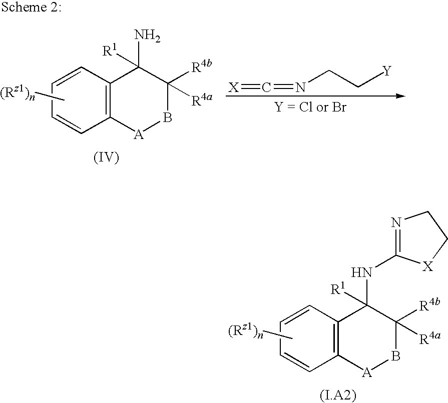

wherein A, B, (R^{z1})ₙ, R¹, R^{4a} and R^{4b} are as defined above.

An amine (IV) or a salt thereof can be converted to an azoline (I.A2) by reaction with 2-chloroethylisothiocyanate or 2-chloroethylisocyanate (e.g. as described in Bioorg. Med. Chem. Lett. 1994, 4, 2317-22) and subsequent cyclization in the presence or absence of a base.

1-Chloro-2-isothiocyanatoethane (CAS 6099-88-3), 2-chloroethylisocyanate (CAS 1943-83-5), 1-bromo-2-isothiocyanatoethane (CAS 1483-41-6) and 2-bromoethylisocyanate (CAS 42865-19-0) are commercially available.

Compounds of the formula (I.a) according to the invention wherein X is NR⁵ (referred to herein as compounds (I.A3)) may be prepared by the method outlined in scheme 3.

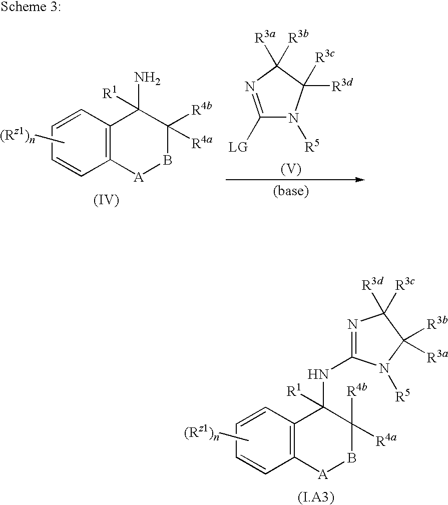

wherein (R^{z1})ₙ, A, B, R¹, R^{3a}, R^{3b}, R^{3c}, R^{3d}, R^{4a}, R^{4b} and R⁵ are as defined above and LG is a leaving group.

Compounds of the formula (I.A3) may be obtained by reacting an appropriate substituted amine (IV) or a salt thereof with a 2-substituted imidazoline (V) in an appropriate solvent. This reaction can be carried out, for example analogous to the methods described in U.S. Pat. No. 5,130,441 or EP 0389765.

Amines (IV) are known in the art or can be prepared by methods familiar to an organic chemist, for instance by application of general methods for the synthesis of amines. Suitable amine salts (IV) are e.g. the acid addition salts formed by treating an amine (IV) with an inorganic or organic acid. Anions of useful acids are e.g. sulfate, hydrogen sulfate, phosphate, dihydrogen phosphate, hydrogen phosphate, nitrate, bicarbonate, carbonate, chloride, bromide, p-toluene sulfonate, and the anions of C₁-C₄-alkanoic acids such as acetate, propionate, and the like.

For instance, amines (IV) can be prepared from suitable ketones (VI) by the methods depicted in schemes 4 and 5 below.

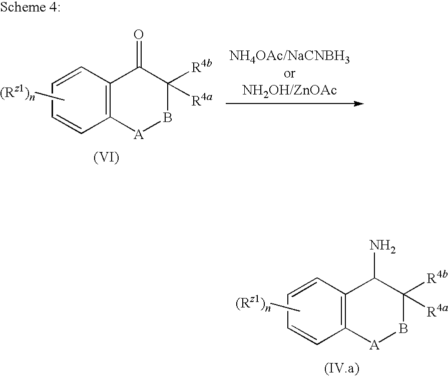

Amines (IV) wherein R¹ is hydrogen (referred to as compounds (IV.a)) can be obtained through reductive amination using e. g. NH₄OAc and NaCNBH₃ or NH₂OH/ZnOAc (see R. C. Larock, Comprehensive Organic Transformations 2^{nd} Ed., Wiley-VCH, 1999, p. 843-846). Alternatively compounds (IV.a) can be prepared by reduction of the ketone and subsequent amination of the resulting alcohol (see Mitsunobu, Hughes, Organic Reactions 1992, 42, 335-656). Compounds (IV.a) wherein A is O or S can be prepared in analogues manner to the method described in Turan-Zitouni et al. Turk. Farmaco, Edizione Scientifica (1988), 43, 643-55 or P. Sebok et al. Heterocyclic Communications, 1998, 4, 547-557.

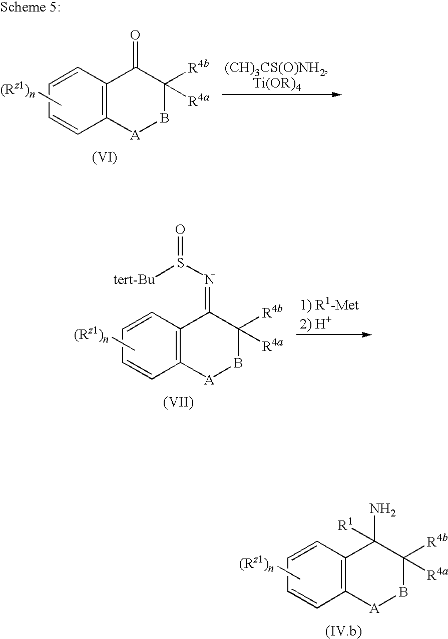

Amines (IV) wherein R¹ is different from hydrogen (referred to as compounds (IV.b)) can be obtained from the ketone (VI) via a two step synthesis. In a first step the ketone is reacted with a suitable sulfinamide such as (2-methyl-2-propane)sulfinamide in presence of a Lewis acid such as titanium tetraalkylate. The radical R¹ is subsequently introduced via a nucleophilic addition of some metallorganic compound followed by protonation and deprotection of the amino group.

Suitable ketones (VI) are known in the art or can be prepared by methods familiar to an organic chemist, for instance by application of general methods for the synthesis of ketones. Schemes 6 and 7 below illustrate some synthetic routes towards substituted ketones (VI).

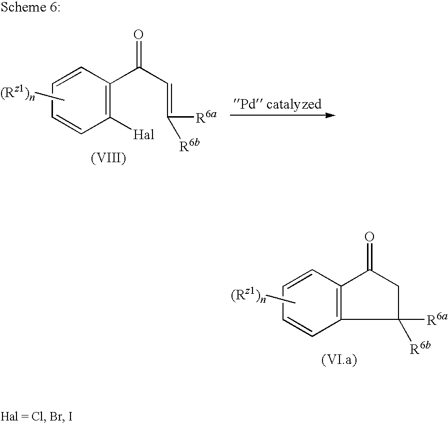

For instance ketones (VI) wherein A is C(R^{6a})(R^{6b}) and B is a chemical bond (referred to as compounds (VI.a)) can be obtained from a suitable halogenated phenyl (VIII) via a palladium-catalyzed Heck-type cyclization. The preparation of 3-substituted Indanones is for instance described in A. Pueschl, H. C. Rudbeck, A. Faldt, A. Confante, J. Kehler Synthesis 2005, 291-295.

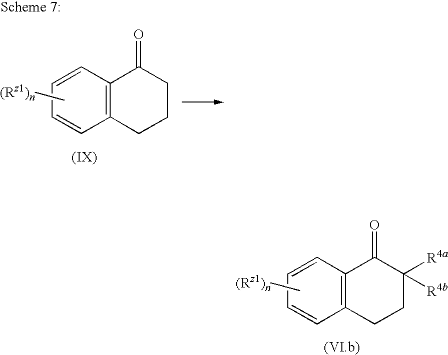

For instance ketones (VI) wherein at least one of the radicals R^{4a} or R^{4b} is different from hydrogen and B is CH₂ (referred to as compounds (VI.b)) can be obtained from a suitable α-unsubstituted ketone (IX) via alkylation or palladium-catalyzed arylation (see M. Palucki, S. L. Buchwald J. Am. Chem. Soc. 1997, 119, 11108-11109 or J. M. Fox, X. Huang, A. Chieffi, S. L. Buchwald J. Am. Chem. Soc. 2000, 122, 1360-1370) of the enolate species.

Alternatively α-substituted ketones (VI) can be obtained from an intramolecular Friedel-Crafts-Acylation of a suitable aromatic acid chloride (see Y. Oshiro et al. J. Med. Chem. 1991, 34, 2004-2013 or W. Vaccaro et al. J. Med. Chem. 1996, 39, 1704-1719).

Compounds of the formula (I.a) and (I.b) wherein R^{2a} and R^{2b}, respectively, are different from hydrogen (referred to as compounds (I.A4) and (I.B4)), can be obtained as outlined in scheme 8.

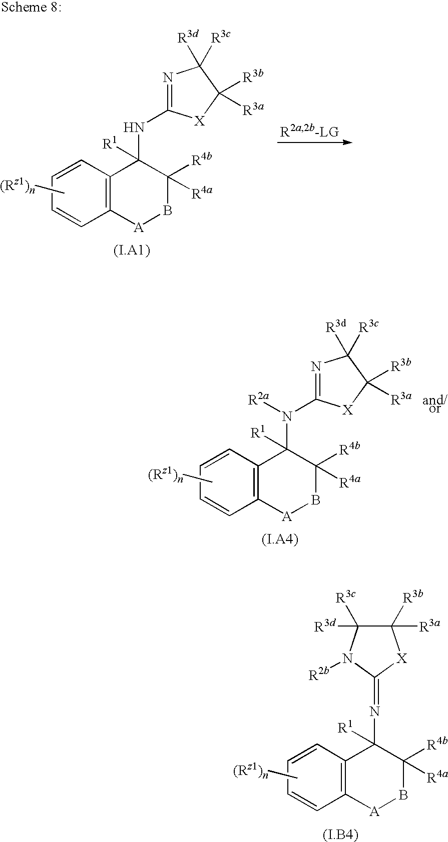

wherein X, (R^{z1})ₙ, A, B, R¹, R^{3a}, R^{3b}, R^{3c}, R^{3d}, R^{4a}, R^{4b} and R⁵ are as defined above.

A compound (I.A1) wherein R^{2a} and R^{2b}, respectively, are hydrogen is treated with a suitable electrophile. Suitable electrophiles are e.g. an alkylating or acylating agent R^{2a,2b}-LG (LG=leaving group) e. g. as described in WO 2005063724.

Compounds of the formulae (II) and (III), respectively, can be prepared as shown in schemes 9 and 10 below.

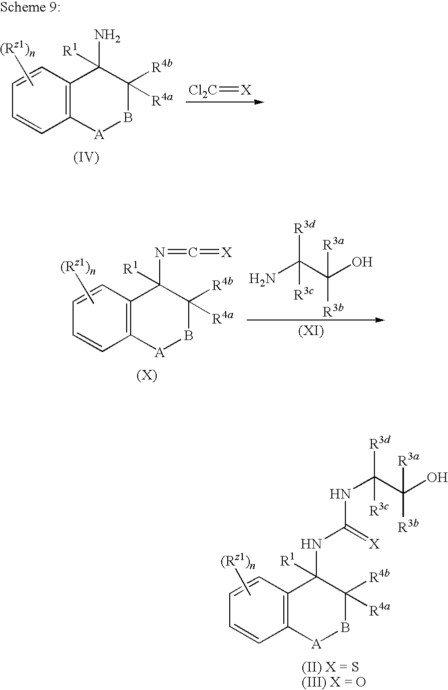

wherein (R^{z1})ₙ, A, B, R¹, R^{3a}, R^{3b}, R^{3c}, R^{3d}, R^{4a} and R^{4b} are as defined above.

An amine (IV) or a salt thereof is converted to the corresponding iso(thio)cyanate (VI) by conventional means, e.g. by reacting (IV) with (thio)phosgene, as described for example in the case of thiophosgene in Houben-Weyl, E4, “Methoden der Organischen Chemie”, chapter IIc, pp. 837-842, Georg Thieme Verlag 1983. It may be advantageous to carry out the reaction in the presence of a base. The iso(thio)cyanate (X) is then reacted with an aminoethanol (XI) to form an amino(thio)carbonylaminoethane compound. The reaction of the aminoethanol (XI) with iso(thio)cyanate (V) can be performed in accordance with standard methods of organic chemistry, see e.g. Biosci. Biotech. Biochem. 56 (7), 1062-65 (1992).

Another method for preparing compounds of formula (II), wherein X is S is shown in scheme 10.

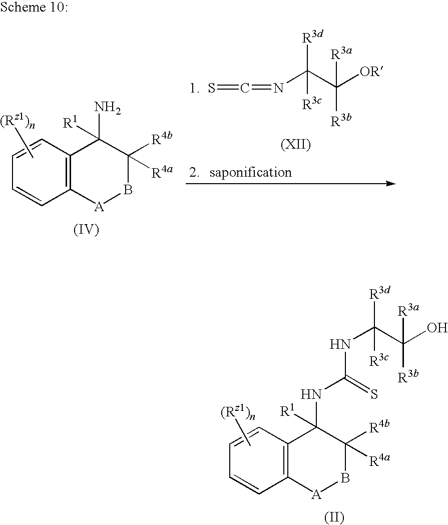

wherein (R^{z1})ₙ, A, B, R¹, R^{3a}, R^{3b}, R^{3c}, R^{3d}, R^{4a} and R^{4b} are as defined above and R′ is e.g. benzoyl.

An amine (IV) or a salt thereof can be converted to the corresponding thiourea compound (II), by reacting the amine (IV) with an isothiocyanate (XII) and subsequent saponification (see G. Liu et al. J. Org. Chem. 1999, 64, 1278-1284). Isothiocyanates of formula (XII) can be prepared according to the procedures described in Coll. Czech. Chem. Commun. 1986, 51, 112-117.

As a rule, the azoline compounds of the formulae (I.a) and (I.b) can be prepared by the methods described above. However, in individual cases, certain compounds (I.a) or (I.b) can also advantageously be prepared from other compounds I by ester hydrolysis, amidation, esterification, ether cleavage, olefination, reduction, oxidation and the like.

Due to their excellent activity, the compounds of the general formulae (I.a) and (I.b) may be used for controlling animal pests, selected from harmful insects, acarids and nematodes.

Accordingly, the invention further provides agriculturally composition for combating such animal pests, which comprises such an amount of at least one compound of the general formulae (I.a) and (I.b), respectively, or at least an agriculturally useful salt of (I.a) and (I.b), respectively, and at least one inert liquid and/or solid agronomically acceptable carrier that has a pesticidal action and, if desired, at least one surfactant.

Such a composition may contain a single active compound of the formulae (I.a) and (I.b), respectively, or the enantiomers thereof or a mixture of several active compounds (I.a) and compounds (I.b), respectively, according to the present invention. The composition according to the present invention may comprise an individual isomer or mixtures of isomers as well as individual tautomers or mixtures of tautomers.

The compounds of the formula (I.a) as well as the compounds of the formula (I.b) and the pestidicidal compositions comprising them are effective agents for controlling arthropod pests and nematodes. Animal pests controlled by the compounds of formula (I.a) include for example

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria;

dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa;

thrips (Thysanoptera), e.g. Dichromothrips corbetti, Frankliniella fusca, Frankliniella occidentalis, Frankliniella tritici, Scirtothrips citri, Thrips oryzae, Thrips palmi and Thrips tabaci;

hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta;

heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor;

homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus persicae, Myzus varians, Nasonovia ribis-nigri, Nilaparvata lugens, Pemphigus bursarius, Perkinsiella saccharicida, Phorodon humuli, Psylla mali, Psylla pin, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Sogatella furcifera Trialeurodes vaporariorum, Toxoptera aurantiiand, and Viteus vitifolii;

termites (Isoptera), e.g. Calotermes flavicollis, Leucotermes flavipes, Reticulitermes flavipes, Reticulitermes lucifugus and Termes natalensis;

orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;

Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;

Siphonatera, e.g. Xenopsylla cheopsis, Ceratophyllus spp;

The compositions and compounds of formula (I.a) as well as the compositions and compounds of formula (I.b) are useful for the control of nematodes, especially plant parasitic nematodes such as root knot nematodes, Meloidogyne hapla, Meloidogyne incognita, Meloidogyne javanica, and other Meloidogyne species;

cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

In a preferred embodiment of the invention the compounds of formula (I.a) as well as the compounds of formula (I.b) are used for controlling insects or arachnids, in particular insects of the orders Lepidoptera, Coleoptera and Homoptera and arachnids of the order Acarina. The compounds of the formula (I.a) according to the present invention are particularly useful for controlling insects of the order Thysanoptera and Homoptera.

The compounds of formula (I.a) as well as the compounds of formula (I.b) or the pesticidal compositions comprising them may be used to protect growing plants and crops from attack or infestation by animal pests, especially insects, acaridae or arachnids by contacting the plant/crop with a pesticidally effective amount of compounds of formula (I.a) and formula (I.b), respectively. The term “crop” refers both to growing and harvested crops.

The compounds of formula (I.a) as well as the compounds of formula (I.b), can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4^{th} Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and/or binders and/or gelling agents.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (N-methyl-pyrrolidones [NMP], N-octyl-pyrrolidone [NOP]), acetates (glycol diacetate), glycols, fatty acid dimethylamides, fatty acids and fatty acid esters. In principle, solvent mixtures may also be used.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

A suitable preservative is e.g. dichlorophen.

Seed treatment formulations may additionally comprise binders and optionally colorants.

Binders can be added to improve the adhesion of the active materials on the seeds after treatment. Suitable binders are block copolymers EO/PO surfactants but also polyvinylalcoholsl, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®).

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers.

Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compounds of formula (I.a) as well as the compounds of formula (I.b) can be used as such, in the form of their formulations or the use forms prepared therefrom, for example in the form of directly sprayable solutions, powders, sus-pensions or dispersions, emulsions, oil dispersions, pastes, dustable products, materi-als for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations:
1. Products for dilution with water for foliar applications. For seed treatment purposes, such products may be applied to the seed diluted or undiluted.

A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formulation with 10% (w/w) of active compound(s) is obtained.

B) Dispersible Concentrates (DC)

20 parts by weight of the active compound(s) are dissolved in 70 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

C) Emulsifiable Concentrates (EC)

15 parts by weight of the active compound(s) are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

D) Emulsions (EW, EO, ES)

25 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

H) Gel-Formulation (GF)

In an agitated ball mill, 20 parts by weight of the active compound(s) are comminuted with addition of 10 parts by weight of dispersants, 1 part by weight of a gelling agent wetters and 70 parts by weight of water or of an organic solvent to give a fine active compound(s) suspension. Dilution with water gives a stable suspension of the active compound(s), whereby a formulation with 20% (w/w) of active compound(s) is obtained.
2. Products to be applied undiluted for foliar applications. For seed treatment purposes, such products may be applied to the seed diluted or undiluted.

I) Dustable Powders (DP, DS)

5 parts by weight of the active compound(s) are ground finely and mixed intimately with 95 parts by weight of finely divided kaolin. This gives a dustable product having 5% (w/w) of active compound(s)

J) Granules (GR, FG, GG, MG)

0.5 parts by weight of the active compound(s) is ground finely and associated with 95.5 parts by weightof carriers, whereby a formulation with 0.5% (w/w) of active compound(s) is obtained. Current methods are extrusion, spray-drying or the fluidized bed. This gives granules to be applied undiluted for foliar use.

K) ULV Solutions (UL)

10 parts by weight of the active compound(s) are dissolved in 90 parts by weight of an organic solvent, for example xylene. This gives a product having 10% (w/w) of active compound(s), which is applied undiluted for foliar use.

The compounds of formula (I.a) as well as the compounds of formula (I.b) are also suitable for the treatment of seeds. Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typcially, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Other preferred FS formulations of compounds of formula (I.a) for seed treatment comprise from 0.5 to 80 wt % of the active ingredient, from 0,05 to 5 wt % of a wetter, from 0.5 to 15 wt % of a dispersing agent, from 0,1 to 5 wt % of a thickener, from 5 to 20 wt % of an anti-freeze agent, from 0,1 to 2 wt % of an anti-foam agent, from 1 to 20 wt % of a pigment and/or a dye, from 0 to 15 wt % of a sticker/adhesion agent, from 0 to 75 wt % of a filler/vehicle, and from 0,01 to 1 wt % of a preservative.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula (I.a) as well as the compounds of formula (I.b) are effective through both contact (via soil, glass, wall, bed net, carpet, plant parts or animal parts), and ingestion (bait, or plant part).

For use against ants, termites, wasps, flies, mosquitos, crickets, or cockroaches, compounds of formula (I.a) as well as compounds of formula (I.b) are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula (I.a) as well as formulations of compounds of formula (I.b) as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylfomaamide, N-methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula (I.a) as well as the compounds of formula (I.b) and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula (I.a) as well as compounds of formula (I.b) and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methyl-neodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula (I.a) as well as the compounds of formula (I.b) or the enantiomers or veterinarily acceptable salts thereof are in particular also suitable for being used for combating parasites in and on animals.

An object of the present invention is therfore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula (I.a) and formula (I.b), a respectively, or the enantiomers or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula (I.a) and formula (I.b), respectively, or the enantiomers or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula (I.a) and formula (I.b), respectively, or the enantiomers or veterinarily acceptable salts thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly, it has now been found that compounds of formula (I.a) as well as compounds of formula (I.b) are suitable for combating endo- and ectoparasites in and on animals.

Compounds of formula (I.a) as well as compounds of formula (I.b) or the enantiomers or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula (I.a) as well as compounds of formula (I.b) or the enantiomers or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula (I.a) as well as compounds of formula (I.b) or the enantiomers or veterinarily acceptable salts thereof and compositions comprising them are suitable for systemic and/or non-systemic control of ecto- and/or endoparasites. They are active against all or some stages of development.

The compounds of formula (I.a) as well as compounds of formula (I.b) are especially useful for combating ectoparasites.

The compounds of formula (I.a) as well as compounds of formula (I.b) are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,

Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,

Bugs (Heteropterida): Cimex lectularius, Cimex hemipterus, Reduvius senilis, Triatoma spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

Roundworms Nematoda:

Wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Helicephalobus spp,

Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,

Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,

Thorny headed worms (Acanthocephala), e.g. Acanthocephalus spp., Macracanthorhynchus hirudinaceus and Oncicola spp,

Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula (I.a) as well as compounds of formula (I.b) and compositions containing them are particularly useful for the control of pests from the orders Diptera, Siphonaptera and Ixodida.

Moreover, the use of the compounds of formula (I.a) as well as compounds of formula (I.b) and compositions containing them for combating mosquitoes is especially preferred.

The use of the compounds of formula (I.a) as well as compounds of formula (I.b) and compositions containing them for combating flies is a further preferred embodiment of the present invention.

Furthermore, the use of the compounds of formula (I.a) as well as compounds of formula (I.b) and compositions containing them for combating fleas is especially preferred.

The use of the compounds of formula (I.a) as well as the compounds of formula (I.b) and compositions containing them for combating ticks is a further preferred embodiment of the present invention.

The compounds of formula (I.a) as well as the compounds of formula (I.b) also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula (I.a) compounds as well as the formula (I.b) compounds may be formulated as animal feeds, animal feed premixes, animal feed concentrates, pills, solutions, pastes, suspensions, drenches, gels, tablets, boluses and capsules. In addition, the formula (I.a) compounds may be administered to the animals in their drinking water. For oral administration, the dosage form chosen should provide the animal with 0.01 mg/kg to 100 mg/kg of animal body weight per day of the formula (I.a) compound, preferably with 0.5 mg/kg to 100 mg/kg of animal body weight per day.

Alternatively, the formula (I.a) compounds as well as formula (I.b) compounds may be administered to animals parenterally, for example, by intraruminal, intramuscular, intravenous or subcutaneous injection. The formula (I.a) compounds as well as formula (I.b) compounds may be dispersed or dissolved in a physiologically acceptable carrier for subcutaneous injection. Alternatively, the formula (I.a) compounds as well as formula (I.b) compounds may be formulated into an implant for subcutaneous administration. In addition the formula (I.a) compound as well as formula (I.b) compounds may be transdermally administered to animals. For parenteral administration, the dosage form chosen should provide the animal with 0.01 mg/kg to 100 mg/kg of animal body weight per day of the formula (I.a) compound.

The formula (I.a) compounds as well as formula (I.b) compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula (I.a) compound. In addition, the formula (I.a) compounds as well as formula (I.b) compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents are water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C_{18,} isopropyl myristate, isopropyl palmitate, caprylic/capric acid esters of saturated fatty alcohols of chain length C₁₂-C_{18,} isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter,

fatty alcohols such as isotridecyl alcohol, 2-octyldodecanol, cetylstearyl alcohol, ( )eyl alcohol, and

fatty acids such as oleic acid and

mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether;

ampholytic surfactants such as di-sodium N-lauryl-p-iminodipropionate or lecithin;

anionic surfactants, such as sodium lauryl sulfate, fatty alcohol ether sulfates, mono/dialkyl polyglycol ether orthophosphoric acid ester monoethanolamine salt;

cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compound of formulae (I.a) or (I.b), respectively.

Generally, it is favorable to apply the compounds of formula (I.a) as well as compounds of formula (I.b) in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula (I.a) and formula (I.b), respectively, against endoparasites in concentrations of 10 ppm to 2 per cent by weight, preferably of 0.05 to 0.9 percent by weight, very particularly preferably of 0.005 to 0.25 percent by weight.

In a preferred embodiment of the present invention, the compositions comprising the compounds of formula (I.a) and the compound of formula (I.b), respectively, are applied dermally/topically.

In a further preferred embodiment, the topical application is conducted in the form of compound-containing shaped articles such as collars, medallions, ear tags, bands for fixing at body parts, and adhesive strips and foils.

Generally, it is favorable to apply solid formulations which release compounds of formula (I.a) and formula (I.b), respectively, in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula (I.a) and formula (I.b). A detailed list of plastics and elastomers as well as preparation procedures for the shaped articles is given e.g. in WO 03/086075.

Compositions to be used according to this invention may also contain other active ingredients, for example other pesticides, insecticides, herbicides, fungicides, other pesticides, or bactericides, fertilizers such as ammonium nitrate, urea, potash, and super-phosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents can be admixed with the agents used according to the invention in a weight ratio of 1:10 to 10:1. Mixing the compounds (I.a) and the compounds (I.b), respectively, or the compositions comprising them in the use form as pesticides with other pesticides frequently results in a broader pesticidal spectrum of action.

The following list of pesticides together with which the compounds of formula (I.a) and the compounds of the formula (I.b), respectively, can be used, is intended to illustrate the possible combinations, but not to impose any limitation:

A.1. Organo(thio)phosphates: acephate, azamethiphos, azinphos-ethyl, azinphosmethyl, chlorethoxyfos, chlorfenvinphos, chlormephos, chlorpyrifos, chlorpyrifosmethyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;

A.2. Carbamates: aldicarb, alanycarb, bendiocarb, benfuracarb, butocarboxim, butoxycarboxim, carbaryl, carbofuran, carbosulfan, ethiofencarb, fenobucarb, formetanat, furathiocarb, isoprocarb, methiocarb, methomyl, metolcarb, oxamyl, pirimicarb, propoxur, thiodicarb, thiofanox, trimethacarb, XMC, xylylcarb, triazamate;

A.3. Pyrethroids: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cyphenothrin, cypermethrin, alpha-cypermethrin, beta-cypermethrin, theta-cypermethrin, zeta-cypermethrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, resmethrin, RU 15525, silafluofen, tefluthrin, tetramethrin, tralomethrin, transfluthrin, ZXI 8901;

A.4. Juvenile hormone mimics: hydroprene, kinoprene, methoprene, fenoxycarb, pyriproxyfen;

A.5. Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), thiacloprid, thiocyclam, thiosultap-sodium, and AKD 1022.

A.6. GABA gated chloride channel antagonist compounds: chlordane, endosulfan, gamma-HCH (lindane); acetoprole, ethiprole, fipronil, pyrafluprole, pyriprole, vaniliprole, the phenylpyrazole compound of formula (Γ¹)

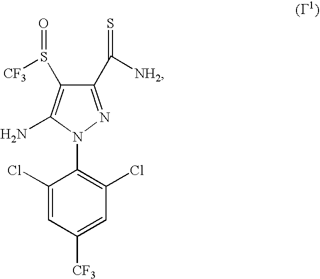

A.7. Chloride channel activators: abamectin, emamectin benzoate, milbemectin, lepimectin;

A.8. METI I compounds: fenazaquin, fenpyroximate, pyrimidifen, pyridaben, tebufenpyrad, tolfenpyrad, flufenerim, rotenone;

A.9. METI II and III compounds: acequinocyl, fluacyprim, hydramethylnon;

A.10. Uncouplers of oxidative phosphorylation: chlorfenapyr, DNOC;

A.11. Inhibitors of oxidative phosphorylation: azocyclotin, cyhexatin, diafenthiuron, fenbutatin oxide, propargite, tetradifon;

A.12. Moulting disruptors: cyromazine, chromafenozide, halofenozide, methoxyfenozide, tebufenozide;

A.13. Synergists: piperonyl butoxide, tribufos;

A.14. Sodium channel blocker compounds: indoxacarb, metaflumizone;

A.15. Fumigants: methyl bromide, chloropicrin sulfuryl fluoride;

A.16. Selective feeding blockers: crylotie, pymetrozine, flonicamid;

A.17. Mite growth inhibitors: clofentezine, hexythiazox, etoxazole;

A.18. Chitin synthesis inhibitors: buprofezin, bistrifluron, chlorfluazuron, diflubenzuron, flucycloxuron, flufenoxuron, hexaflumuron, lufenuron, novaluron, noviflumuron, teflubenzuron, triflumuron;

A.19. Lipid biosynthesis inhibitors: spirodiclofen, spiromesifen, spirotetramat;

A.20. octapaminergic agonsits: amitraz;

A.21. ryanodine receptor modulators: flubendiamide;

A.22. Various: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, tartar emetic;

A.23. N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl;

A.24. Anthranilamides: chloranthraniliprole, the compound of formula Γ²

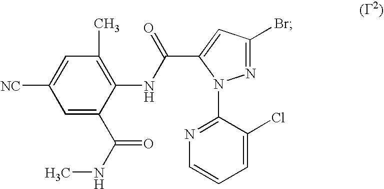

A.25. Malononitrile compounds: CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₂)₂CF₂H, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, CF₃CF₂CH₂C(CN)₂CH₂(CF₂)₃CF₂H, 2-(2,2,3,3,4,4,5,5-octafluoropentyl)-2-(3,3,4,4,4-pentafluorobutyl)-malonodinitrile, and CF₂HCF₂CF₂CF₂CH₂C(CN)₂CH₂CH₂CF₂CF₃;

A.26. Microbial disruptors: Bacillus thuringiensis subsp. lsraelensi, Bacillus sphaericus, Bacillus thuringiensis subsp. Aizawai, Bacillus thuringiensis subsp. Kurstaki, Bacillus thuringiensis subsp. Tenebrionis;

A.27. Alkynylether compounds Γ⁴ and Γ⁵:

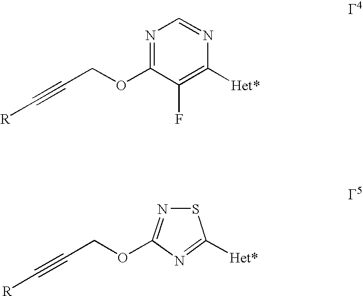

wherein R is methyl or ethyl and Het* is 3,3-dimethylpyrrolidin-1-yl, 3-methylpiperidin-1-yl, 3,5-dimethylpiperidin-1-yl, 3-trifluormethylpiperidin-1-yl, hexahydroazepin-1-yl, 2,6-dimethylhexahydroazepin-1-yl or 2,6-dimethylmorpholin-4-yl. These compounds are described e.g. in JP 2006131529.

The commercially available compounds of the group A may be found in The Pesticide Manual, 13^{th} Edition, British Crop Protection Council (2003) among other publications. Thioamides of formula Γ¹ and their preparation have been described in WO 98/28279. Lepimectin is known from Agro Project, PJB Publications Ltd, November 2004. Benclothiaz and its preparation have been described in EP-Al 454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1 462 456. Flupyrazofos has been described in Pesticide Science 54, 1988, p. 237-243 and in U.S. Pat No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. AKD 1022 and its preparation have been described in U.S. Pat. No. 6,300,348. Chloranthraniliprole has been described in WO 01/70671, WO 03/015519 and WO 05/118552. Anthranilamide derivatives of formula Γ² have been described in WO 01/70671, WO 04/067528 and WO 05/118552. Cyflumetofen and its preparation have been described in WO 04/080180. The aminoquinazolinone compound pyrifluquinazon has been described in EP A 109 7932. The malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, CF₃CF₂CH₂C(CN)₂CH₂(CF₂)₃CF₂H, 2-(2,2,3,3,4,4,5,5-octafluoropentyl)-2-(3,3,4,4,4-pentafluorobutyl)-malonodinitrile, and CF₂HCF₂CF₂CF₂CH₂C(CN)₂CH₂CH₂CF₂CF₃ have been described in WO 05/63694.

Fungicidal mixing partners are those selected from the group consisting of

acylalanines such as benalaxyl, metalaxyl, ofurace, oxadixyl,

amine derivatives such as aldimorph, dodine, dodemorph, fenpropimorph, fenpropidin, guazatine, iminoctadine, spiroxamin, tridemorph,

anilinopyrimidines such as pyrimethanil, mepanipyrim or cyrodinyl,

antibiotics such as cycloheximid, griseofulvin, kasugamycin, natamycin, polyoxin or streptomycin,

azoles such as bitertanol, bromoconazole, cyproconazole, difenoconazole, diniconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol,

dicarboximides such as iprodion, myclozolin, procymidon, vinclozolin,

dithiocarbamates such as ferbam, nabam, maneb, mancozeb, metam, metiram, propineb, polycarbamate, thiram, ziram, zineb,

heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine,

copper fungicides such as Bordeaux mixture, copper acetate, copper oxychloride, basic copper sulfate,

nitrophenyl derivatives such as binapacryl, dinocap, dinobuton, nitrophthalisopropyl, phenylpyrroles such as fenpiclonil or fludioxonil,

sulfur,

other fungicides such as acibenzolar-S-methyl, benthiavalicarb, carpropamid, chlorothalonil, cyflufenamid, cymoxanil, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid,

strobilunns such as azoxystrobin, dimoxystrobin, fluoxastrobin, kresoxim-methyl, metominostrobin, orysastrobin, picoxystrobin or trifloxystrobin,

sulfenic acid derivatives such as captafol, captan, dichlofluanid, folpet, tolylfluanid, cinnemamides and analogs such as dimethomorph, flumetover or flumorph.

The animal pest, i.e. arthropodes and nematodes, the plant, soil or water in which the plant is growing can be contacted with the present compound(s) (I.a) as well as compound(s) (I.b) or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

Moreover, animal pests may be controlled by contacting the target pest, its food supply, habitat, breeding ground or its locus with a pesticidally effective amount of compounds of formula (I.a) or (I.b). As such, the application may be carried out before or after the infection of the locus, growing crops, or harvested crops by the pest.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula (I.a) and its compositions as well as compounds of formula (I.b) and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula (I.a) as well as the compounds of formula (I.b) are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of the invention can also be applied preventively to places at which occurrence of the pests is expected.

The compounds of formula (I.a) as well as compounds of formula (I.b) may be also used to protect growing plants from attack or infestation by pests by contacting the plant with a pesticidally effective amount of compounds of formulae (I.a) or (I.b). As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95% by weight, preferably from 0.1 to 45% by weight, and more preferably from 1 to 25% by weight of at least one repellent and/or insecticide.

For use in bait compositions, the typical content of active ingredient is from 0.001% by weight to 15% by weight, desirably from 0.001% by weight to 5% by weight of active compound.

For use in spray compositions, the content of active ingredient is from 0.001 to 80% by weight, preferably from 0.01 to 50% by weight and most preferably from 0.01 to 15% by weight.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the mixture are generally from 0.1 g to 10 kg per 100 kg of seed, preferably from 1 g to 5 kg per 100 kg of seed, in particular from 1 g to 200 g per 100 kg of seed.

The present invention is now illustrated in further detail by the following examples.

### 1. SYNTHETIC EXAMPLES

### 1.1. Preparation of (4,5-Dihydro-thiazol-2-yl)-(2,2-dimethyl-indan-1-yl)-amine

1.1.1. Indan-1-one (2.00 g, 15.13 mmol) was added to a suspension of sodium hydride (60%, 1.33 g, 33.29 mmol) in DMF (20 mL). After 1 h methyl iodide (15.04 g, 105.93 mmol) was added and the reaction mixture was stirred for 1.5 h. Addition of water, extraction with diethyl ether (2 times), washing the combined organic layers with water (4 times) and drying over Na₂SO₄ yielded the crude product 2,2-Dimethyl-indan-1-one (2.40 g, 14.98 mmol, 99%) which was used without any further purification.

1.1.2. Molecular sieves (3 Å, 4.00 g), ammonium acetate (4.81 g, 62.42 mmol) and 2,2-Dimethyl-indan-1-one (1.00 g, 6.24 mmol) were suspended in ethanol (40 mL) and stirred for 1.5 h at 30° C. Sodium cyanoborhydride (1.18 g, 18.72 mmol) was added at room temperature and the reaction mixture heated to 80° C. for 21 h. After cooling to room temperature the mixture was filtered through Celite, the residue was washed with ethanol (2 times) and the solvent removed in vacuo. The residue was taken up in diethyl ether, brought to alkaline pH with aq. ammonia and extracted with ether (3 times). The combined organic layers were treated with ethereal HCl (1 M) to precipitate the hydrochloride salt of 2,2-dimethyl-indan-1-ylamine (0.70 g, 3.54 mmol, 57%). Extraction with an aq. solution of NaOH (1 M) and dichloromethane yielded the free base.

1.1.3. 2,2-Dimethyl-indan-1-ylamine (410 mg, 2.54 mmol) was dissolved in diethylether (15 mL) and cooled to -10° C. 1-chloro-2-isothiocyanatoethane (310 mg, 2.54 mmol) was added dropwise and the reaction mixture was stirred over night at a temperature in a range from −10 to 0° C. After addition of water (10 mL) and an aqueous solution of NaOH (1 M, 5 mL), the layers were separated and the aqueous layer was extracted two times with diethylether. The combined organic layers were washed with water and dried over Na₂SO₄. The crude product was purified by column chromatography (SiO₂, CH₂Cl₂/MeOH, gradient 100:0 to 90:10) to yield (4,5-dihydro-thiazol-2-yl)-(2,2-dimethyl-indan-1-yl)-amine (280 mg, 1.14 mmol, 45%) and (2,2-dimethyl-indan-1-yl)(4,5,4′,5′-tetrahydro-[2,3]bithiazolyl-2′-ylidene) amine (160 mg, 0.48 mmol, 19%).

### 1.2. Preparation of 3-(3,5-Dichloro-phenyl)-indan-1-ylamine

1.2.1. 1-(2-Bromo-phenyl)-3-(3,5-dichloro-phenyl)-propenone (4.50 g, 12.60 mmol), N.N-dimethylcyclohexyl amine (3.22 g, 25.28 mmol) and Pd(PPh)₃Cl₂ (0.18 g, 0.25 mmol) were dissolved in DMF (25 mL). The reaction mixture was heated in a microwave oven (200 W, Tₘₐₓ=125° C., pₘₐₓ=12 bar) for 25 min. After filtration and evaporation of the volatiles the crude product was purified by column chromatographie (SiO₂, cyclohexane/ethyl acetate, gradient 100:0 to 85:15) to yield 3-(3,5-Dichloro-phenyl)-indan-1-one (2.00 g, 7.20 mmol, 57%).

1.2.2. 3-(3,5-Dichloro-phenyl)-indan-1-one (1.19 g, 4.30 mmol), potassium carbonate (1.19 g, 8.60 mmol) and hydroxyl amine hydro chloride (0.60 g, 8.60 mmol) were dissolved in ethanol (25 mL) and heated to reflux for 2 h. After addition of water (60 mL) the precipitate was filtered, washed with water and dried to yield 3-(3,5-Dichloro-phenyl)-indan-1-one oxime (1.15 g, 3.94 mmol, 92%).

1.2.3. 3-(3,5-Dichloro-phenyl)-indan-1-one oxime (1.00 g, 3.42 mmol) and Raney-Nickel (1.50 g) were suspended in MeOH (30 mL) and hydrogenated at 5 bar for 12 h. Filtration and evaporation of the volatiles gave a crude product which was purified by column chromatographic purification (SiO₂, MeOH/CH₂Cl₂, gradient 100:0 to 90:10) to yield 3-(3,5-Dichloro-phenyl)-indan-1-ylamine (520 mg (350 mg cis, 80 mg trans, 90 mg cis/trans 1:1), 1.87 mmol, 55%).

Compounds of the formula (I.a) (wherein R¹, R^{2a}, R^{3a}, R^{3b}, R^{3c} and R^{3d} are hydrogen and X is sulfur) listed in table I below were prepared in an analogous manner.

TABLE I

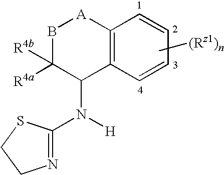

| Ex. | | | | | Physico-chemical data |
|---|---|---|---|---|---|
| no. | (R^{z1})ₙ | A | B | R^{4a}, R^{4b} | m.p. [° C.]; r.t.; M⁺; (*) |
| 1a | | CH(3,5-di- | — | H, H | n.d.; 2.63; 323 |
| | | methylphenyl) | | | |
| 1b | | CH(3,5-di- | — | H, H | n.d.; 2.97; 323 |
| | | methylphenyl) | | | (trans) |
| 1c | | CH(3,5-di- | — | H, H | 90-95; n.d.; n.d. |
| | | methylphenyl) | | | (cis) |
| 2a | | CH(3,5-di- | — | H, H | n.d.; 2.64, 355 |
| | | methoxyphenyl) | | | |
| 2b | | CH(3,5-di- | — | H, H | n.d.; 2.66; 355 |
| | | methoxyphenyl) | | | (cis) |
| 2c | | CH(3,5-di- | — | H, H | n.d.; 2.61; 355 |
| | | methoxyphenyl) | | | (trans) |
| 3a | | CH(3,5-di- | — | H, H | n.d.; 3.10; 364 |
| | | chlorophenyl) | | | |
| 3b | | CH(3,5-di- | — | H, H | 115-121; n.d.; n.d. |
| | | chlorophenyl) | | | (cis) |
| 3c | | CH(3,5-di- | — | H, H | n.d.; 3.09; 364 |
| | | chlorophenyl) | | | (trans) |
| 4a | | CH(phenyl) | — | H, H | 125-130; n.d.; n.d. |
| | | | | | (cis) |
| 4b | | CH(phenyl) | — | H, H | n.d.; 2.61; 295; |
| | | | | | (trans) |
| 5 | | CH(3,5-di- | — | H, H | n.d.; 2.72; 331 |
| | | fluorophenyl) | | | |
| 6a | | CH(benzyl) | — | H, H | 135-139; n.d.; n.d. |
| | | | | | (cis) |
| 6b | | CH(benzyl) | — | H, H | n.d.; 2.75; 309 |
| 6c | | CH(benzyl) | — | H, H | n.d.; 2.72; 309 |
| | | | | | (trans) |
| 7 | 1- | O | — | H, H | 134-136; n.d.; n.d. |
| | OCH₃ | | | | |
| 8 | | O | — | H, H | 135-136; n.d.; n.d. |
| 9 | 1-F, | O | — | H, H | 130-131; n.d.; n.d. |
| | 3-F | | | | |
| 10 | 3- | O | — | H, H | 91-93; n.d.; n.d. |
| | OCH₃ | | | | |
| 11 | 1-Cl, | O | — | H, H | 140-144; n.d.; n.d. |
| | 3-Cl | | | | |
| 12 | 3-F | O | — | H, H | 103-105; n.d.; n.d. |
| 13 | 3-Br | O | — | H, H | n.d.; 2.03; 299 |
| 14 | 3-Cl | O | — | H, H | 136-139; n.d.; n.d. |
| 15 | | S | — | H, H | 141-143; n.d.; n.d. |
| 16 | 1-F | O | — | H, H | 134-136; n.d.; n.d. |
| 17 | 1-Cl | O | — | H, H | 147-149; n.d.; n.d. |
| 18 | | S(O)₂ | — | H, H | n.d.; 1.16; 269 |
| 19a | — | CH₂ | — | CH₃, H | 109-112; n.d.; n.d. |
| 19b | — | CH₂ | — | CH₃, H | 125-126; n.d.; n.d. |
| | | | | | (trans) |
| 20 | — | CH₂ | — | C₂H₅, H | 123-125; n.d.; n.d. |
| 21a | — | CH₂ | — | F, H | 158-161; n.d.; n.d. |
| 21b | — | CH₂ | — | F, H | n.d.; 1.88-237 |
| 22 | — | CH₂ | — | CH₃, CH₃ | 117-119; n.d.; n.d. |
| 23 | — | CH₂ | — | n-prop-2-enyl, H | n.d.; 2.50; 259 |
| 24 | — | CH₂ | — | isopent-2-enyl, H | 79-81; n.d.; n.d. |
| 25 | — | CH₂ | — | F, F | 147-149; n.d.; n.d. |
| 26 | — | CH₂ | — | prop-3-ynyl, H | n.d.; 2.25; 257 |
| 27 | — | CH₂ | — | 3,5-dimethylbenzyl, H | 124-125; n.d.; n.d. |
| 28 | — | O | CH₂ | H, H | 190-193; n.d.; n.d. |
| 29 | — | S | CH₂ | H, H | 158-163; n.d.; n.d. |

Ex. = example
m.p. = melting point
r.t. = retention time (HPLC)
M⁼ = molecular mass of the detected cationic species
(*) = relativ configuration of the amino group towards another substituent (i.e. R^{4a}, R^{4b}, R^{6a} or R^{6b}) in the isolated diastereomere
n.d. = n.d.

Compounds of the formula (I.b) (wherein R¹, R^{3a}, R^{3b}, R^{3c} and R^{3d} are hydrogen and X is sulfur) listed in table II below were prepared in an analogous manner.

TABLE II

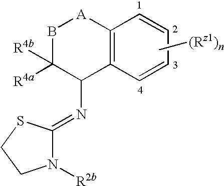

| Ex. | | | | | | Physico-chemical data |
|---|---|---|---|---|---|---|
| no. | (R^{z1})ₙ | A | B | R^{2b} | R^{4a}, R^{4b} | m.p. [° C.]; r.t.; M⁺; (*) |
| 30a | | CH(3,5-di | — | 2-thiazolyl | H, H | n.d.; 2.99; 408 |
| | | methylphenyl) | | | | |
| 30b | | CH(3,5-di- | — | 2-thiazolyl | H, H | 165-170; n.d.; n.d. |
| | | methylphenyl) | | | | (cis) |
| 30c | | CH(3,5-di- | — | 2-thiazolyl | H, H | 201-203; 3.43; 408 |
| | | methylphenyl) | | | | (trans) |
| 31a | | CH(3,5-di- | — | 2-thiazolyl | H, H | n.d.; 3.08; 440 |
| | | methoxyphenyl) | | | | |
| 31b | | CH(3,5-di- | — | 2-thiazolyl | H, H | n.d.; 3.09; 440 |
| | | methoxyphenyl) | | | | |
| 31c | | CH(3,5-di- | — | 2-thiazolyl | H, H | n.d.; 3.03; 440 |
| | | methoxyphenyl) | | | | |
| 32a | | CH(3,5-di- | — | 2-thiazolyl | H, H | n.d.; 3.57; 449 |
| | | chlorophenyl) | | | | |
| 32b | | CH(3,5-di- | — | 2-thiazolyl | H, H | 175-180; n.d.; n.d. |
| | | chlorophenyl) | | | | (cis) |
| 32c | | CH(3,5-di- | — | 2-thiazolyl | H, H | 225-230; n.d.; n.d. |
| | | chlorophenyl) | | | | (trans) |
| 33a | | CH(phenyl) | — | 2-thiazolyl | H, H | 73-76; n.d.; n.d. |
| | | | | | | (trans) |
| 33b | | CH(phenyl) | — | 2-thiazolyl | H, H | n.d.; 3.10; 380 |
| | | | | | | (trans) |
| 34 | | CH(3,5-di- | — | 2-thiazolyl | H, H | 161-163; n.d.; n.d. |
| | | fluorophenyl) | | | | (cis) |
| 35a | | CH(benzyl) | — | 2-thiazolyl | H, H | 152-158; n.d.; n.d. |
| | | | | | | (cis) |
| 35b | | CH(benzyl) | — | 2-thiazolyl | H, H | n.d.; 3.21; 394 |
| 35c | | CH(benzyl) | — | 2-thiazolyl | H, H | n.d.; 3.17; 3.94 |
| | | | | | | (trans) |
| 36 | 1- | O | — | 2-thiazolyl | H, H | 158-159; n.d.; n.d. |
| | OMe | | | | | |
| 37 | 1-F, | O | — | 2-thiazolyl | H, H | n.d.; 2.57-342 |
| | 3,F | | | | | |
| 38 | 3-F | O | — | 2-thiazolyl | H, H | n.d.; 2.26; 324 |
| 39 | 3-Cl | O | — | 2-thiazolyl | H, H | |
| 40 | | S | — | 2-thiazolyl | H, H | 134-137; n.d.; n.d. |
| 41 | | S(O)₂ | — | 2-thiazolyl | H, H | n.d.; 1.71; 354 |
| 42 | 1-F | O | — | 2-thiazolyl | H, H | 162-165; n.d.; n.d. |
| 43 | 1-Cl | O | — | 2-thiazolyl | H, H | 163-166; n.d.; n.d. |
| 44 | | CH₂ | — | 2-thiazolyl | C₂H₅, H | 148-151; n.d.; n.d. |
| 45 | | CH₂ | — | 2-thiazolyl | CH₃, H | 163-165; n.d.; n.d. |
| 46 | | CH₂ | — | 2-thiazolyl | F, H | n.d.; 2.29; 322 |
| 47 | | CH₂ | — | 2-thiazolyl | CH₃, CH₃ | 161-163; n.d.; n.d. |
| 48 | | CH₂ | — | 2-thiazolyl | n-prop-2-enyl, H | 126-129; n.d.; n.d. |
| 49 | | CH₂ | — | 2-thiazolyl | isopent-2-enyl, H | n.d.; 3.27; 372 |
| 50 | | CH₂ | — | 2-thiazolyl | F, F | 146-150; n.d.; n.d. |
| 51 | | CH₂ | — | 2-thiazolyl | prop-3-ynyl, H | 142-144; n.d.; n.d. |
| 52 | | CH₂ | — | 2-thiazolyl | 3,5-dimethyl- | 162-163; n.d.; n.d. |
| | | | | | benzyl, H | |
| 53 | | O | — | 2-thiazolyl | | 125-129; n.d.; n.d. |

Ex. = example
m.p. = melting point
r.t. = retention time
M⁺ = molecular mass of the detected cationic species
(*) = relativ configuration of the amino group towards another substituent (i.e. R^{4a}, R^{4b}, R^{6a} or R^{6b}) in the isolated diastereomere
n.d. = not determined

### 2. BIOLOGICAL EXAMPLES

### 2.1 Examples of Action Against Pests

The action of the compounds of the general formualae (I.a) and (I.b) against pests was demonstrated by the following experiments:

I. Cotton Aphid (Aphis gossypii)

Cotton plants in the cotyledon stage (variety ‘Delta Pine’) are infested with approximately 100 laboratory-reared aphids by placing infested leaf sections on top of the test plants. The leaf sections are removed after 24 hours. The cotyledons of the intact plants are dipped into gradient solutions of the test compound. Aphid mortality on the treated plants, relative to mortality on check plants, is determined after 5 days.

In this test, compound of example no. 5, 9, 12, 14, 15, 16, 19a, 19b, 20-23, 25, 26, 28, 46 and 50 at 300 ppm showed over 70% mortality in comparison with untreated controls.

I.b. Green Peach Aphid (Myzus persicae)

Pepper plants in the 2^{nd} leaf-pair stage (variety ‘California Wonder’) are infested with approximately 40 laboratory-reared aphids by placing infested leaf sections on top of the test plants. The leaf sections are removed after 24 hours. The leaves of the intact plants are dipped into gradient solutions of the test compound. Aphid mortality on the treated plants, relative to mortality on check plants, is determined after 5 days.

In this test, compound of example no. 8, 9, 11, 12, 15, 19a, 19b, 20-23, 25, 26, 28 and 46 at 300 ppm showed over 70% mortality in comparison with untreated controls.

1-28. (canceled)

29. A method for controlling an insect or a nematode pest, which comprises contacting the insect or nematode pest or its food supply, habitat, breeding ground or its locus with an agriculturally acceptable composition comprising a compound of the formula (I.a) or (I.b) and/or a salt thereof: 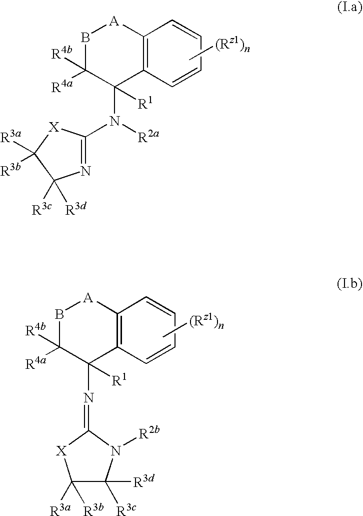
wherein
n is 0, 1, 2, 3 or 4.
X is sulfur, oxygen or a radical NR⁵;
A , is —C(R^{6a})(R^{6b})—, oxygen, NR⁷, sulfur, S(O) or S(O)₂;
B is a chemical bond or CH₂;
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl or benzyl, phenoxycarbonyl, 5 or 6 membered hetaryl and 5 or 6 membered hetarylmethyl each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl, a 5 or 6 membered heterocyclic ring and benzoyl each of the seven last mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members, and wherein the 5 or 6 membered heterocyclic ring contains 1, 2 or 3 heteroatoms selected from the group consisting of oxygen, sulfur and nitrogen as ring members; or
R¹ together with R^{2a} may be C₃-C₅-alkandiyl which may carry 1, 2, 3, 4 or 5 radicals R²¹ and which may be interrupted with 1 or 2 heteroatoms selected from the group consisting of oxygen, sulfur or nitrogen;
R¹ together with R^{2b} may also be a bridging carbonyl group C(O);
R^{3a}, R^{3b}, R^{3c}, R^{3d} are each independently selected from the group consisting of hydrogen, halogen, cyano, nitro, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, each of the last three mentioned radicals may be unsubstituted or may carry any combination of 1, 2, 3, 4 or 5 radicals R^{b3};
R^{4a}, R^{4b} are independently from each other selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
phenyl, benzyl, 5 or 6 membered hetaryl and 5 or 6 membered hetarylmethyl, each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O, ═NR^{c} or ═CR^{d}R^{e};
R^{2a} together with R^{4a} may form a bridging bivalent radical selected from the group consisting of C(O)—C(R^{24a})(R^{24b}), C(S)—C(R^{24a})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, S(O)—NH.
R⁵ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{6a}, R^{6b} are independently of each other selected from the group consisting of hydrogen, halogen, cyano, nitro, hydroxy, mercapto, amino, C₁-C₆-alkyl, C₁-C₆-haloalkyl, C₁-C₆-alkoxy, C₁-C₆-alkylamino, di-(C₁-C₆-alkyl)amino, wherein the carbon atoms in the last 5 mentioned radicals may be unsubstituted or may carry any combination of 1, 2 or 3 radicals R^{a6},
C₃-C₆-cycloalkyl, phenyl andbenzyl, each of the last three mentioned radicals may be unsubstituted or may carry any combination of 1, 2, 3, 4 or 5 radicals R^{b6}; or
R^{6a} together with R^{6b} may also be ═O, ═NR^{c} or ═CR^{d}R^{e};
R⁷ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a7},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b7}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{z1} are independently of each other selected from the group consisting of halogen, OH, SH, SO₃H, COOH, cyano, nitro, C₁-C₆-alkyl, C₁-C₆-alkoxy, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{az},
C(O)NR^{a}R^{b}, (SO₂)NR^{a}R^{b}, and radicals of the formula Y-Cy, wherein
Y is a single bond, oxygen, sulfur or C₁-C₆-alkandiyl, wherein one carbon might be replaced with oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, or C₂-C₆-alkynyl, wherein the carbon atoms in these groups may carry any combination of 1, 2 or 3 radicals R^{aw};
R^{c} have one of the meanings given for R^{a} and R^{b} or are independently of each other selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino.
R^{a2}, R^{a3}, R^{a4}, R^{a5}, R^{a6}, R^{a7}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{b6}, R^{b7}, R^{bc} and R^{bz} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a} and R^{24b} have independently one of the meanings given for R^{b1} or two radicals R²¹ bound to the same carbon atom may together with this carbon atom form a carbonyl group;
provided that if A is —C(R^{6a})(R^{6b})—, at least one of the radicals R¹, R^{3a}, R^{3b}, R^{3c}, R^{3d}, R^{4a}, R^{4b}, R^{6a} or R^{6b} is different from hydrogen.

30. The method of claim 29 wherein, A is oxygen, NR⁷, sulfur, S(O) or S(O)₂.

31. The method of claim 29, wherein A is C(O).

32. The method of claim 29, wherein A is CH₂.

33. The method of claim 29, wherein phenyl carries 1, 2, 3, 4 or 5 radicals R^{z1}, which are independently of each other selected from the group consisting of halogen, C₁-C₆-alkyl, C₁-C₆-alkoxy, C₁-C₆-haloalkoxy, C₁-C₆-alkylthio and C₁-C₆-haloalkylthio.

34. The method of claim 29, wherein R^{2a} and R^{2b} are selected from the group consisting of hydrogen, C₁-C₆-alkyl, formyl, CN, C₁-C₆-alkylcarbonyl, C₁-C₆-haloalkylcarbonyl, C₁-C₆-alkoxycarbonyl, C₁-C₄-alkoxy-C₁-C₄-alkoxycarbonyl or C₁-C₆-alkylthiocarbonyl.

35. The method of claim 34, wherein R^{2a} or R^{2b} are hydrogen.

36. The method of claim 29, wherein R^{2a} and R^{2b} are selected from the group consisting of oxazolyl, thiazolyl and imidazolyl.

37. The method of claim 29, wherein each of the radicals R^{3a}, R^{3b}, R^{3c} and R^{3d} is hydrogen.

38. The method of claim 29, wherein R^{4a} is selected from the group consisting of hydrogen, halogen, C₁-C₆-alkyl, C₂-C₆-alkenyl, C₂-C₆-alkynyl, phenyl, 5 or 6 membered hetaryl, 5 or 6 membered hetarylmethyl and benzyl wherein the four last mentioned radicals may be unsubstituted or may carry any combination of 1, 2, 3, 4 or 5 radicals R^{b4}.

39. The method of claim 29, wherein R^{4a} and R^{4b} are independently selected from the group consisting of hydrogen, halogen, C₁-C₆-alkyl, C₂-C₆-alkenyl, C₂-C₆-alkynyl, phenyl, 5 or 6 membered hetaryl, 5 or 6 membered hetarylmethyl and benzyl wherein the four last mentioned radicals may be unsubstituted or may carry any combination of 1, 2, 3, 4 or 5 radicals R^{b4}.

40. The method of claim 29, wherein R^{4b} is hydrogen or C₁-C₆-alkyl.

41. The method of claim 29, wherein B is a single bond.

42. The method of claim 29, wherein B is CH₂.

43. The method of claim 29, wherein R¹ is different from hydrogen.

44. The method of claim 29, wherein X is S.

45. The method of claim 29, wherein X is O.

46. The method of claim 29, wherein X is NR⁵.

47. A method of protecting growing plants from attack or infestation by insects or nematodes, which comprises applying to the plants, or to the soil or water in which they are growing, at least one compound of formula (I.a) or (I.b) and/or an agriculturally acceptable salt thereof: 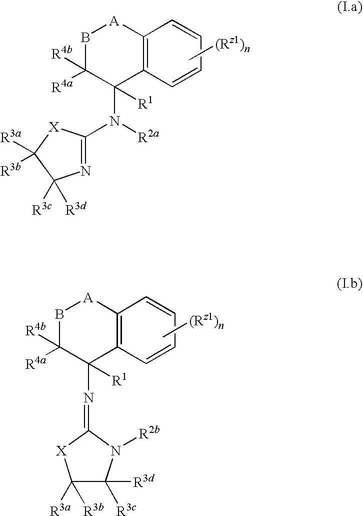
wherein
n is 0, 1, 2, 3 or 4.
X is sulfur, oxygen or a radical NR⁵;
A is —C(R^{6a})(R^{6b})—, oxygen, NR⁷, sulfur, S(O) or S(O)₂;
B is a chemical bond or CH₂;
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl or benzyl, phenoxycarbonyl, 5 or 6 membered hetaryl and 5 or 6 membered hetarylmethyl each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl, a 5 or 6 membered heterocyclic ring and benzoyl each of the seven last mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members, and wherein the 5 or 6 membered heterocyclic ring contains 1, 2 or 3 heteroatoms selected from the group consisting of oxygen, sulfur and nitrogen as ring members; or R¹ together with R^{2a} may be C₃-C₅-alkandiyl which may carry 1, 2, 3, 4 or 5 radicals R²¹ and which may be interrupted with 1 or 2 heteroatoms selected from the group consisting of oxygen, sulfur or nitrogen;
R¹ together with R^{2b} may also be a bridging carbonyl group C(O);
R^{3a}, R^{3b}, R^{3c}, R^{3d} are each independently selected from the group consisting of hydrogen, halogen, cyano, nitro, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, each of the last three mentioned radicals may be unsubstituted or may carry any combination of 1, 2, 3, 4 or 5 radicals R^{b3};
R^{4a}, R^{4b} are independently from each other selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
phenyl, benzyl, 5 or 6 membered hetaryl and 5 or 6 membered hetarylmethyl, each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O, ═NR^{c} or ═CR^{d}R^{e};
R^{2a} together with R^{4a} may form a bridging bivalent radical selected from the group consisting of C(O)—C(R^{24a})(R^{24b}), C(S)—C(R^{24a})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, S(O)—NH.
R⁵ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{6a}, R^{6b} are independently of each other selected from the group consisting of hydrogen, halogen, cyano, nitro, hydroxy, mercapto, amino, C₁-C₆-alkyl, C₁-C₆-haloalkyl, C₁-C₆-alkoxy, C₁-C₆-alkylamino, di-(C₁-C₆-alkyl)amino, wherein the carbon atoms in the last 5 mentioned radicals may be unsubstituted or may carry any combination of 1, 2 or 3 radicals R^{a6},
C₃-C₆-cycloalkyl, phenyl andbenzyl, each of the last three mentioned radicals may be unsubstituted or may carry any combination of 1, 2, 3, 4 or 5 radicals R^{b6}; or
R^{6a} together with R^{6b} may also be ═O, ═NR^{c} or ═CR^{d}R^{e};
R⁷ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a7},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b7}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{z1} are independently of each other selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{az},
C(O)NR^{a}R^{b}, (SO₂)NR^{a}R^{b}, and radicals of the formula Y-Cy, wherein
Y is a single bond, oxygen, sulfur or C₁-C₆-alkandiyl, wherein one carbon might be replaced with oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, or C₂-C₆-alkynyl, wherein the carbon atoms in these groups may carry any combination of 1, 2 or 3 radicals R^{aw};
R^{c} have one of the meanings given for R^{a} and R^{b} or are independently of each other selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino.
R^{a2}, R^{a3}, R^{a4}, R^{a5}, R^{a5}, R^{a7}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{b6}, R^{b7}, R^{bc} and R^{bz} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a} and R^{24b} have independently one of the meanings given for R^{b1} or two radicals R²¹ bound to the same carbon atom may together with this carbon atom form a carbonyl group;
provided that if A is —C(R^{6a})(R^{6b})—, at least one of the radicals R¹, R^{3a}, R^{3b}, R^{3c}, R^{3d}, R^{4a}, R^{4b}, R^{6a} or R^{6b} is different from hydrogen.

48. The method of claim 47, wherein the compound of formula (I.a) or (I.b) and/or the salt thereof or a composition comprising them is applied in an amount of from 5 g/ha to 2000 g/ha, calculated as the compound of formula (I.a) or (I.b).

49. A method of protection of seed comprising contacting the seeds with a compound of formula (I.a) or (I.b) and/or an agriculturally acceptable salt thereof: 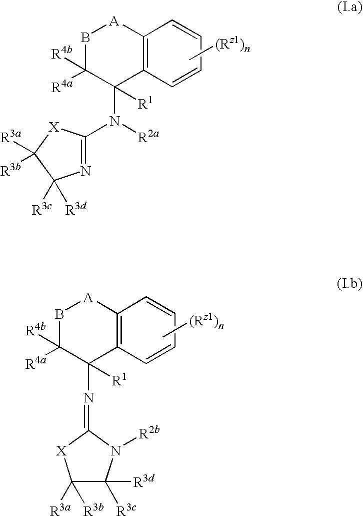
wherein
n is 0, 1, 2, 3 or 4.
X is sulfur, oxygen or a radical NR⁵;
A is —C(R^{6a})(R^{6b})—, oxygen, NR⁷, sulfur, S(O) or S(O)₂;
B is a chemical bond or CH₂;
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl or benzyl, phenoxycarbonyl, 5 or 6 membered hetaryl and 5 or 6 membered hetarylmethyl each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl, a 5 or 6 membered heterocyclic ring and benzoyl each of the seven last mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members, and wherein the 5 or 6 membered heterocyclic ring contains 1, 2 or 3 heteroatoms selected from the group consisting of oxygen, sulfur and nitrogen as ring members; or
R¹ together with R^{2a} may be C₃-C₅-alkandiyl which may carry 1, 2, 3, 4 or 5 radicals R²¹ and which may be interrupted with 1 or 2 heteroatoms selected from the group consisting of oxygen, sulfur or nitrogen;
R¹ together with R^{2b} may also be a bridging carbonyl group C(O);
R^{3a}, R^{3b}, R^{3c}, R^{3d} are each independently selected from the group consisting of hydrogen, halogen, cyano, nitro, mercapto, amino, C₁-C₆-haloalkyl, C₁-C₆-alkyl, C₁-C₆-alkylamino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, each of the last three mentioned radicals may be unsubstituted or may carry any combination of 1, 2, 3, 4 or 5 radicals R^{b3};
R^{4a}, R^{4b} are independently from each other selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
phenyl, benzyl, 5 or 6 membered hetaryl and 5 or 6 membered hetarylmethyl, each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O, ═NR^{C} or ═CR^{d}R^{e};
R^{2a} together with R^{4a} may form a bridging bivalent radical selected from the group consisting of C(O)—C(R^{24a})(R^{24b}), C(S)—C(R^{24a})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, S(O)—NH.
R⁵ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{6a}, R^{6b} are independently of each other selected from the group consisting of hydrogen, halogen, cyano, nitro, hydroxy, mercapto, amino, C₁-C₆-alkyl, C₁-C₆-haloalkyl, C₁-C₆-alkoxy, C₁-C₆-alkylamino, di-(C₁-C₆-alkyl)amino, wherein the carbon atoms in the last 5 mentioned radicals may be unsubstituted or may carry any combination of 1, 2 or 3 radicals R^{a6},
C₃-C₆-cycloalkyl, phenyl andbenzyl, each of the last three mentioned radicals may be unsubstituted or may carry any combination of 1, 2, 3, 4 or 5 radicals R^{b6}; or
R^{6a} together with R^{6b} may also be ═O, ═NR^{c} or ═CR^{d}R^{e};
R⁷ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a7},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b7}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{z1} are independently of each other selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{az},
C(O)NR^{a}R^{b}, (SO₂)NR^{a}R^{b}, and radicals of the formula Y-Cy, wherein
Y is a single bond, oxygen, sulfur or C₁-C₆-alkandiyl, wherein one carbon might be replaced with oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, or C₂-C₆-alkynyl, wherein the carbon atoms in these groups may carry any combination of 1, 2 or 3 radicals R^{aw};
R^{c} have one of the meanings given for R^{a} and R^{b} or are independently of each other selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino.
R^{a2}, R^{a3}, R^{a4}, R^{a5}, R^{a6}, R^{a7}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{b6}, R^{b7}, R^{bc} and R^{bz} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a} and R^{24b} have independently one of the meanings given for R^{b1} or two radicals R²¹ bound to the same carbon atom may together with this carbon atom form a carbonyl group;
provided that if A is —C(R^{6a})(R^{6b})—, at least one of the radicals R¹, R^{3a}, R^{3b}, R^{3c}, R^{3d}, R^{4a}, R^{4b}, R^{6a} or R^{6b} is different from hydrogen in a pesticidally effective amount.

50. The method of claim 49 wherein the compound of formula (I.a) or (I.b) and/or an agriculturally acceptable salt thereof or a composition comprising the compound of formula (I.a) or (I.b) is applied in an amount of from 0.1 g to 10 kg per 100 kg of seeds.

51. A seed comprising a compound of formula (I.a) or (I.b) and/or an agriculturally acceptable salt thereof 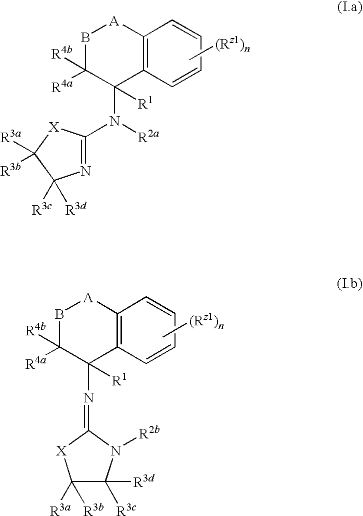
wherein
n is 0, 1, 2, 3 or 4.
X is sulfur, oxygen or a radical NR⁵;
A is —C(R^{6a})(R^{6b})—, oxygen, NR⁷, sulfur, S(O) or S(O)₂;
B is a chemical bond or CH₂;
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl or benzyl, phenoxycarbonyl, 5 or 6 membered hetaryl and 5 or 6 membered hetarylmethyl each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl, a 5 or 6 membered heterocyclic ring and benzoyl each of the seven last mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members, and wherein the 5 or 6 membered heterocyclic ring contains 1, 2 or 3 heteroatoms selected from the group consisting of oxygen, sulfur and nitrogen as ring members; or
R¹ together with R^{2a} may be C₃-C₅-alkandiyl which may carry 1, 2, 3, 4 or 5 radicals R²¹ and which may be interrupted with 1 or 2 heteroatoms selected from the group consisting of oxygen, sulfur or nitrogen;
R¹ together with R^{2b} may also be a bridging carbonyl group C(O);
R^{3a}, R^{3b}, R^{3c}, R^{3d} are each independently selected from the group consisting of hydrogen, halogen, cyano, nitro, mercapto, amino, C₁-C₆-haloalkyl, C₁-C₆-alkyl, C₁-C₆-alkylamino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, each of the last three mentioned radicals may be unsubstituted or may carry any combination of 1, 2, 3, 4 or 5 radicals R^{b3};
R^{4a}, R^{4b} are independently from each other selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
phenyl, benzyl, 5 or 6 membered hetaryl and 5 or 6 membered hetarylmethyl, each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O, ═NR^{c} or ═CR^{d}R^{e};
R^{2a} together with R^{4a} may form a bridging bivalent radical selected from the group consisting of C(O)—C(R^{24a})(R^{24b}), C(S)—C(R^{24a})(R^{24b}), CH₂—(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, S(O)—NH.
R⁵ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{6a}, R^{6b} are independently of each other selected from the group consisting of hydrogen, halogen, cyano, nitro, hydroxy, mercapto, amino, C₁-C₆-alkyl, C₁-C₆-alkoxy, di-(C₁-C₆-alkyl)amino, wherein the carbon atoms in the last 5 mentioned radicals may be unsubstituted or may carry any combination of 1, 2 or 3 radicals R^{a6},
C₃-C₆-cycloalkyl, phenyl andbenzyl, each of the last three mentioned radicals may be unsubstituted or may carry any combination of 1, 2, 3, 4 or 5 radicals R^{b6}; or
R^{6a} together with R^{6b} may also be ═O, ═NR^{c} or ═CR^{d}R^{e};
R⁷ is selected from the group consisting of hydrogen, formyl, CN, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a7},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b7}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
are independently of each other selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{az},
C(O)NR^{a}R^{b}, (SO₂)NR^{a}R^{b}, and radicals of the formula Y-Cy, wherein
Y is a single bond, oxygen, sulfur or C₁-C₆-alkandiyl, wherein one carbon might be replaced with oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, or C₂-C₆-alkynyl, wherein the carbon atoms in these groups may carry any combination of 1, 2 or 3 radicals R^{aw};
R^{c} have one of the meanings given for R^{a} and R^{b} or are independently of each other selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino.
R^{a2}, R^{a3}, R^{a4}, R^{a5}, R^{a6}, R^{a7}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{b6}, R^{b7}, R^{bc} and R^{bz} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyDamino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a} and R^{24b} have independently one of the meanings given for R^{b1} or two radicals R²¹ bound to the same carbon atom may together with this carbon atom form a carbonyl group;
provided that if A is —C(R^{6a})(R^{6b}), at least one of the radicals R¹, R^{3a}, R^{3b}, R^{3c}, R^{3d}, R^{4a}, R^{4b}, R^{6a} or R^{6b} is different from hydrogen.
in an amount of from 0.1 g to 10 kg per 100 kg of seeds, calculated as the compound of formula (I.a) or (I.b).

52. A method for treating, controlling, preventing or protecting animals against infestation or infection by endoparasites which comprises administering or applying to the animals a parasiticidally effective amount of at least one compound of formula (I.a) or (I.b) and/or an veterinarily acceptable salt thereof 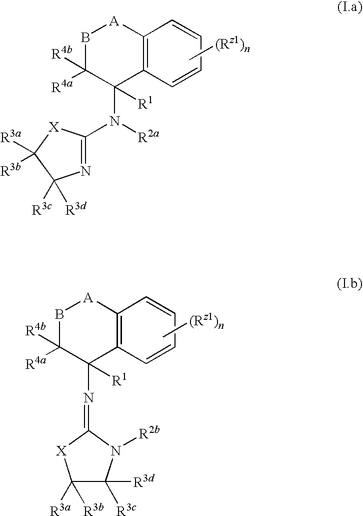
wherein
n is 0, 1, 2, 3 or 4.
X is sulfur, oxygen or a radical NR⁵;
A is —C(R^{6a})(R^{6b})—, oxygen, NR⁷, sulfur, S(O) or S(O)₂;
B is a chemical bond or CH₂;
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl or benzyl, phenoxycarbonyl, 5 or 6 membered hetaryl and 5 or 6 membered hetarylmethyl each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl, a 5 or 6 membered heterocyclic ring and benzoyl each of the seven last mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members, and wherein the 5 or 6 membered heterocyclic ring contains 1, 2 or 3 heteroatoms selected from the group consisting of oxygen, sulfur and nitrogen as ring members; or
R¹ together with R^{2a} may be C₃-C₅-alkandiyl which may carry 1, 2, 3, 4 or 5 radicals R²¹ and which may be interrupted with 1 or 2 heteroatoms selected from the group consisting of oxygen, sulfur or nitrogen;
R¹ together with R^{2b} may also be a bridging carbonyl group C(O);
R^{3a}, R^{3b}, R^{3c}, R^{3d} are each independently selected from the group consisting of hydrogen, halogen, cyano, nitro, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, each of the last three mentioned radicals may be unsubstituted or may carry any combination of 1, 2, 3, 4 or 5 radicals R^{b3};
R^{4a}, R^{4b} are independently from each other selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
phenyl, benzyl, 5 or 6 membered hetaryl and 5 or 6 membered hetarylmethyl, each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O, ═NR^{c} or ═CR^{d}R^{e};
R^{2a} together with R^{4a} may form a bridging bivalent radical selected from the group consisting of C(O)—C(R^{24a})(R^{24b}), C(S)—C(R^{24a})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, S(O)—NH.
R⁵ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{6a}, R^{6b} are independently of each other selected from the group consisting of hydrogen, halogen, cyano, nitro, hydroxy, mercapto, amino, C₁-C₆-alkyl, C₁-C₆-haloalkyl, C₁-C₆-alkoxy, C₁-C₆-alkylamino, di-(C₁-C₆-alkyl)amino, wherein the carbon atoms in the last 5 mentioned radicals may be unsubstituted or may carry any combination of 1, 2 or 3 radicals R^{a6},
C₃-C₆-cycloalkyl, phenyl andbenzyl, each of the last three mentioned radicals may be unsubstituted or may carry any combination of 1, 2, 3, 4 or 5 radicals R^{b6}; or
R^{6a} together with R^{6b} may also be ═O, ═NR^{c} or ═CR^{d}R^{e};
R⁷ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a7},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b7}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{z1} are independently of each other selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-Cₒ-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{az},
C(O)NR^{a}R^{b}, (SO₂)NR^{a}R^{b}, and radicals of the formula Y-Cy, wherein
Y is a single bond, oxygen, sulfur or C₁-C₆-alkandiyl, wherein one carbon might be replaced with oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, or C₂-C₆-alkynyl, wherein the carbon atoms in these groups may carry any combination of 1, 2 or 3 radicals R^{aw};
R^{c} have one of the meanings given for R^{a} and R^{b} or are independently of each other selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino.
R^{a2}, R^{a3}, R^{a4}, R^{a5}, R^{a6}, R^{a7}, R^{aw} and R^{az} are indendently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{b6}, R^{b7}, R^{bc} and R^{bz} are independently selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a} and R^{24b} have independently one of the meanings given for R^{b1} or two radicals R²¹ bound to the same carbon atom may together with this carbon atom form a carbonyl group;
provided that if A , is —C(R^{6a})(R^{6b})—, at least one of the radicals R¹, R^{3a}, R^{3b}, R^{3c}, R^{3d}, R^{4a}, R^{4b}, R^{6a} or R^{6b} is different from hydrogen.

53. Azoline compounds of the general formula (I.a) or (I.b), 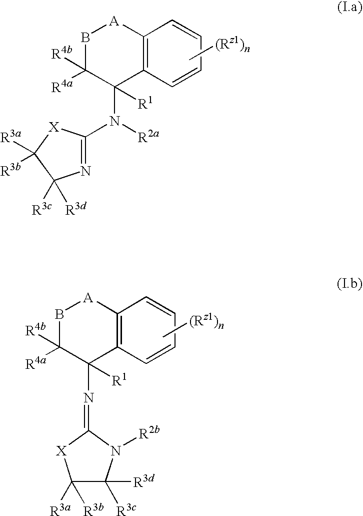
wherein
n is 0, 1, 2, 3 or 4.
X is sulfur, oxygen or a radical NR⁵;
A , is —C(R^{6a})(R^{6b})—, oxygen, NR⁷, sulfur, S(O) or S(O)₂;
B is a chemical bond or CH₂;
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl or benzyl, phenoxycarbonyl, 5 or 6 membered hetaryl and 5 or 6 membered hetarylmethyl each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl, a 5 or 6 membered heterocyclic ring and benzoyl each of the seven last mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members, and wherein the 5 or 6 membered heterocyclic ring contains 1, 2 or 3 heteroatoms selected from the group consisting of oxygen, sulfur and nitrogen as ring members; or
R¹ together with R^{2a} may be C₃-C₅-alkandiyl which may carry 1, 2, 3, 4 or 5 radicals R²¹ and which may be interrupted with 1 or 2 heteroatoms selected from the group consisting of oxygen, sulfur or nitrogen;
R¹ together with R^{2b} may also be a bridging carbonyl group C(O);
R^{3a}, R^{3b}, R^{3c}, R^{3d} are each independently selected from the group consisting of hydrogen, halogen, cyano, nitro, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, each of the last three mentioned radicals may be unsubstituted or may carry any combination of 1, 2, 3, 4 or 5 radicals R^{b3};
R^{4a}, R^{4b} are independently from each other selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
phenyl, benzyl, 5 or 6 membered hetaryl and 5 or 6 membered hetarylmethyl, each of the four last mentioned radicals may be unsubstituted or may carry any combination of 1, 2, 3, 4 or 5 radicals R^{m}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O, ═NR^{c} or ═CR^{d}R^{e};
R^{2a} together with R^{4a} may form a bridging bivalent radical selected from the group consisting of C(O)—C(R^{24a})(R^{24b}), C(S)—C(R^{24a})(R^{24b}), CH₂—(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, S(O)—NH.
R⁵ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{6a}, R^{6b} are independently of each other selected from the group consisting of hydrogen, halogen, cyano, nitro, hydroxy, mercapto, amino, C₁-C₆-alkyl, C₁-C₆-haloalkyl, C₁-C₆-alkoxy, C₁-C₆-alkylamino, di-(C₁-C₆-alkyl)amino, wherein the carbon atoms in the last 5 mentioned radicals may be unsubstituted or may carry any combination of 1, 2 or 3 radicals R^{a6},
C₃-C₆-cycloalkyl, phenyl andbenzyl, each of the last three mentioned radicals may be unsubstituted or may carry any combination of 1, 2, 3, 4 or 5 radicals R^{b6}; or
R^{6a} together with R^{6b} may also be ═O, ═NR^{c} or ═CR^{d}R^{e};
R⁷ is selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a7},
C(O)NR^{a}R^{b}, C(S)NR^{a}R^{b}, (SO₂)NR^{a}R^{b},
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b7}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
R^{z1} are independently of each other selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{az},
C(O)NR^{a}R^{b}, (SO₂)NR^{a}R^{b}, and radicals of the formula Y-Cy, wherein
Y is a single bond, oxygen, sulfur or C₁-C₆-alkandiyl, wherein one carbon might be replaced with oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, or C₂-C₆-alkynyl, wherein the carbon atoms in these groups may carry any combination of 1, 2 or 3 radicals R^{aw};
R^{c} have one of the meanings given for R^{a} and R^{b} or are independently of each other selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino.
R^{a2}, R^{a3}, R^{a4}, R^{a5}, R^{a6}, R^{a7}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{b6}, R^{b7}, R^{bc} and R^{bz} independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a} and R^{24b} have independently one of the meanings given for R^{b1} or two radicals R²¹ bound to the same carbon atom may together with this carbon atom form a carbonyl group;
provided that if A is —C(R^{6a})(R^{6b})—, at least one of the radicals R¹, R^{3a}, R^{3b}, R^{3c}, R^{3d}, R^{4a}, R^{4b}, R^{6a} or R^{6b} is different from hydrogen
provided that,
if X is oxygen, then at least one of the radicals R¹ or R^{2a} or R^{2b} is different from hydrogen, and/or A is different from —C(R^{6a})(R^{6b})—, oxygen, sulfur, N(CH₃), or if X is oxygen and B is a chemical bond, then A is different from —C(R^{6a})(R^{6b})—,
and provided that, if X is nitrogen or sulfur, then at least one of the radicals R¹ or R^{2a} or R^{2b} is different from hydrogen, and/or A is different from —C(R^{6a})(R^{6b})—,
and the salts thereof.

54. Compositions comprising the compound of claim 53 and a carrier material.
